(19) United States
(12) Patent Application Publication
(10) Pub. No.: US 2026/0297452 A1
(43) Pub. Date: Oct. 1, 2026

(54) FLUORINE-CONTAINING ETHER COMPOUND, LUBRICANT FOR MAGNETIC RECORDING MEDIUM, AND MAGNETIC RECORDING MEDIUM

(71) Applicant: Resonac Corporation, Tokyo (JP)

(72) Inventors: Yutaka TANJI, Tokyo (JP); Kohei SAGA, Tokyo (JP); Daisuke YAGYU, Tokyo (JP); Takuya UNO, Tokyo (JP)

(73) Assignee: Resonac Corporation, Tokyo (JP)

(21) Appl. No.: 19/480,217

(22) PCT Filed: Nov. 21, 2024
(86) PCT No.: PCT/JP2024/041281

§ 371 Date: Oct. 30, 2025

(30) Foreign Application Priority Data
Nov. 28, 2023 (JP) 2023-200893

(51) Int. Cl. C10M 147/04; C08G 65/34; C10N 40/18; G11B 5/725

(52) U.S. Cl. CPC C10M 147/04; C08G 65/34; G11B 5/7257; C10M 2213/06; C10N 2040/18

(57) ABSTRACT

A fluorine-containing ether compound represented by the following formula:

R¹—CH₂—R²[—CH₂—R³—CH₂—R²]ₓ—CH₂—R⁴

where x is 0 to 2, R² is a perfluoropolyether chain, R³ is a divalent linking group having 1 to 4 polar groups, R¹ and R⁴ are terminal groups having 1 to 4 polar groups and having 1 to 50 carbon atoms, and at least one of R¹ and R⁴ is a formula (2)

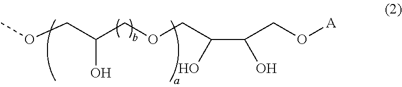

where a is 0 to 2, and b is 1 to 6, and A is an organic group optionally including at least one of a polar group, a halogeno group, and an ether oxygen atom and having 1 to 30 carbon atoms or a hydrogen atom.

### TECHNICAL FIELD

The present invention relates to a fluorine-containing ether compound, a lubricant for a magnetic recording medium, and a magnetic recording medium.

Priority is claimed on Japanese Patent Application No. 2023-200893, filed Nov. 28, 2023, the content of which is incorporated herein by reference.

### BACKGROUND ART

In order to improve the recording density of a magnetic recording and reproducing device, development of a magnetic recording medium suitable for a high recording density is underway.

Conventionally, there have been magnetic recording media having a recording layer formed on a substrate and a protective layer made of carbon or the like formed on the recording layer. The protective layer protects information recorded in the recording layer and enhances the sliding properties with respect to magnetic heads. However, durability of a magnetic recording medium cannot be sufficiently obtained only by providing the protective layer on the recording layer. Therefore, generally, a lubricating layer is formed by applying a lubricant onto the surface of the protective layer.

As the lubricant that is used at the time of forming the lubricating layer of the magnetic recording medium, for example, a lubricant containing a compound having a polar group, such as a hydroxyl group or an amino group, at a terminal of a fluorine-based polymer having a repeating structure including —CF₂— has been proposed.

For example, Patent Document 1 and Patent Document 2 disclose fluorine-containing ether compounds having glycerin structures (—O—CH₂—CH(OH)—CH₂—O—) and terminal groups that are each an organic group having a polar group bonding in this order to both terminals of a perfluoropolyether chain through methylene groups (—CH₂—).

Patent Document 3 discloses a fluorine-containing ether compound having linking groups with a structure in which an ether bond (—O—), a methylene group (—CH₂—), and a methylene group (—CH(OH)—), in which one hydrogen atom has been substituted with a hydroxyl group, are combined together disposed between a perfluoropolyether chain and both terminal groups, respectively.

Patent Document 4 discloses a fluorine-containing ether compound having a skeleton in which two perfluoropolyether chains have bonded to both ends of a glycerin structure (—O—CH₂—CH(OH)—CH₂—O—) through divalent linking groups to which a methylene group (—CH₂—) has bonded, in which terminal groups having a polar group have bonded to both terminals thereof through methylene groups.

### CITATION LIST

### Patent Document

Patent Document 1: PCT International Publication No. WO 2021/090940 (A)

Patent Document 2: Japanese Patent No. 6804893 (B)

Patent Document 3: PCT International Publication No. WO 2019/054148 (A)

Patent Document 4: U.S. Pat. No. 10,540,997 (B)

### SUMMARY OF INVENTION

### Technical Problem

Toward an increase in the capacities of magnetic recording and reproducing devices, development of magnetic recording media suitable for a high recording density is underway. Recently, in order to improve the recording densities of magnetic recording media, there has been a demand to further shorten the distance between a magnetic head and a magnetic layer in a magnetic recording medium and reduce magnetic spacing (flying height). Therefore, there has been a demand to further thin the thickness of a lubricating layer in a magnetic recording medium.

However, generally, when the thickness of the lubricating layer is made to be thin, the chemical substance resistance of the magnetic recording medium tends to deteriorate. In addition, when the thickness of the lubricating layer is made to be thin, there is a case where it becomes impossible to maintain a film thickness thick enough to satisfy the function of the lubricating layer due to spinoff. Spinoff refers to a phenomenon in which a lubricant is scattered or evaporated due to a centrifugal force associated with the rotation of the magnetic recording medium and heat generated. From these facts, there is a demand for a lubricating layer having excellent chemical substance resistance and being capable of curbing spinoff even when the thickness of the lubricating layer is made to be thin.

The present invention has been made in consideration of the above-described circumstance, and an object of the present invention is to provide a fluorine-containing ether compound that enables the formation of a lubricating layer having excellent chemical substance resistance and being capable of curbing spinoff and that can be suitably used as a material of lubricants for magnetic recording media.

In addition, another object of the present invention is to provide a lubricant for a magnetic recording medium that contains the fluorine-containing ether compound of the present invention and enables the formation of a lubricating layer in which the chemical substance resistance is favorable and spinoff can be curbed.

In addition, still another object of the present invention is to provide a magnetic recording medium having a lubricating layer containing the fluorine-containing ether compound of the present invention, in which the chemical substance resistance is favorable and spinoff is curbed.

### Solution to Problem

The present invention includes the following aspects.

[1] A fluorine-containing ether compound that is represented by a following formula (1):

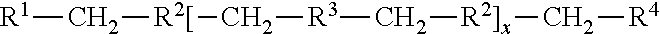

(in the formula (1), x represents an integer of 0 to 2. R² is a perfluoropolyether chain. In a case where x is 1 or 2, (x+1) R²'s may be partially or fully identical to each other or may be different from each other. R³ is a divalent linking group having 1 to 4 polar groups. In a case where x is 2, two R³'s may be identical to each other or may be different from each other. R¹ and R⁴ are each independently a terminal group having 1 to 4 polar groups and having 1 to 50 carbon atoms. At least one of Ri and R⁴ is a terminal group represented by a following formula (2).)

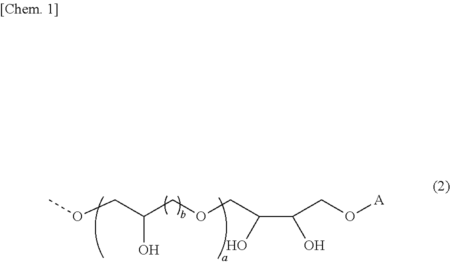

(in the formula (2), a represents an integer of 0 to 2, and b represents an integer of 1 to 6. A is an organic group or a hydrogen atom, the organic group having 1 to 30 carbon atoms and optionally including at least one of a polar group, a halogeno group, and an ether oxygen atom. Here, the total number of the polar groups included in the formula (2) is 2 to 4. In a case where a is 2, two b's may be identical to each other or may be different from each other.)

[2] The fluorine-containing ether compound according to [1], in which the terminal group represented by the formula (2) is any of following formulae (2-1) to (2-6).

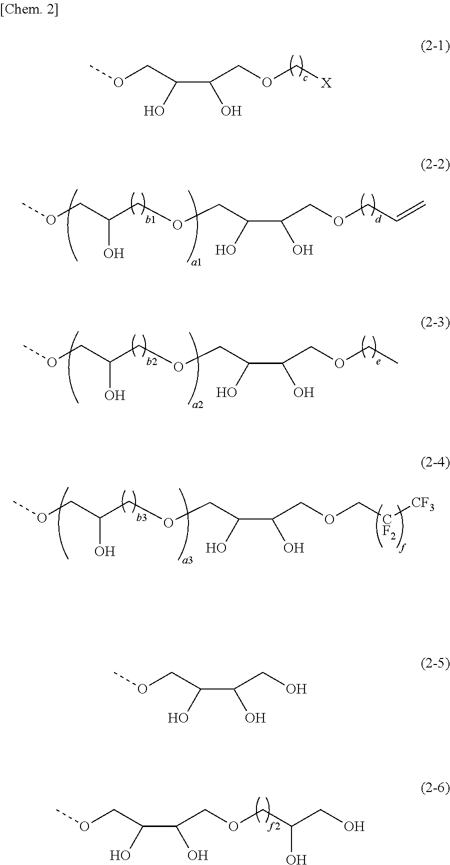

(in the formula (2-1), c represents an integer of 1 to 6. X represents any of a hydroxyl group, an acetamide group, a carboxamide group, and a cyano group.)
(in the formula (2-2), a1 represents 0 or 1. b1 represents an integer of 1 to 6. d represents an integer of 1 to 4.)
(in the formula (2-3), a2 represents 0 or 1. b2 represents an integer of 1 to 6. e represents an integer of 0 to 7.)
(in the formula (2-4), a3 represents 0 or 1. b3 represents an integer of 1 to 6. f represents an integer of 0 to 6.)
(in the formula (2-6), f2 represents an integer of 1 to 6.)

[3] The fluorine-containing ether compound according to [1] or [2], in which R¹ and R⁴ in the formula (1) are each independently the terminal group represented by the formula (2).

[4] The fluorine-containing ether compound according to [3], in which R¹ and R⁴ in the formula (1) are identical to each other.

[5] The fluorine-containing ether compound according to [1] or [2], in which only one of R¹ and R⁴ in the formula (1) is the terminal group represented by the formula (2), and other is a terminal group represented by formula (3).

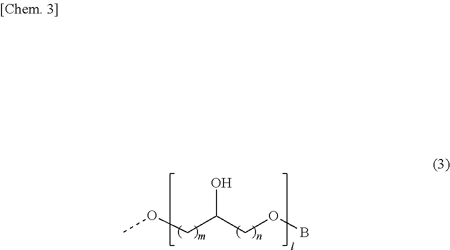

(in the formula (3), 1 represents an integer of 1 to 3. l m's each independently represent an integer of 1 to 6. l n's each independently represent an integer of 1 to 6. In one repeating unit, at least one of m and n is 1. B represents an alkyl group, an organic group or a hydrogen atom, the alkyl group optionally having only one polar group, the organic group including a carbon-carbon unsaturated bond and optionally having only one polar group.)

[6] The fluorine-containing ether compound according to [1], [2], or [5], in which only one of R¹ and R⁴ in the formula (1) is the terminal group represented by the formula (2), and other is a terminal group represented by any of following formulae (3-1) to (3-3).

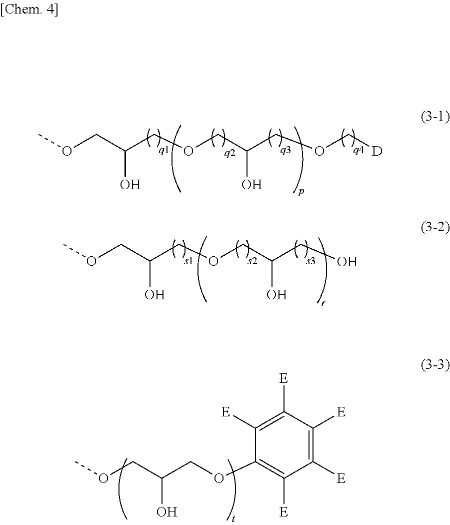

(in the formula (3-1), p represents 0 or 1. q1, q2, q3, and q4 each independently represent an integer of 1 to 6. In a case where p is 0, a total value of q1 and q4 is 2 to 10. In a case where p is 1, a total value of q1, q2, q3, and q4 is 4 to 10. In a case where p is 1, at least one of q2 and q3 is 1. D represents a polar group, a vinyl group, an ethynyl group, or an aryl group, the aryl group optionally having a substituent. Here, in a case where D is the aryl group optionally having a substituent, a number of polar groups included in D is 0 or 1.)
(in the formula (3-2), r represents 0 or 1. s1, s2, and s3 each independently represent an integer of 1 to 6. In a case where r is 1, a total value of s1, s2, and s3 is 3 to 8. In a case where r is 1, at least one of s2 and s3 is 1.)
(in the formula (3-3), t represents 1 or 2. Five E's each independently represent a polar group, an alkoxy group, a halogeno group, or a hydrogen atom, the alkoxy group having 1 to 8 carbon atoms. Here, in a case where a polar group is included in the five E's, a total number of the polar groups in the five E's is one.)

[7] The fluorine-containing ether compound according to [1] or [2], in which only one of R¹ and R⁴ in the formula (1) is the terminal group represented by the formula (2), and other is a terminal group represented by following formulae (3-4) or (3-5).

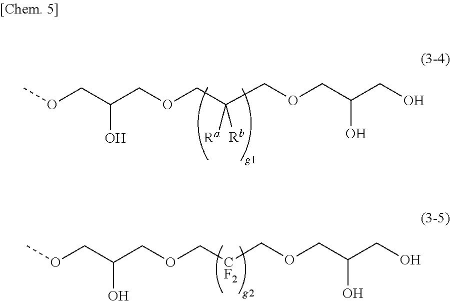

(in the formula (3-4), g1 represents an integer of 1 to 6. g1 R^{a}'s and g1 R^{b}'s are each independently a hydrogen atom or a methyl group.)
(in the formula (3-5), g2 represents an integer of 1 to 6.)

[8] The fluorine-containing ether compound according to any one of [1] to [7], in which x in the formula (1) is 1 or 2, and x R³'s are each independently a divalent linking group having one to three hydroxyl groups, having oxygen atoms at both end portions bonding to adjacent methylene groups, and having 3 to 50 carbon atoms.

[9] The fluorine-containing ether compound according to any one of [1] to [8], in which x in the formula (1) is 1 or 2, and x R³'s are each independently any one selected from linking groups represented by following formulae (4-1) to (4-6).

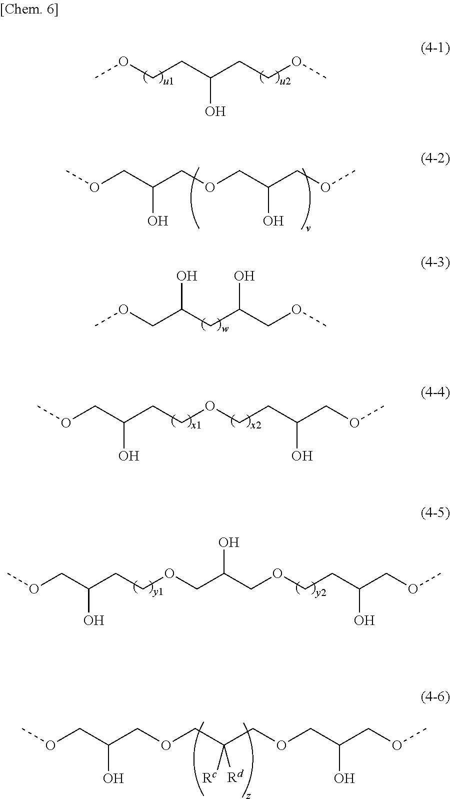

(in the formula (4-1), u1 represents an integer of 0 to 6, and u2 represents an integer of 0 to 6. Here, at least one of u1 and u2 is 0. An oxygen atom at a left terminal of the formula (4-1) bonds to a methylene group on an R¹ side in the formula (1), and an oxygen atom at a right terminal bonds to a methylene group on an R⁴ side in the formula (1).)
(in the formula (4-2), v represents an integer of 1 or 2. An oxygen atom at a left terminal of the formula (4-2) bonds to the methylene group on the Ri side in the formula (1), and an oxygen atom at a right terminal bonds to the methylene group on the R⁴ side in the formula (1).)
(in the formula (4-3), w represents an integer of 0 to 6. An oxygen atom at a left terminal of the formula (4-3) bonds to the methylene group on the R¹ side in the formula (1), and an oxygen atom at a right terminal bonds to the methylene group on the R⁴ side in the formula (1).)
(in the formula (4-4), x1 represents an integer of 0 to 5, and x2 represents an integer of 0 to 5. Here, at least one of x1 and x2 is an integer of 1 to 5. An oxygen atom at a left terminal of the formula (4-4) bonds to the methylene group on the R¹ side in the formula (1), and an oxygen atom at a right terminal bonds to the methylene group on the R⁴ side in the formula (1).)
(in the formula (4-5), y1 represents an integer of 1 to 5, and y2 represents an integer of 1 to 5. An oxygen atom at a left terminal of the formula (4-5) bonds to the methylene group on the R¹ side in the formula (1), and an oxygen atom at a right terminal bonds to the methylene group on the R⁴ side in the formula (1).)
(in the formula (4-6), z represents an integer of 1 to 6. z R^{c}'s and z R^{d}'s are each independently a hydrogen atom, a fluorine atom, or a methyl group. An oxygen atom at a left terminal of the formula (4-6) bonds to the methylene group on the R¹ side in the formula (1), and an oxygen atom at a right terminal bonds to the methylene group on the R⁴ side in the formula (1).)

[10] The fluorine-containing ether compound according to any one of [1] to [9], in which (x+1) R²'s in the formula (1) are each independently a perfluoropolyether chain represented by a following formula (5).

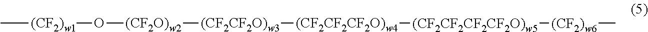

(in the formula (5), w2, w3, w4, and w5 indicate average degrees of polymerization and each independently represent 0 to 20. Here, w2, w3, w4, and w5 are not all 0 at the same time. w1 and w6 are average values representing the numbers of CF₂'s and each independently represent 1 to 3. An array order of (CF₂O), (CF₂CF₂O), (CF₂CF₂CF₂O), and (CF₂CF₂CF₂CF₂O), which are repeating units in the formula (5), is not particularly limited.)

[11] The fluorine-containing ether compound according to any one of [1] to [10], in which (x+1) R²'s in the formula (1) are each independently any one selected from perfluoropolyether chains represented by following formulae (5-1) to (5-4).

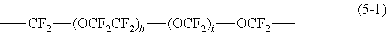

(in the formula (5-1), h and i indicate average degrees of polymerization, h represents 1 to 20, and i represents 0 to 20.)

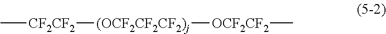

(in the formula (5-2), j indicates an average degree of polymerization and represents 1 to 15.)

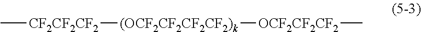

(in the formula (5-3), k indicates an average degree of polymerization and represents 1 to 10.)

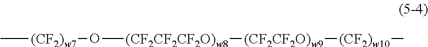

(in the formula (5-4), w8 and w9 indicate average degrees of polymerization and each independently represent 1 to 20. w7 and w10 are average values representing the numbers of CF₂'s and each independently represent 1 or 2.)

The fluorine-containing ether compound according to [1], in which the fluorine-containing ether compound represented by the formula (1) is any one of following formulae (AA), (AB), (AI), (AJ), (AN) to (AQ), (AS), (AW), (BA) to (BM), (CA), and (DA).

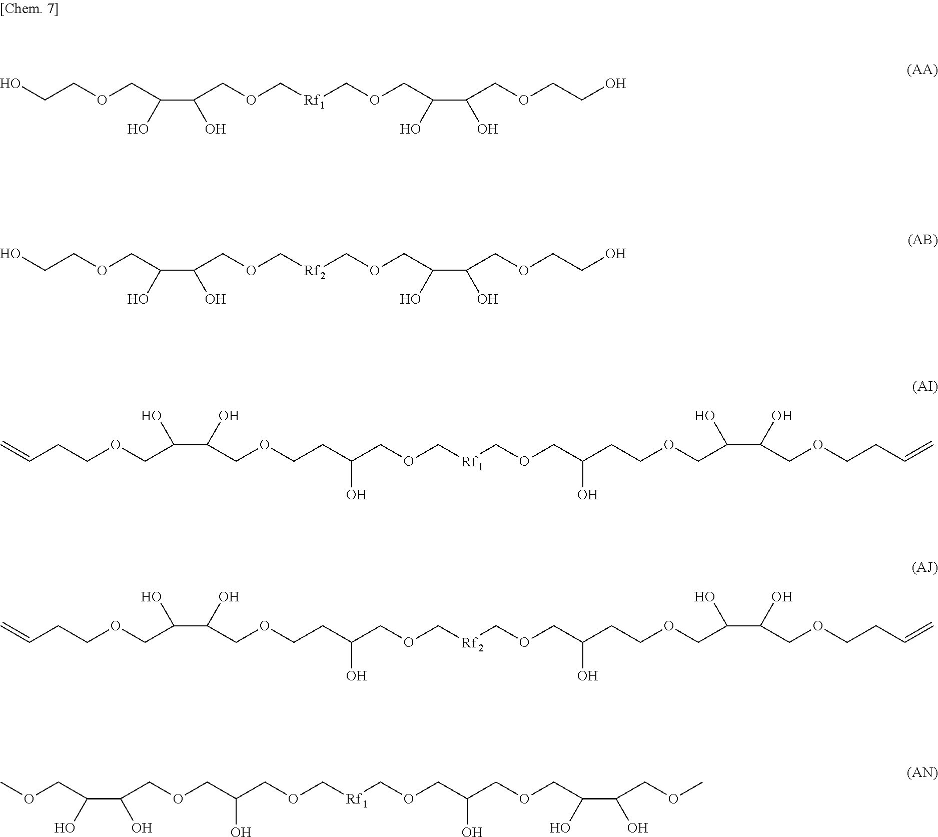

(in Rf₁ in the formula (AA), h and i indicate average degrees of polymerization, h represents 1 to 20, and i represents 0 to 20.)
(in Rf₂ in the formula (AB), j indicates an average degree of polymerization and represents 1 to 15.)
(in Rf₁ in the formula (AI), h and i indicate average degrees of polymerization, h represents 1 to 20, and i represents 0 to 20.)
(in Rf₂ in the formula (AJ), j indicates an average degree of polymerization and represents 1 to 15.)
(in Rf₁ in the formula (AN), h and i indicate average degrees of polymerization, h represents 1 to 20, and i represents 0 to 20.)

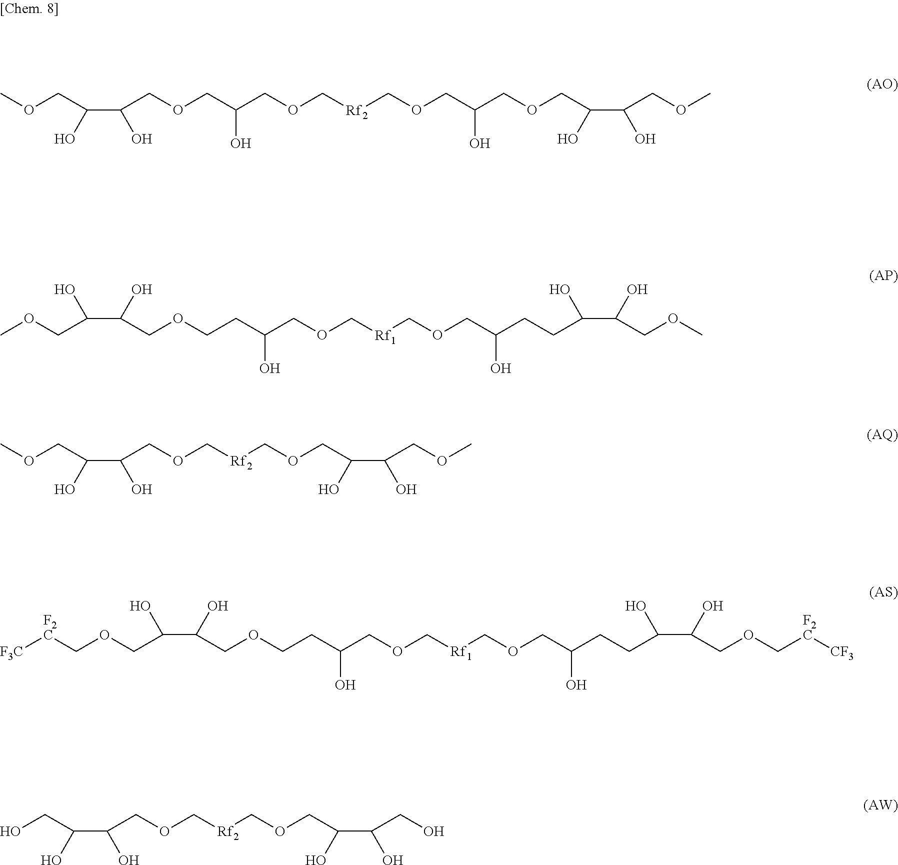

(in Rf₂ in the formula (AO), j indicates average degrees of polymerization and represents 1 to 15.)
(in Rf₁ in the formula (AP), h and i indicate average degrees of polymerization, h represents 1 to 20, and i represents 0 to 20.)
(in Rf₂ in the formula (AQ), j indicates an average degree of polymerization and represents 1 to 15.)
(in Rf₂ in the formula (AS), j indicates an average degree of polymerization and represents 1 to 15.)
(in Rf₂ in the formula (AW), j indicates an average degree of polymerization and represents 1 to 15.)

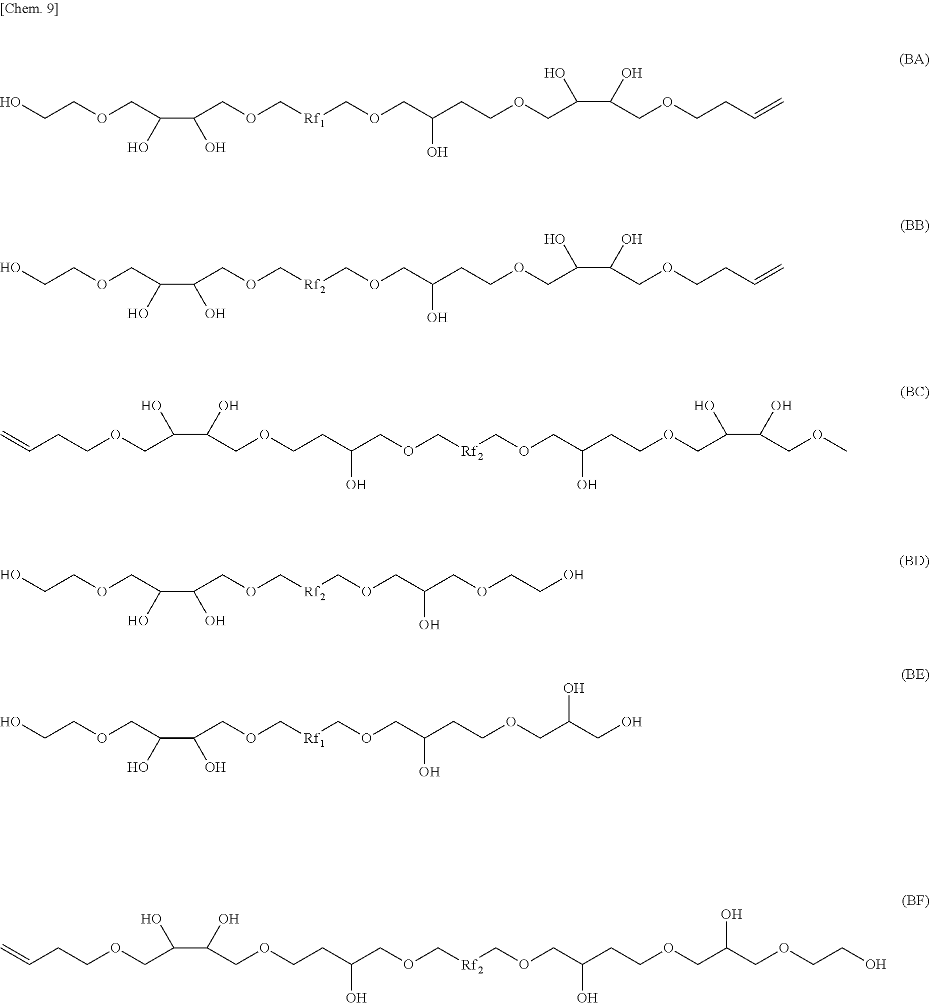

(in Rf₁ in the formula (BA), h and i indicate average degrees of polymerization, h represents 1 to 20, and i represents 0 to 20.)
(in Rf₂ in the formula (BB), j indicates an average degrees of polymerization and represents 1 to 15.)
(in Rf₂ in the formula (BC), j indicates an average degree of polymerization and represents 1 to 15.)
(in Rf₂ in the formula (BD), j indicates an average degree of polymerization and represents 1 to 15.)
(in Rf₁ in the formula (BE), h and i indicate average degrees of polymerization, h represents 1 to 20, and i represents 0 to 20.)
(in Rf₂ in the formula (BF), j indicates an average degree of polymerization and represents 1 to 15.)

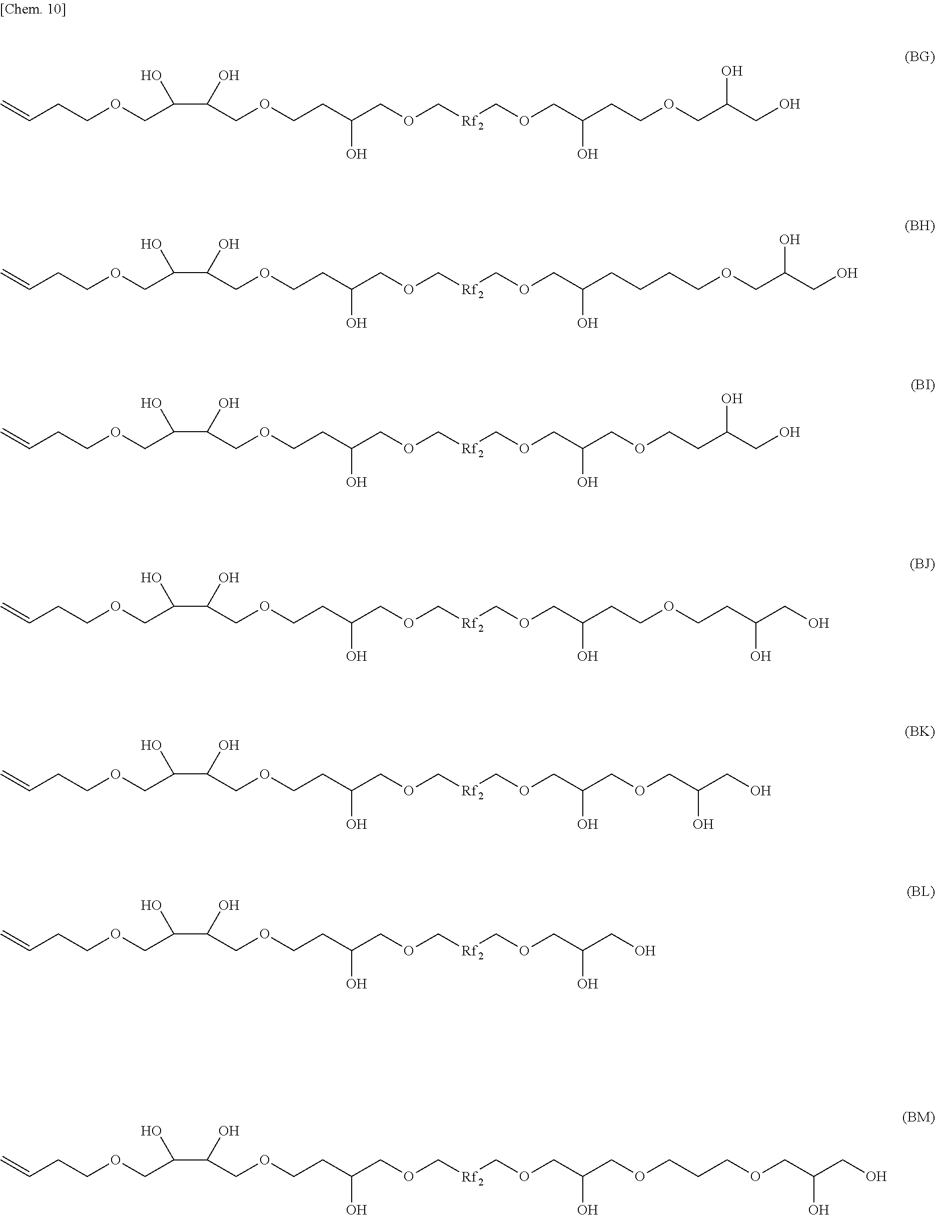

(in Rf₂ in the formula (BG), j indicates an average degree of polymerization and represents I to 15.1)
(in Rf₂ in the formula (BH), j indicates an average degree of polymerization and represents 1 to 15.)
(in Rf₂ in the formula (BI), j indicates an average degree of polymerization and represents 1 to 15.)
(in Rf₂ in the formula (BJ), j indicates an average degree of polymerization and represents 1 to 15.)
(in Rf₂ in the formula (BK), j indicates an average degree of polymerization and represents 1 to 15.)
(in Rf₂ in the formula (BL), j indicates an average degree of polymerization and represents 1 to 15.)
(in Rf₂ in the formula (BM), j indicates an average degree of polymerization and represents 1 to 15.)

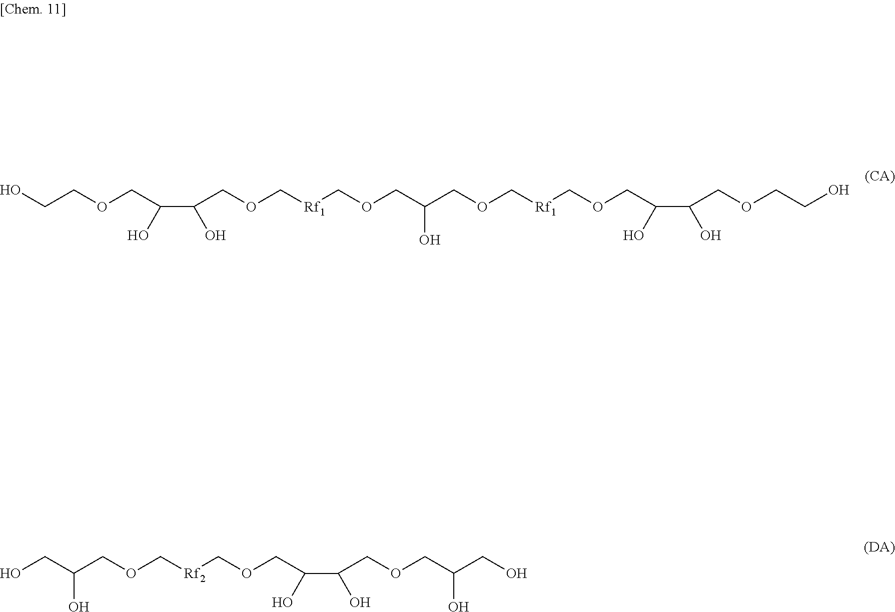

(in two Rf₁'s in the formula (CA), h and i indicate average degrees of polymerization, h represents 1 to 20, and i represents 0 to 20. h's and i's in the two Rf₁'s may be identical to each other or may be different from each other.)
(in Rf₂ in the formula (DA), j indicates an average degree of polymerization and represents 1 to 15.)

[13] The fluorine-containing ether compound according to any one of [1] to [12], in which a number-average molecular weight is within a range of 500 to 10000.

[14] A lubricant for a magnetic recording medium containing the fluorine-containing ether compound according to any one of [1] to [13].

[15] A magnetic recording medium having at least a magnetic layer, a protective layer, and a lubricating layer provided in order on a substrate,
in which the lubricating layer contains the fluorine-containing ether compound according to any one of [1] to [13].

[16] The magnetic recording medium according to [16], in which the lubricating layer has an average film thickness of 0.5 nm to 2.0 nm.

### Advantageous Effects of Invention

The fluorine-containing ether compound of the present invention is a compound represented by the formula (1) and is thus suitable as a material of lubricants for magnetic recording media.

The lubricant for magnetic recording medium of the present invention contains the fluorine-containing ether compound of the present invention and thus enables the formation of a lubricating layer having favorable chemical substance resistance and being capable of curbing spinoff.

The magnetic recording medium of the present invention has a lubricating layer containing the fluorine-containing ether compound of the present invention. Therefore, the magnetic recording medium of the present invention has favorable chemical substance resistance, is capable of curbing spinoff, and is excellent in terms of reliability and durability. In addition, the magnetic recording medium of the present invention has a lubricating layer having favorable chemical substance resistance and being capable of curbing spinoff and is thus capable of thinning the thickness of the lubricating layer, capable of contributing to reduction in magnetic spacing, and capable of further decreasing the flying height of a magnetic head.

### BRIEF DESCRIPTION OF DRAWINGS

FIG. 1 A schematic cross-sectional view showing one embodiment of a magnetic recording medium of the present invention.

### DESCRIPTION OF EMBODIMENTS

In order to achieve the above-described objects, the present inventors intensively studied as described below.

Conventionally, as a material of a lubricant for a magnetic recording medium (hereinafter abbreviated as “lubricant” in some cases) that is applied on the surface of a protective layer, a fluorine-containing ether compound having a terminal group including a polar group, such as a hydroxyl group, is preferably used.

However, in the case of forming a lubricating layer of thin thickness on a protective layer using the conventional fluorine-containing ether compound having a terminal group including a polar group, it has been difficult to realize a lubricating layer having favorable chemical substance resistance and being capable of curbing spinoff as described below.

That is, the polar group in the fluorine-containing ether compound bonds to an active point on the protective layer to improve the adhesion of the lubricating layer to the protective layer. When the adhesion of the lubricating layer to the protective layer is not sufficient, the coating state of the lubricating layer on the protective layer becomes insufficient, a chemical contamination substance is likely to be incorporated into the protective layer, and sufficient chemical substance resistance cannot be obtained.

In addition, the polar group in the fluorine-containing ether compound does not only bond to an active point on the protective layer and get involved in the interaction with the protective layer but also gets involved in interactions in the molecule and between the molecules. When the number of the polar groups that get involved in the interaction between the molecules of the fluorine-containing ether compound is not sufficient, the fluorine-containing ether compound in the lubricating layer is easily scattered due to the rotation of magnetic recording media, and spinoff is likely to occur.

Therefore, the fluorine-containing ether compound needs to sufficiently include polar groups that get involved in the interaction with the protective layer and polar groups that get involved in the interaction between the molecules so as to enable the formation of a lubricating layer in which chemical substance resistance is favorable and spinoff is curbed. However, when the number of the polar groups in the fluorine-containing ether compound increases, the polar groups are likely to attract chemical contamination substances, which makes it impossible to obtain sufficient chemical substance resistance.

Therefore, the present inventors repeated intensive studies with attention paid to the strengths of polar groups that are included in a fluorine-containing ether compound and the interaction between the polar groups. As a result, the present inventors considered that in a fluorine-containing ether compound including a specific number of polar groups, it is preferable to substitute at least some of the polar groups with hydroxyl groups of a terminal group including an erythritol structure (—O—CH₂—CH(OH)—CH(OH)—CH₂—O—) disposed at one or both terminals of a skeleton including a perfluoropolyether chain. In this case, as described below, it is possible to ensure the number of hydroxyl groups capable of interacting with a protective layer and the number of hydroxyl groups capable of getting involved in interactions between the molecules while an increase in surface free energy attributed to the hydroxyl groups that are included in the erythritol structure is curbed.

That is, in an internal 1,2-diol structure (—CH₂—CH(OH)—CH(OH)—CH₂—) that is included in the erythritol structure (—O—CH₂—CH(OH)—CH(OH)—CH₂—O—), carbon atoms to which the hydroxyl groups have bonded bond to each other. Therefore, the distance between the hydroxyl groups is close, and steric repulsion and electrostatic repulsion between the hydroxyl groups are likely to occur. In addition, in the erythritol structure, since a carbon atom to which the hydroxyl group has bonded and a carbon atom to which the hydroxyl group has bonded bond to each other, free rotation is likely to be curbed. From these facts, the two hydroxyl groups that are included in the erythritol structure are in reversed conformation with respect to a carbon chain in the erythritol structure. As a result, dipole moments that are generated from the two hydroxyl groups included in the erythritol structure cancel each other, and the surface free energy of the entire molecule of the fluorine-containing ether compound decreases.

In addition, the distance between the active points on the protective layer is sufficiently large compared with the distance between the hydroxyl groups that are included in the erythritol structure. Furthermore, as described above, the two hydroxyl groups that are included in the erythritol structure are in reversed conformation with respect to the carbon chain in the erythritol structure. Therefore, the two hydroxyl groups that are included in the erythritol structure are oriented toward to the protective layer at the same time. Therefore, only one hydroxyl group between the two hydroxyl groups is capable of interacting with the active point on the protective layer, and the other hydroxyl group is capable of getting involved in the interaction between the molecules of the fluorine-containing ether compound.

Conventionally, regarding the structure of a fluorine-containing ether compound that is used in lubricants, studies have been made to dispose as many polar groups as possible in the compound to easily interact with active points on a protective layer to improve the adhesion to the protective layer. Therefore, there has been a tendency to avoid a structure in which the orientations of adjacent hydroxyl groups are in reversed conformation on the protective layer due to the bond between carbon atoms to which the hydroxyl groups have bonded as in the erythritol structure.

However, the present inventors, conversely, ensured the number of polar groups capable of getting involved in the interaction between the molecules of a fluorine-containing ether compound by substituting some of the polar groups in the fluorine-containing ether compound with hydroxyl groups in an erythritol structure. On top of that, the present inventors considered that it is preferable to adjust the number of polar groups capable of interacting with active points on a protective layer as necessary to an extent that an increase in surface free energy attributed to the excessively large number of the polar groups can be curbed.

Furthermore, regarding a fluorine-containing ether compound having a terminal group including an erythritol structure disposed in at least one terminal of a skeleton including a perfluoropolyether chain, the present inventors repeated studies to improve the chemical substance resistance of a lubricating layer containing this fluorine-containing ether compound and curb spinoff.

As a result, the present inventors found that a fluorine-containing ether compound represented by a formula (1) in which terminal groups disposed at the terminals of a skeleton including a perfluoropolyether chain (and a linking group disposed between the perfluoropolyether chains in the case of having a plurality of perfluoropolyether chains) each have a predetermined number of polar groups and a terminal group represented by a formula (2) including an erythritol structure (—O—CH₂—CH(OH)—CH(OH)—CH₂—O—) is present in at least one terminal of the skeleton including a perfluoropolyether chain is preferable.

In the fluorine-containing ether compound represented by the formula (1), due to a synergistic effect of <1> to <3> to be described below, a hydroxyl group that gets involved in neither an interaction with a protective layer nor an interaction in the molecule is generated, and a polar group capable of getting involved in an interaction between the molecules is easily formed. Therefore, in a lubricating layer containing the fluorine-containing ether compound represented by the formula (1), it is assumed to be that scattering of the fluorine-containing ether compound associated with the rotation of magnetic recording media is less likely to occur and spinoff can be curbed.

In addition, in the fluorine-containing ether compound represented by the formula (1), due to the synergistic effect of <1> to <3> to be described below, an interaction between the polar groups in the molecule is curbed, and a hydroxyl group capable of getting involved in the interaction between the molecules is easily formed. Therefore, the number of polar groups capable of getting involved in the interaction with the protective layer and the interaction between the molecules can be sufficiently ensured even when the number of the polar groups that are included in the compound is small. Therefore, it is possible to curb an increase in surface free energy attributed to an increase in the number of the polar groups that are included in the fluorine-containing ether compound. As a result, in the lubricating layer containing the fluorine-containing ether compound represented by the formula (1), it is assumed to be that it is possible to prevent the incorporation of a chemical contamination substance caused by the polar groups that are included in the fluorine-containing ether compound attracting the chemical contamination substance, an interaction with the chemical contamination substance is small, and the chemical substance resistance is excellent.

<1> Since the two hydroxyl groups in the erythritol structure are in reversed conformation with respect to the carbon chain in the erythritol structure, only one of the two hydroxyl groups is capable of interacting with an active point on a protective layer. Therefore, the number of hydroxyl groups capable of interacting the protective layer and the number of hydroxyl groups that do not get involved in the interaction with the protective layer but are capable of getting involved in an interaction between the molecules can be easily ensured, and it is possible to curb an increase in surface free energy attributed to an increase in the number of polar groups in the compound compared with a compound not including the erythritol structure.

<2> The erythritol structure is curbed in motion due to its bulkiness and thus brings appropriate rigidity to the fluorine-containing ether compound molecule. Due to this fact, the erythritol structure curbs the interaction between the polar groups in the molecule, and it becomes easy to get the polar groups in the compound involved in the interaction between the molecules of the fluorine-containing ether compound.

<3> In the terminal group represented by the formula (2), oxygen atoms are disposed at both ends in the erythritol structure. Since these oxygen atoms easily freely rotate, the internal 1,2-diol structure that is included in the terminal group represented by the formula (2) is independently mobile. Therefore, the internal 1,2-diol structure in the erythritol structure in the compound is less likely to be inhibited from getting involved in the intermolecular interaction.

In contrast, when the mobility of the fluorine-containing ether compound is too high, it becomes easy for the polar groups that are included in the fluorine-containing ether compound to form an intramolecular interaction. Therefore, the polar groups are less likely to get involved in the intermolecular interaction.

In addition, in a case where all of a plurality of polar groups that are included in the fluorine-containing ether compound are disposed to be sufficiently distant and apart from adjacent polar groups, all of the polar groups are likely to get involved in the interaction with the protective layer, and the interaction between the molecules of the fluorine-containing ether compound by the polar groups cannot be sufficiently obtained.

Furthermore, the present inventors confirmed that the use of a lubricant containing the fluorine-containing ether compound represented by the formula (1) enables the formation of a lubricating layer having favorable chemical substance resistance and being capable of curbing spinoff even when the thickness is made to be thin and came to an idea of the present invention.

Hereinafter, a fluorine-containing ether compound, a lubricant for a magnetic recording medium, and a magnetic recording medium of the present invention will be described in detail. The present invention is not limited only to embodiments to be described below. “Polar group” in the present specification does not include halogeno groups (—F, —Cl, —Br, and the like) or an ether bond (—O—).

### Fluorine-Containing Ether Compound

A fluorine-containing ether compound of the present embodiment is represented by the following formula (1).

R¹—CH₂—R²[—CH₂—R³—CH₂—R²]ₓ—CH₂—R⁴ (1)
(in the formula (1), x represents an integer of 0 to 2. R² is a perfluoropolyether chain. In a case where x is 1 or 2, (x+1) R²'s may be partially or fully identical to each other or may be different from each other. R³ is a divalent linking group having 1 to 4 polar groups. In a case where x is 2, two R³'s may be identical to each other or may be different from each other. R¹ and R⁴ are each independently a terminal group having 1 to 4 polar groups and having 1 to 50 carbon atoms. At least one of R¹ and R⁴ is a terminal group represented by the following formula (2).)

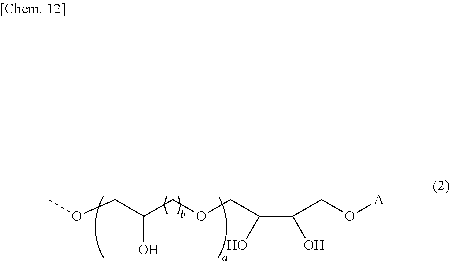

(in the formula (2), a represents an integer of 0 to 2, and b represents an integer of 1 to 6. A is an organic group optionally including at least one of a polar group, a halogeno group, and an ether oxygen atom and having 1 to 30 carbon atoms or a hydrogen atom. Here, the total number of the polar groups included in the formula (2) is 2 to 4. In a case where a is 2, two b's may be identical to each other or may be different from each other.)

In the fluorine-containing ether compound of the present embodiment, as shown in the formula (1), in a case where x is 0, the terminal group indicated by Ri is bonded to one end of the perfluoropolyether chain indicated by R² (hereinafter referred to as the PFPE chain in some cases) through a methylene group and the terminal group indicated by R⁴ is bonded to the other end through a methylene group.

In addition, in a case where x is 1 or 2, the fluorine-containing ether compound has a skeleton in which the divalent linking groups indicated by R³ and two or three perfluoropolyether chains indicated by R² are alternately linked to one another through methylene groups. The terminal group indicated by Ri is bonded to one end of the skeleton through a methylene group and the terminal group indicated by R⁴ is bonded to the other end of the skeleton through a methylene group.

In the fluorine-containing ether compound represented by the formula (1), the number (x+1) of the PFPE chains indicated by R² is 1 to 3. In a case where x is 1 or 2, in the fluorine-containing ether compound represented by the formula (1), R³, which is a divalent linking group having 1 to 4 polar groups, is disposed between the adjacent R²'s.

The fluorine-containing ether compound represented by the formula (1) curbs the molecule becoming too large compared with a compound having 4 or more PFPE chains. From this fact, the fluorine-containing ether compound represented by the formula (1) easily spreads on the protective layer and enables the formation of a lubricating layer having a thin and uniform film thickness compared with the compound having 4 or more PFPE chains. In order to form a more uniform lubricating layer, x is preferably 0 or 1 and more preferably 0.

Terminal Groups Indicated by R¹ and R⁴

R¹ and R⁴ are each independently a terminal group having 1 to 4 polar groups and having 1 to 50 carbon atoms. At least one of R¹ and R⁴ is a terminal group represented by the formula (2).

In a case where one of R¹ and R⁴ is a terminal group that does not correspond to the formula (2), the number of polar groups that are included in the terminal group that does not correspond to the formula (2) between R¹ and R⁴ is one or more. In addition, the number of polar groups that are included in the terminal group represented by the formula (2) is two or more. Therefore, in the case of forming a lubricating layer on a protective layer using a lubricant containing the fluorine-containing ether compound, a suitable interaction is generated between the lubricating layer and the protective layer. The numbers of the polar groups that are included in R¹ and R⁴ are each preferably two or more. When the numbers of the polar groups that are included in R¹ and R⁴ are each two or more, it becomes easy to ensure the number of polar groups capable of getting involved in the interaction with the protective layer and the interaction between the molecules. Therefore, the fluorine-containing ether compound has superior adhesion to the protective layer and enables the formation of a lubricating layer capable of curbing spinoff.

The numbers of the polar groups that are included in R¹ and R⁴ are each four or less. Therefore, in the lubricating layer containing the fluorine-containing ether compound, it is possible to curb the polarity of the fluorine-containing ether compound being too high, the fluorine-containing ether compound aggregating to be lumps, and the lubricating layer losing smoothness. In addition, when the numbers of the polar groups that are included in R¹ and R⁴ are each four or less, it is possible to prevent an excessive increase in the surface free energy of the fluorine-containing ether compound and the incorporation of a chemical contamination substance into magnetic recording media, and it becomes possible to form a lubricating layer having high chemical substance resistance from the fluorine-containing ether compound. The numbers of the polar groups that are included in R¹ and R⁴ are each preferably three or less.

The total number of the polar groups that are included in R¹ and R⁴ in the formula (1) is preferably three or more and six or less and more preferably four or more and six or less. Since the total number of the polar groups is three or more, the interaction between the polar groups in R¹ and R⁴ and the protective layer can be effectively obtained. As a result, it becomes possible to form a lubricating layer having high adhesion to the protective layer from the fluorine-containing ether compound, and a lubricating layer having excellent chemical substance resistance and spinoff resistance can be obtained. In addition, when the total number of the polar groups is six or less, it is possible to prevent an excessive increase in the surface free energy of the fluorine-containing ether compound and the incorporation of a chemical contamination substance into a lubricating layer. Therefore, it is possible to form a lubricating layer having superior chemical substance resistance.

The polar groups that are included in each of R¹ and R⁴ may be partially or fully identical to each other or may be different from each other. In addition, the number of the polar groups in R¹ and the number of the polar groups in R⁴ may be identical to each other or may be different from each other. The number of the polar groups in R¹ and the number of the polar groups in R⁴ are preferably identical to each other since the coating state of the fluorine-containing ether compound on the protective layer becomes more uniform, and it becomes possible to form a lubricating layer having more favorable adhesion.

The polar group that is included in R¹ and R⁴ is preferably at least one polar group selected from the group consisting of a hydroxyl group (—OH), a carboxy group (—COOH), a formyl group (—(C═O)H), a carbonyl group (—(C═O)R⁷; R⁷ is an organic group), a sulfo group (—SO₃H), a cyano group (—CN), a group having an amide bond (—NR⁸COR⁹ or —CONR¹⁰R¹¹; R⁸, R⁹, R¹⁰, and R¹¹ are each independently a hydrogen atom or an organic group), and an amino group (—NR¹²R¹³; R¹² and R¹³ are each independently a hydrogen atom or an organic group). The group having an amide bond includes both a group that bonds at a carbon atom configuring the amide bond (for example, a carboxamide group (—C(═O)NH₂)) and a group that bonds at a nitrogen atom configuring the amide bond (for example, an acetamido group (—NHC(═O)CH₃)) as shown in the above-described formula. In the group having an amide bond, R⁸ and R⁹ may bond to each other to form a ring, and R¹⁰ and R¹¹ may bond to each other to form a ring. R⁸, R⁹, R¹⁰, and R¹¹ in the group having an amide bond are each independently preferably selected from the group consisting of a hydrogen atom, a methyl group, an ethyl group, a propyl group, and a butyl group.

R¹ and R⁴ each independently preferably include at least one polar group selected from the group consisting of a hydroxyl group, a cyano group, and a group having an amide bond. This is because the hydroxyl group, the cyano group, and the group having an amide bond are chemically stable and a lubricating layer containing the fluorine-containing ether compound having these polar groups does not alter for a long period of time. In addition, this is because the hydroxyl group, the cyano group, and the group having an amide bond have an acidity that is not too high and are less likely to corrode substrates.

In a case where one of R¹ and R⁴ is the terminal group that does not correspond to the formula (2), the terminal group that does not correspond to the formula (2) preferably includes at least one hydroxyl group. This is because the coating state of the fluorine-containing ether compound on the protective layer becomes more uniform.

Even in a case where only one of R¹ and R⁴ is the terminal group represented by the formula (2) or a case where both R¹ and R⁴ are the terminal groups represented by the formula (2), the polar groups in R¹ and the polar groups in R⁴ are all preferably hydroxyl groups. This is because when the polar groups in R¹ and R⁴ are all hydroxyl groups, the coating state of the fluorine-containing ether compound on the protective layer becomes more uniform.

In a case where one of R¹ and R⁴ is the terminal group that does not correspond to the formula (2), the number of carbon atoms that are included in the terminal group that does not correspond to the formula (2) between R¹ and R⁴ is one or more. In addition, the terminal group represented by the formula (2) includes at least four or more carbon atoms that the erythritol structure (—O—CH₂—CH(OH)—CH(OH)—CH₂—O—) has. In the fluorine-containing ether compound represented by the formula (1), since the numbers of carbon atoms in the terminal groups represented by R¹ and R⁴ are one or more, it is possible to maintain the surface free energy of the terminal groups low. Therefore, it is possible to prevent a chemical contamination substance from being attracted to the lubricating layer, and a lubricating layer having favorable chemical substance resistance can be formed. In a case where one of R¹ and R⁴ is the terminal group that does not correspond to the formula (2), the number of carbon atoms that are included in the terminal group that does not correspond to the formula (2) between R¹ and R⁴ is preferably three or more and more preferably four or more.

In a case where one of R¹ and R⁴ is the terminal group that does not correspond to the formula (2), the number of carbon atoms that are included in the terminal group that does not correspond to the formula (2) between R¹ and R⁴ is 50 or less. In addition, the terminal group represented by the formula (2) includes 16 or less carbon atoms in a glycerin structure (—O—CH₂—CH(OH)—CH₂—O—) and/or a structure obtained by extending carbon chains in the glycerin structure with (b-1) methylene groups, four carbon atoms in the erythritol structure, and 30 or less carbon atoms in A and has a total of 50 or less carbon atoms. Since the numbers of carbon atoms in the terminal groups represented by R¹ and R⁴ are each 50 or less, the terminal groups become flexible structures, and the adhesion between the lubricating layer containing the fluorine-containing ether compound and the protective layer becomes favorable. As a result, it becomes possible to form a lubricating layer capable of curbing spinoff. The numbers of carbon atoms in the terminal groups represented by R¹ and R⁴ are each preferably 20 or less and more preferably 15 or less.

### Terminal Group Represented by the Formula (2)

At least one of R¹ and R⁴ is the terminal group represented by the formula (2). The terminal group represented by the formula (2) has an oxygen atom (ether oxygen atom) that is bonded to a methylene group (—CH₂—) bonding to R². The oxygen atom that is bonded to the methylene group bonding to R² forms ether bonds (—O—) with atoms that are bonded to both sides thereof. This ether bond imparts appropriate flexibility to the fluorine-containing ether compound represented by the formula (1) and increases the affinity between the polar group in the terminal group represented by the formula (2) and the protective layer. This makes the fluorine-containing ether compound represented by the formula (1) enable the formation of a lubricating layer having excellent adhesion to the protective layer.

The terminal group represented by the formula (2) has an erythritol structure (—O—CH₂—CH(OH)—CH(OH)—CH₂—O—). Therefore, the lubricating layer containing the fluorine-containing ether compound represented by the formula (1) has favorable chemical substance resistance and is capable of curbing spinoff.

A in the formula (2) is an organic group optionally including at least one of a polar group, a halogeno group, and an ether oxygen atom and having 1 to 30 carbon atoms or a hydrogen atom.

The terminal group represented by the formula (2) has the organic group having 1 to 30 carbon atoms or the hydrogen atom that is represented by A. In a case where A is the organic group having 1 to 30 carbon atoms, since the number of carbon atoms in A is 1 or more, the terminal group becomes appropriately rigid, and the intramolecular interaction between the polar groups that are included in the fluorine-containing ether compound is curbed. In addition, since the number of the carbon atoms in A is 30 or less, it is possible to curb the interaction between the polar group and the protective layer being inhibited due to the fact that the terminal group represented by the formula (2) becomes too bulky and the motion of the polar group that is included in the fluorine-containing ether compound is hindered. In a case where A is the organic group having 1 to 30 carbon atoms, the number of the carbon atoms in A is preferably 1 to 15 and more preferably 2 to 10.

In the formula (2), in a case where A is an organic group having 1 to 30 carbon atoms and a is 0, A may include one or two polar groups. In addition, in a case where A is an organic group having 1 to 30 carbon atoms and a is 1, A may include one polar group. In addition, in a case where A is an organic group having 1 to 30 carbon atoms and a is 2, A does not include a polar group. In a case where A includes one or two polar groups, the adhesion of the lubricating layer containing the fluorine-containing ether compound represented by the formula (1) to the protective layer becomes more favorable, and a sufficient coating state can be obtained even when the thickness is made to be thin. In addition, in a case where A includes polar groups, the number of the polar groups is preferably one. In this case, it is possible to curb chemical substances being attracted from the environment due to the surface free energy of the fluorine-containing ether compound represented by the formula (1) becoming too high.

In a case where A in the formula (2) is an organic group including a polar group and having 1 to 30 carbon atoms, the polar group is preferably a polar group selected from the group consisting of a hydroxyl group, a cyano group, and a group having an amide bond as described above.

In a case where A in the formula (2) is an organic group having 1 to 30 carbon atoms, A may include an ether oxygen atom. In this case, the ether oxygen atom imparts appropriate flexibility to the fluorine-containing ether compound represented by the formula (1) and increases the affinity between the polar group and the protective layer. In a case where A includes ether oxygen atoms, the number of the ether oxygen atoms is preferably one or two. In this case, it is possible to curb the generation of an intramolecular interaction caused by the fluorine-containing ether compound represented by the formula (1) becoming too flexible by the ether oxygen atoms that is included in A.

In a case where A in the formula (2) is an organic group having 1 to 30 carbon atoms, A may have a halogeno group. In this case, the surface free energy of A in the formula (2) decreases compared with a case where A is an organic group not having a halogeno group. As a result, it is possible to further curb chemical substances being attracted from the environment, and it becomes possible to form a lubricating layer having favorable chemical substance resistance from the fluorine-containing ether compound.

In a case when A includes a halogeno group, the halogeno group is preferably a fluoro group (—F) or a chloro group (—Cl) and more preferably a fluoro group. This is because an effect of decreasing the surface free energy of A is large.

In a case where A in the formula (2) is an organic group having 1 to 30 carbon atoms, A is preferably an acyclic organic group. In a case where A is an acyclic organic group, A does not become too bulky compared with a case where A is an organic group having a cyclic structure. Therefore, it is possible to curb the interaction between the polar group and the protective layer being inhibited by the organic group represented by A hindering the motion of the polar group that is included in the fluorine-containing ether compound. The acyclic organic group may be linear or branched.

In the formula (2), a is an integer of 0 to 2. That is, the terminal group represented by the formula (2) may have a glycerin structure (—O—CH₂—CH(OH)—CH₂—O—) and/or a structure obtained by extending carbon chains in the glycerin structure with (b-1) methylene groups between the internal 1,2-diol structure (—CH₂—CH(OH)—CH(OH)—CH₂—) that is included in the erythritol structure and the methylene group (—CH₂—) adjacent to R².

Since the glycerin structure and the structure obtained by extending carbon chains in the glycerin structure are appropriately rigid, in a case where a is 1 or 2, it is possible to curb the terminal group represented by the formula (2) forming an intramolecular interaction. In addition, since the ether bond possessed by the glycerin structure and the structure obtained by extending carbon chains in the glycerin structure imparts appropriate mobility to the terminal group represented by the formula (2), the hydroxyl groups in the erythritol structure are more likely to get involved in an intermolecular interaction. Therefore, the polar group in the fluorine-containing ether compound represented by the formula (1) is capable of more easily forming an intermolecular interaction. As a result, the lubricating layer containing the fluorine-containing ether compound represented by the formula (1) is less likely to be scattered due to the rotation of magnetic recording media and has superior spinoff resistance.

In the formula (2), b is an integer of 1 to 6 and preferably an integer of 1 to 4. In a case where a is 2, b's that are included in the individual [—CH₂—CH(OH)—(CH₂)_{b}—O—] may be identical to each other or may be different from each other.

The terminal group represented by the formula (2) is preferably any of the following formulae (2-1) to (2-5).

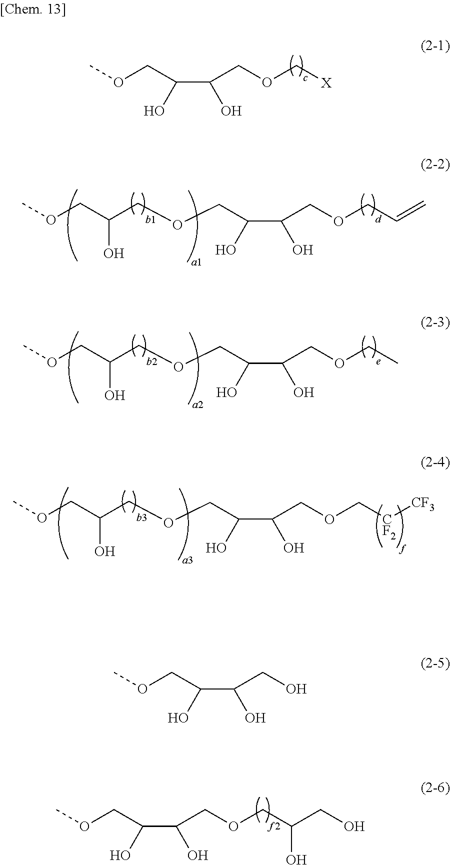

(in the formula (2-1), c represents an integer of 1 to 6. X represents any of a hydroxyl group, an acetamide group, a carboxamide group, and a cyano group.)
(in the formula (2-2), a1 represents 0 or 1. b1 represents an integer of 1 to 6. d represents an integer of 1 to 4.)
(in the formula (2-3), a2 represents 0 or 1. b2 represents an integer of 1 to 6. e represents an integer of 0 to 7.)
(in the formula (2-4), a3 represents 0 or 1. b3 represents an integer of 1 to 6. f represents an integer of 0 to 6.)
(in the formula (2-6), f2 represents an integer of 1 to 6.)

The terminal group represented by the formula (2-1) corresponds to a structure in which a in the formula (2) is 0 and A is —(CH₂)_{c}—X. c represents an integer of 1 to 6. X represents any of a hydroxyl group, an acetamido group, a carboxamide group, and a cyano group.

The terminal group represented by the formula (2-1) has, in addition to the hydroxyl groups in the erythritol structure, a polar group represented by X on the terminal side of the molecule. The polar group represented by X is flexibly mobile and is thus likely to get involved in both the interaction with the protective layer and the interaction between the molecules. As a result, it is possible to sufficiently ensure the number of polar groups that get involved in the interaction between the fluorine-containing ether compound and the protective layer and the interaction between the molecules of the fluorine-containing ether compound, and a lubricating layer having excellent spinoff resistance can be formed.

Since X in the terminal group represented by the formula (2-1) is a hydroxyl group, an acetamido group, a carboxamide group, or a cyano group, it becomes possible to form a lubricating layer having a stronger interaction with the protective layer from the fluorine-containing ether compound. X is preferably a hydroxyl group since the coating state of the fluorine-containing ether compound on the protective layer becomes more uniform and a lubricating layer having more favorable adhesion can be formed.

Since c in the formula (2-1) is 1 or more, the terminal group represented by the formula (2-1) is sufficiently rigid. In addition, since c is 6 or less, the terminal group represented by the formula (2-1) does not become too bulky due to c being too large. Therefore, it is possible to curb the interaction with the protective layer and the interaction between the molecules of the fluorine-containing ether compound being inhibited due to the motion of the hydroxyl groups that are included in the erythritol structure in the terminal group represented by the formula (2-1) being hindered by the c methylene groups. c is preferably 1 to 4 and more preferably 2 to 3.

a1 in the formula (2-2), a2 in the formula (2-3), and a3 in the formula (2-4) correspond to a in the formula (2) and represent 0 or 1.

In addition, b1 in the formula (2-2), b2 in the formula (2-3), and b3 in the formula (2-4) correspond to b in the formula (2) and represent integers of 1 to 6.

a1 in the formula (2-2), a2 in the formula (2-3), and a3 in the formula (2-4) are 0 or 1. Therefore, the number of the hydroxyl groups in the terminal groups represented by the formula (2-2), (2-3), or (2-4) is two or three. Therefore, the fluorine-containing ether compounds having the terminal group represented by the formulae (2-2), (2-3), or (2-4) all enable the formation of a lubricating layer having high chemical substance resistance in which the surface free energy does not become too high due to many polar groups in the compound and a chemical contamination substance is less likely to be incorporated into magnetic recording media.

Since a1, a2, and a3 in the formulae (2-2), (2-3), and (2-4) are 1 or more, the terminal groups represented by the formulae (2-2), (2-3), and (2-4) are sufficiently rigid. In addition, since b1, b2, and b3 are 6 or less, the terminal groups represented by the formulae (2-2), (2-3), and (2-4) do not become too bulky due to b1, b2, and b3 being too large. Therefore, it is possible to curb the interaction with the protective layer and the interaction between the molecules of the fluorine-containing ether compound being inhibited due to the motion of the hydroxyl groups in the terminal groups represented by the formulae (2-2), (2-3), and (2-4) being hindered by the b1, b2, or b3 methylene groups. b1, b2, and b3 are each preferably 1 to 4 and more preferably 1 or 2.

—(CH₂)_{d}—CH═CH₂ in the formula (2-2) corresponds to A in the formula (2). d in the formula (2-2) represents an integer of 1 to 4.

The terminal group represented by the formula (2-2) has a vinyl group on the terminal side of the molecule. The vinyl group is rigid and thus capable of preventing the hydroxyl groups positioned in the vicinity of the vinyl group from incorporating chemical contamination substances, which makes it possible to form a lubricating layer having high chemical substance resistance from the fluorine-containing ether compound.

Since d in the formula (2-2) is 1 or more, the terminal group represented by the formula (2-2) is sufficiently rigid. In addition, since d is 4 or less, the terminal group represented by the formula (2-2) does not become too bulky due to d being too large. Therefore, it is possible to curb the interaction with the protective layer and the interaction between the molecules of the fluorine-containing ether compound being inhibited due to the motion of the hydroxyl groups in the terminal group represented by the formula (2-2) being hindered by the d methylene groups bonding to the rigid vinyl group. d is preferably 1 to 4 and more preferably 1 or 2.

—(CH₂)ₑ—CH₃ in the formula (2-3) corresponds to A in the formula (2). e in the formula (2-3) represents an integer of 0 to 7.

The terminal group represented by the formula (2-3) has a saturated hydrocarbon group on the terminal side of the molecule. An oxygen atom (an oxygen atom in the erythritol structure) adjacent to the saturated hydrocarbon group in the formula (2-3) forms ether bonds with atoms that are bonded to both sides thereof. Since this ether bonds are flexible, the hydroxyl groups in the erythritol structure are considered to be likely to get involved in an intermolecular interaction. As a result, it is possible to sufficiently ensure the number of polar groups that get involved in the interaction between the molecules of the fluorine-containing ether compound, and a lubricating layer having excellent spinoff resistance can be formed.

The terminal group represented by the formula (2-3) is sufficiently rigid even when e is 0. When e is 1 or more, the terminal group has superior rigidity. In addition, since e is 7 or less, the terminal group represented by the formula (2-3) does not become too bulky due to e being too large. Therefore, it is possible to curb the interaction with the protective layer and the interaction between the molecules of the fluorine-containing ether compound being inhibited due to the motion of the hydroxyl groups in the terminal group represented by the formula (2-3) being hindered by the e methylene groups. e is preferably 0 to 4 and more preferably 0 to 2.

—CH₂—(CF₂)_{f}—CF₃ in the formula (2-4) corresponds to A in the formula (2). f in the formula (2-4) represents an integer of 0 to 6.

The terminal group represented by the formula (2-4) has a fluorinated saturated hydrocarbon group on the terminal side of the molecule. The fluorinated saturated hydrocarbon group decreases the surface free energy of the fluorine-containing ether compound. From this fact, the fluorine-containing ether compound having the terminal group represented by the formula (2-4) enables the formation of a lubricating layer having high chemical substance resistance, in which chemical contamination substances are less likely to be incorporated into magnetic recording media.

The terminal group represented by the formula (2-4) is sufficiently rigid even when f is 0. When f is 1 or more, the terminal group has superior rigidity. In addition, since f is 6 or less, the terminal group represented by the formula (2-4) does not become too bulky due to f being too large. Therefore, it is possible to curb the interaction with the protective layer and the interaction between the molecules of the fluorine-containing ether compound being inhibited due to the motion of the hydroxyl groups in the terminal group represented by the formula (2-4) being hindered by the f —CF₂—. f is preferably 0 to 4 and more preferably 0 to 2.

The terminal group represented by the formula (2-5) corresponds to a structure in which a in the formula (2) is 0 and A is a hydrogen atom (the hydrogen atom bonds to an adjacent oxygen atom to form a hydroxyl group).

The terminal group represented by the formula (2-5) has, in addition to the two hydroxyl groups in the internal 1,2-diol structure that is included in the erythritol structure, a hydroxyl group at the outermost terminal. That is, the terminal group represented by the formula (2-5) has a 1,2,3-triol structure.

The 1,2,3-triol structure is less likely to form an intramolecular interaction since the motion thereof is controlled by steric repulsion. Therefore, the hydroxyl groups in the terminal group represented by the formula (2-5) get involved in both the interaction with the protective layer and the interaction between the molecules. As a result, the fluorine-containing ether compound having the terminal group represented by the formula (2-5) sufficiently ensures the number of polar groups that get involved in the interaction with the protective layer and the interaction between the molecules of the fluorine-containing ether compound, and enables the formation of a lubricating layer having excellent spinoff resistance.

The terminal group represented by the formula (2-6) corresponds to a structure in which a in the formula (2) is 0 and A is —(CH₂)_{f2}—CH(OH)CH₂OH. f2 represents an integer of 1 to 6.

The terminal group represented by the formula (2-6) has, in addition to the hydroxyl groups in the erythritol structure, two hydroxyl groups on the terminal side of the molecule. These hydroxyl groups are flexibly mobile and thus likely to get involved in both the interaction with the protective layer and the interaction between the molecules. As a result, it is possible to sufficiently ensure the number of polar groups that get involved in the interaction between the fluorine-containing ether compound and the protective layer and the interaction between the molecules of the fluorine-containing ether compound, and a lubricating layer having excellent spinoff resistance can be formed. In addition, since these hydroxyl groups are 1,2-diol structures like the hydroxyl groups in the erythritol structure, all of the hydroxyl groups are capable of uniformly interacting with each other. As a result, the coating state of the fluorine-containing ether compound on the protective layer becomes more uniform, and a lubricating layer having more favorable adhesion can be formed.

Since f2 in the formula (2-6) is 1 or more, the terminal group represented by the formula (2-6) is sufficiently rigid. In addition, since f2 is 6 or less, the terminal group represented by the formula (2-6) does not become too bulky due to f2 being too large. Therefore, it is possible to curb the interaction with the protective layer and the interaction between the molecules of the fluorine-containing ether compound being inhibited due to the motion of the hydroxyl groups that are included in the erythritol structure in the terminal group represented by the formula (2-1) being hindered by the f2 methylene groups. f2 is preferably 1 to 4, more preferably 1 or 2, and most preferably 1.

In the fluorine-containing ether compound represented by the formula (1), R¹ and R⁴ may be identical to each other or may be different from each other. R¹ and R⁴ are preferably identical to each other. In this case, the fluorine-containing ether compound can be easily and efficiently produced.

“R¹ and R⁴ are identical to each other” means that the atoms that are included in R¹ and the atoms that are included in R⁴ are symmetrically disposed with respect to —CH₂—R²[—CH₂—R³—CH₂—R²]ₓ—CH₂—.

In the fluorine-containing ether compound represented by the formula (1), in a case where R¹ and R⁴ are different from each other, R¹ and R⁴ may be each independently the terminal group represented by the formula (2) or only one of R¹ and R⁴ may be the terminal group represented by the formula (2) and the other may be a terminal group that does not correspond to the formula (2). The terminal group that does not correspond to the formula (2) needs to be a terminal group having 1 to 4 polar groups and having 1 to 50 carbon atoms as described above.

### Terminal Group Represented by the Formula (3)

In a case where only one of R¹ and R⁴ is the terminal group represented by the formula (2), and the other is a terminal group that does not correspond to the formula (2), the terminal group that does not correspond to the formula (2) is preferably represented by the following formula (3).

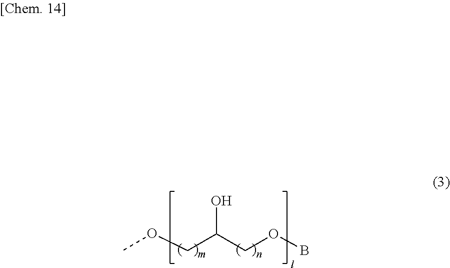

(in the formula (3), 1 represents an integer of 1 to 3. l m's each independently represent an integer of 1 to 6. l n's each independently represent an integer of 1 to 6. In one repeating unit, at least one of m and n is 1. B represents an alkyl group optionally having only one polar group, an organic group including a carbon-carbon unsaturated bond optionally having only one polar group, or a hydrogen atom.)

The terminal group represented by the formula (3) has an oxygen atom (ether oxygen atom) that is bonded to the methylene group (—CH₂—) bonding to R². The oxygen atom disposed at the end portion of the terminal group represented by the formula (3) forms an ether bond (—O—) with atoms that are bonded to both sides thereof. This ether bond imparts appropriate flexibility to the fluorine-containing ether compound represented by the formula (1) and increases the affinity between the polar group in the terminal group represented by the formula (3) and the protective layer. This makes the fluorine-containing ether compound represented by the formula (1) enable the formation of a lubricating layer having excellent adhesion to the protective layer.

l in the formula (3) is an integer of 1 to 3, preferably an integer of 1 or 2, and more preferably 1. When l in the formula (3) is 3 or less, it is possible to prevent chemical substances in the environment from being attracted to the lubricating layer due to an excessively large number of the hydroxyl groups in the terminal group represented by the formula (3), and a lubricating layer having favorable chemical substance resistance can be obtained.

In a case where l in the formula (3) is 2 or 3, the combinations of m and n in the two or three repeating units (—(CH₂)ₘ—CH(OH)—(CH₂)ₙ—O—) may be each different from each other or may be partially or fully identical to each other.

l m's in the formula (3) each independently represent an integer of 1 to 6. l n's each independently represent an integer of 1 to 6. In one repeating unit ((—(CH₂)ₘ—CH(OH)—(CH₂)ₙ—O—) in the formula (3), at least one of m and n is 1. In this case, it is possible to curb the mobility of the hydroxyl group in the repeating unit being decreased due to an excessively large number of carbon atoms in the alkylene group between the carbon atom bonding to the hydroxyl group and the ether oxygen atom.

B in the formula (3) represents an alkyl group optionally having one polar group, an organic group including a carbon-carbon unsaturated bond optionally having only one polar group, or a hydrogen atom.

In a case where B in the formula (3) is an alkyl group having no polar groups, examples of B include a methyl group, an ethyl group, a propyl group, a butyl group, a pentyl group, a hexyl group, and the like.

In a case where B in the formula (3) is an alkyl group having a polar group, the polar group is preferably a polar group that have been exemplified as the preferable examples of the polar group that is included in R¹ and R⁴. Among the above-described polar groups, a polar group selected from the group consisting of a hydroxyl group, a cyano group, and a group having an amide bond is more preferable.

In a case where B in the formula (3) is an alkyl group having a polar group, examples of B include a 2-hydroxyethyl group, a 3-hydroxypropyl group, a 4-hydroxybutyl group, a 5-hydroxypentyl group, a 6-hydroxyhexyl group, a 2-aminoethyl group, a 3-aminopropyl group, a 1-carboxymethyl group, a 2-carboxyethyl group, a 3-carboxypropyl group, a 1-carbonylmethyl group, a 2-carbonylethyl group, a 3-carbonylpropyl group, a 1-acetylmethyl group, a 2-acetylethyl group, a 3-acetylpropyl group, a 2-sulfoethyl group, a 3-sulfopropyl group, a 1-cyanomethyl group, a 2-cyanoethyl group, a 3-cyanopropyl group, a 4-cyanobutyl group, a 2-acetamidoethyl group, a 3-acetamidopropyl group, a 4-acetamidobutyl group, a 1-carboxamidomethyl group, a 2-carboxamidoethyl group, a 3-carboxamidopropyl group, a 4-carboxamidobutyl group, and the like.

Among the above-described alkyl groups having a polar group, any of a 2-hydroxyethyl group, a 3-hydroxypropyl group, a 4-hydroxybutyl group, a 5-hydroxypentyl group, a 6-hydroxyhexyl group, a 1-cyanomethyl group, a 2-cyanoethyl group, a 3-cyanopropyl group, a 2-acetamidoethyl group, a 1-carboxamidomethyl group, a 2-carboxamidoethyl group, or a 3-carboxamidopropyl is preferable, and any of a 2-hydroxyethyl group, a 3-hydroxypropyl group, a 2-cyanoethyl group, a 3-cyanopropyl group, a 2-acetamidoethyl group, or a 1-carboxamidomethyl group is more preferable.

In a case where B in the formula (3) is an organic group including a carbon-carbon unsaturated bond optionally having only one polar group, examples of B include organic groups including at least one selected from an aromatic hydrocarbon, an unsaturated heterocycle, an alkenyl group, and an alkynyl group.

In a case where B in the formula (3) is the organic group including a carbon-carbon unsaturated bond optionally having only one polar group, examples of B include a phenyl group, a methoxyphenyl group, a fluorinated phenyl group, an acetamidophenyl group, a carboxamidophenyl group, a cyanophenyl group, a naphthyl group, a phenethyl group, a methoxyphenethyl group, a fluorinated phenethyl group, a benzyl group, a methoxybenzyl group, a naphthylmethyl group, a methoxynaphthyl group, a pyrrolyl group, a pyrazolyl group, a methylpyrazolylmethyl group, an imidazolyl group, a furyl group, a furfuryl group, an oxazolyl group, an isoxazolyl group, a thienyl group, a thienylethyl group, a thiazolyl group, a methyl thiazolylethyl group, an isothiazolyl group, a pyridyl group, a pyrimidinyl group, a pyridazinyl group, a pyrazinyl group, an indolinyl group, a benzofuranyl group, a benzothienyl group, a benzimidazolyl group, a benzoxazolyl group, a benzothiazolyl group, a benzopyrazolyl group, a benzisoxazolyl group, a benzisothiazolyl group, a quinolyl group, an isoquinolyl group, a quinazolinyl group, a quinoxalinyl group, a phthalazinyl group, a cinnolinyl group, a vinyl group, an allyl group, a butenyl group, a propynyl group, a propargyl group, a butynyl group, a methylbutynyl group, a pentynyl group, a methylpentynyl group, a hexynyl group, and the like.

Among the above-described organic group including a carbon-carbon unsaturated bond optionally having only one polar group, any of a phenyl group, a methoxyphenyl group, an acetamidophenyl group, a carboxamidophenyl group, a cyanophenyl group, a naphthyl group, a phenethyl group, a methoxyphenethyl group, a fluorinated phenethyl group, a thienylethyl group, an allyl group, a butenyl group, or a propargyl group is preferable, and in particular, any of a phenyl group, a methoxyphenyl group, a carboxamidophenyl group, an allyl group, or a butenyl group is more preferable. In this case, the terminal group represented by the formula (3) is sufficiently rigid, does not become too bulky, and is capable of curbing the interaction with the protective layer being inhibited due to the motion of the hydroxyl group in the formula (3) being hindered.

In a case where B in the formula (3) is a hydrogen atom, B forms a hydroxyl group with an oxygen atom in the formula (3).

The terminal group represented by the formula (3) is more preferably represented by any of the following formulae (3-1) to (3-3).

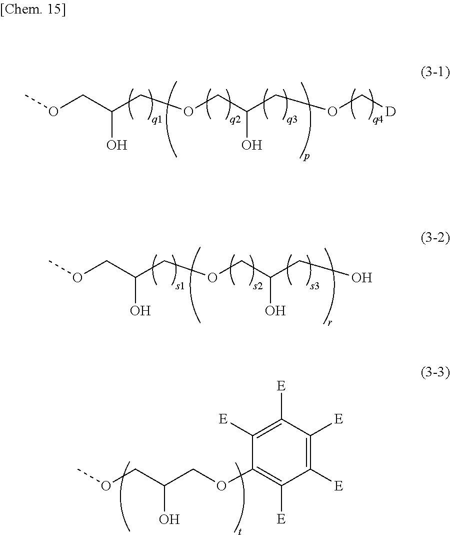

(in the formula (3-1), p represents 0 or 1. q1, q2, q3, and q4 each independently represent an integer of 1 to 6. In a case where p is 0, a total value of q1 and q4 is 2 to 10. In a case where p is 1, a total value of q1, q2, q3, and q4 is 4 to 10. In a case where p is 1, at least one of q2 and q3 is 1. D represents a polar group, a vinyl group, an ethynyl group, or an aryl group optionally having a substituent. Here, in a case where D is an aryl group optionally having a substituent, the number of polar groups included in D is 0 or 1.)
(in the formula (3-2), r represents 0 or 1. s1, s2, and s3 each independently represent an integer of 1 to 6. In a case where r is 1, a total value of s1, s2, and s3 is 3 to 8. In a case where r is 1, at least one of s2 and s3 is 1.)
(in the formula (3-3), t represents 1 or 2. Five E's each independently represent a polar group, an alkoxy group having 1 to 8 carbon atoms, a halogeno group, or a hydrogen atom. Here, in a case where a polar group is included in the five E's, a total number of the polar groups in the five E's is one.)

To the terminal group represented by the formula (3-1), one or two glycerin structures (—O—CH₂—CH(OH)—CH₂—O—) and/or a structure obtained by extending carbon chains in the glycerin structure with a methylene group is linked, and D bonds to the terminal thereof through at least one methylene group. The glycerin structure and the structure obtained by extending carbon chains in the glycerin structure are appropriately rigid and thus capable of curbing the hydroxyl groups that are included in the terminal group represented by the formula (3-1) forming an intramolecular interaction. In addition, an ether bond in the glycerin structure and the structure obtained by extending carbon chains in the glycerin structure impart appropriate mobility to the terminal group represented by the formula (3-1). Therefore, the hydroxyl groups that are included in the terminal group represented by the formula (3-1) are likely to get involved in the interaction with the protective layer or the interaction between the molecules of the fluorine-containing ether compound. As a result, a lubricating layer having excellent chemical substance resistance and spinoff resistance can be formed.

D in the formula (3-1) represents a polar group, a vinyl group, an ethynyl group, or an aryl group optionally having a substituent. In a case where D is a polar group, C is preferably a polar group that have been exemplified as the preferable examples of the polar group that is included in Ri and R⁴. Among the above-described polar groups, a polar group selected from the group consisting of a hydroxyl group, a cyano group, and a group having an amide bond is more preferable. In a case where D is an aryl group optionally having a substituent, B in the formula (3) is preferably an aryl group optionally having a substituent that is included an organic group that can be used in a case where B is an organic group including a carbon-carbon unsaturated bond optionally having only one polar group.

In the terminal group represented by the formula (3-1), since the total value of q1 and q4 in a case where p is 0 and the total value of q1, q2, q3, and q4 in a case where p is 1 are 10 or less, and an alkylene chain in the main chain portion is not too long. Therefore, it is possible to prevent deterioration of the flexibility of the terminal portion of the fluorine-containing ether compound, weakening of the interaction between the hydroxyl groups that are included in the terminal group represented by the formula (3-1) and the protective layer, and the floating of the terminal portion from the protective layer all caused by the rigid alkylene chain disposed in the main chain portion of the terminal group represented by the formula (3-1) being long. Therefore, it is possible to prevent the lubricating layer containing the fluorine-containing ether compound from being less likely to uniformly coat the entire surface of the protective layer due to an excessively large number of carbon atoms that are included in the terminal group represented by the formula (3-1).

In the terminal group represented by the formula (3-1), since the total value of q1 and q4 in a case where p is 0 is 2 or more, and the total value of q1, q2, q3, and q4 in a case where p is 1 is 4 or more, the number of carbon atoms in the main chain portion of the formula (3-1) is not too small. Therefore, it is possible to prevent the fluorine-containing ether compound from aggregating due to the polarity of the terminal group represented by the formula (3-1) being too high.

q1, q2, q3, and q4 are each an integer of 1 to 6, preferably 1 to 3, and more preferably 1 or 2. At least one of q1, q2, q3, and q4 is preferably 2 or more.

To the terminal group represented by the formula (3-2), one or two glycerin structures (—O—CH₂—CH(OH)—CH₂—O—) and/or a structure obtained by extending carbon chains in the glycerin structure with a methylene group is linked, and a hydroxyl group bonds to the terminal thereof. The glycerin structure and the structure obtained by extending carbon chains in the glycerin structure are appropriately rigid and thus capable of curbing the hydroxyl groups that are included in the terminal group represented by the formula (3-2) forming an intramolecular interaction. In addition, an ether bond in the glycerin structure and the structure obtained by extending carbon chains in the glycerin structure imparts appropriate mobility to the terminal group represented by the formula (3-2). Therefore, the hydroxyl groups that are included in the terminal group represented by the formula (3-2) are likely to get involved in the interaction with the protective layer or the interaction between the molecules of the fluorine-containing ether compound. As a result, a lubricating layer having excellent chemical substance resistance and spinoff resistance can be formed.

In the terminal group represented by the formula (3-2), since s1 in a case where r is 0 is 6 or less, and the total value of s1, s2, and s3 in a case where r is 1 is 8 or less, the alkylene chain in the main chain portion is not too long. Therefore, it is possible to prevent deterioration of the flexibility of the terminal portion of the fluorine-containing ether compound, weakening of the interaction between the hydroxyl groups that are included in the terminal group represented by the formula (3-2) and the protective layer, and the floating of the terminal portion all caused by the rigid alkylene chain disposed in the main chain portion of the terminal group represented by the formula (3-2) being long. Therefore, it is possible to prevent the lubricating layer containing the fluorine-containing ether compound from being less likely to uniformly coat the entire surface of the protective layer due to an excessively large number of carbon atoms that are included in the terminal group represented by the formula (3-2).

In the terminal group represented by the formula (3-2), since s1 is 1 or more in a case where r is 0 (a case where there are two hydroxyl groups), and the total value of s1, s2, and s3 is 3 or more in a case where r is 1 (a case where there are three hydroxyl groups), the number of carbon atoms in the main chain portion of the formula (3-2) is not too small. Therefore, it is possible to prevent the fluorine-containing ether compound from aggregating due to the polarity of the terminal group represented by the formula (3-2) being too high.

s1, s2, and s3 are each an integer of 1 to 6, preferably 1 to 3, and more preferably 1 or 2. At least one of s1, s2, and s3 is preferably 2 or more.

The terminal group represented by the formula (3-3) has an aryl group at the terminal of one glycerin structure (—O—CH₂—CH(OH)—CH₂—O—) or at the terminal of two linked glycerin structures. The glycerin structure is appropriately rigid. Therefore, the glycerin structure is capable of curbing the hydroxyl groups that are included in the terminal group represented by the formula (3-3) forming an intramolecular interaction. In addition, an ether bond in the glycerin structure imparts appropriate mobility to the terminal group represented by the formula (3-3). Therefore, the hydroxyl groups that are included in the terminal group represented by the formula (3-3) are likely to get involved in the interaction with the protective layer or the interaction between the molecules of the fluorine-containing ether compound. As a result, a lubricating layer having excellent chemical substance resistance and spinoff resistance can be formed.

In the terminal group represented by the formula (3-3), five E's each independently represent a polar group, an alkoxy group having 1 to 8 carbon atoms, a halogeno group, or a hydrogen atom. In a case where a polar group is included in the five E's, the total number of the polar groups in the five E's is one.

In the terminal group represented by the formula (3-3), in a case where a polar group is included in the five E's, the polar group bonds to a benzene ring that is rigid and difficult to freely rotate, which curbs the hydroxyl groups that are included in the glycerin structure in the formula (3-3) and the polar group that is included in E forming an intramolecular interaction. Therefore, in a case where a polar group is included in the five E's, the fluorine-containing ether compound having the terminal group represented by the formula (3-3) has a large number of polar groups capable of getting involved in the intermolecular interaction. As a result, a lubricating layer containing the fluorine-containing ether compound having the terminal group represented by the formula (3-3) has a high spinoff-curbing effect.

In the terminal group represented by the formula (3-3), in a case where no polar groups are included in the five E's, the surface free energy of the fluorine-containing ether compound becomes low compared with a case where one of the five E's is a polar group. As a result, the lubricating layer containing the fluorine-containing ether compound having the terminal group represented by the formula (3-3) is capable of curbing chemical substances being attracted from the environment and has higher chemical substance resistance.

In the terminal group represented by the formula (3-3), in a case where E is a polar group, E is preferably a polar group that have been exemplified as the preferable examples of the polar group that is included in Ri and R⁴. Among the above-described polar groups, E is more preferably a polar group selected from the group consisting of a cyano group and a group having an amide bond and still more preferably a cyano group, an acetamide group (—NHC(═O)CH₃), or a carboxamide group (—C(═O)NH₂). This is because when the polar group is a cyano group, an acetamide group, or a carboxamide group, it is possible to form a lubricating layer having a stronger interaction with the protective layer from the fluorine-containing ether compound. In addition, since the acidity of the cyano group, the acetamide group, or the carboxamide group is not too high, the fluorine-containing ether compounds having this group rarely corrodes substrates. In a case where E is not a polar group, Es' are each independently preferably a methoxy group, a fluoro group, or a hydrogen atom.

In a case where one of the five E's in the formula (3-3) is a polar group, the four E's other than the polar group may be partially or fully identical to one another or may be each different from one another. In a case where a polar group is included in the five E's, four E's other than the polar group are all preferably identical to one another. In a case where one of the five E's is a polar group, the position of the polar group may be at the position of any of the five E's.

In a case where no polar groups are included in the five E's in the formula (3-3), the five E's may be partially or fully identical to one another or may be each different from one another.

Terminal Groups that does not Correspond to the Formula (3)

In a case where only one of R¹ and R⁴ is the terminal group represented by the formula (2), and the other is a terminal group that does not correspond to the formula (2), the terminal group that does not correspond to the formula (2) may be a terminal group that does not correspond to the formula (3). Examples of the terminal group that does not correspond to the formula (3) include terminal groups represented by the following formula (3-4) or (3-5).

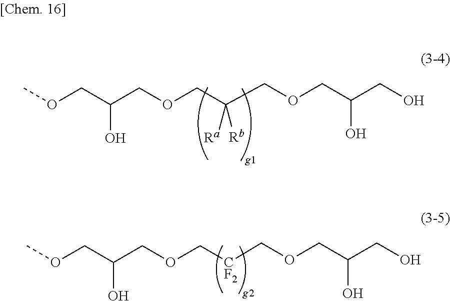

(in the formula (3-4), g1 represents an integer of 1 to 6. g1 R^{a}'s and g1 R^{b}'s are each independently a hydrogen atom or a methyl group.)
(in the formula (3-5), g2 represents an integer of 1 to 6.)

g1 —CR^{a}R^{b}-'s in the terminal group represented by the formula (3-4) may be each any of —CH₂—, —CH(CH₃)—, or —C(CH₃)₂—.

In the terminal group represented by the formula (3-4), in a case where R^{a} and R^{b} disposed between the two glycerin structures are hydrogen atoms, the terminal group does not become too bulky compared with a case where R^{a} and/or R^{b} is a methyl group. Therefore, it is possible to curb the interaction of the hydroxyl groups with the protective layer being inhibited due to the motion of the hydroxyl groups in the terminal group being hindered. As a result, when one of the two hydroxyl groups that form a terminal 1,2-diol structure has interacted with the protective layer, the other hydroxyl group is capable of more easily forming an intermolecular interaction with a polar group in another fluorine-containing ether compound. Therefore, in the lubricating layer containing the fluorine-containing ether compound represented by the formula (1), the scattering of the fluorine-containing ether compound associated with the rotation of magnetic recording media is far less likely to occur, and the lubricating layer has superior spinoff resistance.

In the terminal group represented by the formula (3-4), in a case where the organic group disposed between the two glycerin structures includes —CH(CH₃)— and/or —C(CH₃)₂—, the organic group becomes appropriately rigid and is capable of effectively curbing the intramolecular interaction between the hydroxyl groups in the terminal group. Therefore, the polar groups in the fluorine-containing ether compound represented by the formula (1) is capable of more easily forming an intermolecular interaction. Therefore, in the lubricating layer containing the fluorine-containing ether compound represented by the formula (1), the scattering of the fluorine-containing ether compound associated with the rotation of magnetic recording media is far less likely to occur, and the lubricating layer has superior spinoff resistance.

Since g1 in the formula (3-4) is 1 or more, the terminal group represented by the formula (3-4) is appropriately rigid. In addition, since g1 is 6 or less, the terminal group represented by the formula (3-4) does not become too bulky, and it is possible to curb the interaction with the protective layer being inhibited due to the motion of the hydroxyl groups in the terminal group being hindered. g1 is preferably 1 to 4 and more preferably 1 or 2.

In the terminal group represented by the formula (3-5), the organic group between the two glycerin structures is —CH₂—(CF₂)g₂-CH₂—. g2 represents an integer of 1 to 6.

The terminal group represented by the formula (3-5) includes a linear perfluoroalkylene chain having 1 to 6 carbon atoms, which reduces affinity to chemical substances in the environment. As a result, the lubricating layer containing the fluorine-containing ether compound represented by the formula (1) has favorable chemical substance resistance.

Since g2 in the formula (3-5) is 1 or more, the terminal group represented by the formula (3-5) becomes appropriately rigid and is capable of curbing an intramolecular interaction. In addition, since g2 is 6 or less, the terminal group represented by the formula (3-5) does not become too bulky, and it is possible to curb the interaction with the protective layer being inhibited due to the motion of the hydroxyl groups being hindered. g2 in the formula (3-5) is preferably 1 to 4 and more preferably 2 to 4.

### Divalent Linking Group Indicated by R³

In a case where x in the fluorine-containing ether compound represented by the formula (1) is 1 or 2, the fluorine-containing ether compound includes x R³ groups. R³ is disposed between adjacent PFPE chains through a methylene group. R³ is a divalent linking group having 1 to 4 polar groups and makes the fluorine-containing ether compound closely adhere to the protective layer.

In the formula (1), in a case where x is 2, two R³'s may be identical to each other or may be each different from each other, but are preferably identical to each other. When the two R³'s are identical to each other, the coating state of the fluorine-containing ether compound on the protective layer becomes more uniform, and a lubricating layer having more favorable adhesion can be formed. In addition, when the two R³'s are identical to each other, the fluorine-containing ether compound can be easily and efficiently produced.

In the present specification, “two R³'s are identical to each other” in a case where x is 2 means that the atoms that are included in the two R³'s are symmetrically disposed with respect to R² disposed in the center of the chain structure of the molecule.

x R³'s in the formula (1) each include one or more polar groups. Therefore, in a case where a lubricating layer has been formed on the protective layer using a lubricant containing the fluorine-containing ether compound with x of 1 or 2, a suitable interaction is generated between the x R³'s and the protective layer. Therefore, the fluorine-containing ether compound with x of 1 or 2 enables the formation of a lubricating layer having excellent adhesion to the protective layer and a high spinoff-curbing effect compared with the fluorine-containing ether compound with x of 0.

In addition, since the number of the polar groups that are included in R³ is four or less, it is possible to prevent a chemical substance in the environment from being incorporated into magnetic recording media due to the polarity of the fluorine-containing ether compound with x of 1 or 2 become too high. As a result, it becomes possible to form a lubricating layer having high chemical substance resistance from the fluorine-containing ether compound. The number of the polar groups that are included in R³ is preferably three or less.

The polar group that is included in R³ is preferably at least one polar group selected from the group consisting of a hydroxyl group (—OH), a carboxy group (—COOH), a formyl group (—(C═O)H), a carbonyl group (—(C═O)R⁷; R⁷ is an organic group), a sulfo group (—SO₃H), a cyano group (—CN), a group having an amide bond (—NR⁸COR⁹ or —CONR¹⁰R¹¹; R⁸, R⁹, R¹⁰, and R¹¹ are each independently a hydrogen atom or an organic group), and an amino group (—NR¹²R¹³; R¹² and R¹³ are each independently a hydrogen atom or an organic group).

The group having an amide bond includes both a group that bonds at a carbon atom configuring the amide bond (for example, a carboxamide group (—C(═O)NH₂)) and a group that bonds at a nitrogen atom configuring the amide bond (for example, an acetamido group (—NHC(═O)CH₃)) as shown in the above-described formula. In the group having an amide bond, R⁸ and R⁹ may bond to each other to form a ring, and R¹⁰ and R¹¹ may bond to each other to form a ring. R⁸, R⁹, R¹⁰, and R¹¹ in the group having an amide bond are each independently preferably selected from the group consisting of a hydrogen atom, a methyl group, an ethyl group, a propyl group, and a butyl group.

R³ preferably includes at least one polar group selected from the group consisting of a hydroxyl group, a cyano group, and a group having an amide bond. This is because the hydroxyl group, the cyano group, and the group having an amide bond are chemically stable and a lubricating layer containing the fluorine-containing ether compound having these polar groups does not alter for a long period of time. In addition, this is because the hydroxyl group, the cyano group, and the group having an amide bond have an acidity that is not too high and are less likely to corrode substrates.

The one to four polar groups in each of the x R³'s in the formula (1) may be partially or fully identical to one another or may be each different from one another.

The x R³'s in the formula (1) each preferably includes at least one hydroxyl group, and all of the polar groups in the x R³'s are more preferably hydroxyl groups. This is because the coating state of the fluorine-containing ether compound on the protective layer becomes more uniform.

x R³'s in the formula (1) are each preferably a linking group having 1 to 50 carbon atoms, more preferably a linking group having 3 to 50 carbon atoms, still more preferably a linking group having 3 to 20 carbon atoms, and most preferably a linking group having 4 to 15 carbon atoms. When the number of carbon atoms in the linking group represented by R³ is one or more, the rigidity of the linking group can be secured. Therefore, it is possible to prevent the polar group that is included in the linking group represented by R³ from attracting chemical substances in the environment into the lubricating layer, and a lubricating layer having favorable chemical substance resistance can be formed. When the number of carbon atoms in the linking group represented by R³ is 50 or less, the linking group becomes a flexible structure, and the adhesion between the lubricating layer containing the fluorine-containing ether compound and the protective layer becomes favorable. As a result, a lubricating layer capable of curbing spinoff can be obtained.

x R³'s in the formula (1) are each preferably a divalent linking group having oxygen atoms disposed at both end portions. In this case, the oxygen atoms disposed at both end portions of the divalent linking group represented by R³ form ether bonds (—O—) with the methylene groups (—CH₂—) disposed on both sides of R³. These ether bonds impart appropriate flexibility to the fluorine-containing ether compound represented by the formula (1) and increase the affinity between the polar groups in the divalent linking group indicated by R³ and the protective layer.

x R³'s in the formula (1) are each independently more preferably a divalent linking group having 1 to 3 hydroxyl groups, having oxygen atoms at both end portions bonding to the adjacent methylene groups, and having 3 to 50 carbon atoms.

More specifically, x R³'s are each independently preferably any one selected from linking groups represented by the following formulae (4-1) to (4-6).

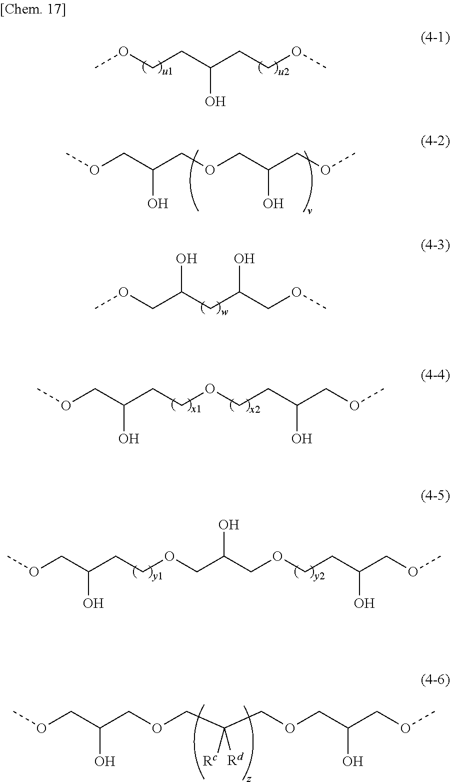

(in the formula (4-1), u1 represents an integer of 0 to 6, and u2 represents an integer of 0 to 6. Here, at least one of u1 and u2 is 0. An oxygen atom at a left terminal of the formula (4-1) bonds to a methylene group on an R¹ side in the formula (1), and an oxygen atom at a right terminal bonds to a methylene group on an R⁴ side in the formula (1).)
(in the formula (4-2), v represents an integer of 1 or 2. An oxygen atom at a left terminal of the formula (4-2) bonds to the methylene group on the R¹ side in the formula (1), and an oxygen atom at a right terminal bonds to the methylene group on the R⁴ side in the formula (1).)
(in the formula (4-3), w represents an integer of 0 to 6. An oxygen atom at a left terminal of the formula (4-3) bonds to the methylene group on the R¹ side in the formula (1), and an oxygen atom at a right terminal bonds to the methylene group on the R⁴ side in the formula (1).)
(in the formula (4-4), x1 represents an integer of 0 to 5, and x2 represents an integer of 0 to 5. Here, at least one of x1 and x2 is an integer of 1 to 5. An oxygen atom at a left terminal of the formula (4-4) bonds to the methylene group on the R¹ side in the formula (1), and an oxygen atom at a right terminal bonds to the methylene group on the R⁴ side in the formula (1).)
(in the formula (4-5), y1 represents an integer of 1 to 5, and y2 represents an integer of 1 to 5. An oxygen atom at a left terminal of the formula (4-5) bonds to the methylene group on the R¹ side in the formula (1), and an oxygen atom at a right terminal bonds to the methylene group on the R⁴ side in the formula (1).)
(in the formula (4-6), z represents an integer of 1 to 6. z R^{c}'s and z R^{d}'s are each independently a hydrogen atom, a fluorine atom, or a methyl group. An oxygen atom at a left terminal of the formula (4-6) bonds to the methylene group on the R¹ side in the formula (1), and an oxygen atom at a right terminal bonds to the methylene group on the R⁴ side in the formula (1).)

The linking group indicated by the formula (4-1) has a glycerin structure (—O—CH₂—CH(OH)—CH₂—O—) or a structure obtained by adding one to six methylene groups to the glycerin structure. The linking group indicated by the formula (4-1) includes only one hydroxyl group, which keeps the polarity of the linking group low. As a result, it is possible to effectively prevent the intrusion of chemical substances in the environment, and a lubricating layer having high chemical substance resistance can be formed.

In the formula (4-1), since at least one of u1 and u2 is 0, the fluorine-containing ether compound having this linking group has excellent flexibility, and the coating state of the fluorine-containing ether compound on the protective layer becomes more uniform. In a case where only one of u1 and u2 is 0, the value of any one of u1 and u2 that is not 0 is 1 to 6, preferably 1 to 4, and more preferably 1 to 3. This is because the linking group does not become too rigid and a lubricating layer having more favorable adhesion and excellent spinoff resistance can be formed.

In addition, in the formula (4-1), in a case where only one of u1 and u2 is 0, the linking group has u1 or u2 carbon atoms that bond neither the polar group nor the ether oxygen atom between the carbon atom bonding to the secondary hydroxyl group and the ether oxygen atoms forming the ether bonds disposed at both ends. Therefore, the linking group has excellent rigidity attributed to the u1 and u2 methylene groups, it is possible to prevent the polar group in the linking group represented by R³ from attracting chemical substances in the environment to the lubricating layer, and it becomes possible to form a lubricating layer having more favorable chemical substance resistance from the fluorine-containing ether compound.

The linking group indicated by the formula (4-2) has a structure in which two or three glycerin structures (—O—CH₂—CH(OH)—CH₂—O—) are linked to one another. Since the glycerin structure imparts flexibility to the linking group, the linking group indicated by the formula (4-2) is extremely flexible. As a result, the fluorine-containing ether compound having this linking group enables the formation of a lubricating layer having more favorable adhesion and superior spinoff resistance.

In the formula (4-2), v represents an integer of 1 or 2 and is preferably 1. Since v is 2 or less, the polarity of the fluorine-containing ether compound represented by the formula (1) is kept low, and a lubricating layer having superior chemical substance resistance can be formed.

The linking group indicated by the formula (4-3) has a structure in which the carbon atoms to which the hydroxyl groups bond have directly bonded to each other or a structure in which the carbon atoms have bonded to each other through an alkylene group including no ether bonds and having 1 to 6 carbon atoms. Therefore, the linking group indicated by the formula (4-3) has a polarity kept low compared with, for example, a linking group having a structure in which carbon atoms to which hydroxyl groups have bonded have bonded to each other through an oxygen atom that forms an ether bond and an alkylene group. As a result, the intrusion of chemical substances in the environment can be effectively inhibited, and a lubricating layer having high chemical substance resistance can be formed.

In the formula (4-3), w represents an integer of 0 to 6. When w is 6 or less, the linking group indicated by the formula (4-3) does not become too rigid, and it becomes possible to form a lubricating layer having more favorable adhesion and excellent spinoff resistance from the fluorine-containing ether compound. w is preferably 4 or less.

In addition, in the formula (4-3), in a case where w is 1 or more, the linking group has w carbon atoms that do not bond to any of the polar group and the ether oxygen atom between the two carbon atoms to which secondary hydroxyl groups have bonded. Therefore, the linking group has excellent rigidity attributed to the w methylene groups, and it becomes possible to form a lubricating layer having more favorable chemical substance resistance from the fluorine-containing ether compound. In a case where w is 1 or more, w is preferably 2 to 4.

The linking group indicated by the formula (4-4) has a structure in which the carbon atoms to which the hydroxyl groups have bonded have bonded to each other through a linear linking chain of 4 or more atoms including oxygen atoms that have formed the ether bonds. In the fluorine-containing ether compound having the linking group indicated by the formula (4-4), since the distance between the two hydroxyl groups in the formula (4-4) is sufficiently maintained, the intramolecular interaction between the hydroxyl groups is curbed, and a lubricating layer having favorable adsorption capability onto the protective layer can be formed. Therefore, the lubricating layer containing the fluorine-containing ether compound having this linking group has more favorable adhesion and superior spinoff resistance.

In the formula (4-4), x1 and x2 each independently represent an integer of 0 to 5, and at least one of x1 and x2 is an integer of 1 to 5. In the linking group indicated by the formula (4-4), since the total value of x1 and x2 is 1 or more, the fluorine-containing ether compound becomes appropriately rigid, and a lubricating layer containing this fluorine-containing ether compound is less likely to allow the incorporation of chemical substances in the environment and has favorable chemical substance resistance. x1 and x2 are each independently preferably an integer of 3 or less. This is because the linking group does not become too rigid and enables the formation of a lubricating layer having more favorable adhesion and excellent spinoff resistance. In addition, the total value of x1 and x2 is preferably 4 or less. One of x1 and x2 is preferably 0 since appropriate flexibility is imparted to the linking group represented by the formula (4-4).

The linking group indicated by the formula (4-5) has a structure in which three hydroxyl groups are present, and carbon atoms bonding to adjacent hydroxyl groups have bonded to each other through a linear linking chain of 4 or more atoms including oxygen atoms that have formed ether bonds.

In the fluorine-containing ether compound having the linking group indicated by the formula (4-5), since the linking group indicated by the formula (4-5) has three hydroxyl groups, an interaction between the hydroxyl group and the protective layer is easily formed, and it becomes possible to form a lubricating layer having favorable adhesion to the protective layer. Furthermore, in the fluorine-containing ether compound having the linking group indicated by the formula (4-5), since the distances between adjacent hydroxyl groups of the three hydroxyl groups in the formula (4-5) are sufficiently maintained, the intramolecular interaction between the hydroxyl groups is curbed, and a lubricating layer having favorable adsorption capability onto the protective layer can be formed. Therefore, the lubricating layer containing the fluorine-containing ether compound having this linking group has more favorable adhesion and superior spinoff resistance.

In the formula (4-5), y1 and y2 each independently represent an integer of 1 to 5. In the linking group indicated by the formula (4-5), since y1 and y2 are both 1 or more, the fluorine-containing ether compound becomes appropriately rigid, and the lubricating layer containing this linking group is less likely to allow the incorporation of chemical substances in the environment and has favorable chemical substance resistance. y1 and y2 are each independently preferably 1 to 3, and both are most preferably 1 since appropriate flexibility is imparted to the linking group represented by the formula (4-5). In addition, y1 and y2 are preferably identical to each other since the coating state of the fluorine-containing ether compound on the protective layer becomes more uniform.

The linking group indicated by the formula (4-6) has a structure in which carbon atoms to which hydroxyl groups have bonded have bonded to each other through a linking chain of 7 or more atoms including two ether bonds. In the linking group indicated by the formula (4-6), since the distance between the two hydroxyl groups is sufficiently maintained, the intramolecular interaction between the hydroxyl groups is curbed. Therefore, the linking group indicated by the formula (4-6) has favorable adsorption capability onto the protective layer. As a result, the fluorine-containing ether compound having the linking group indicated by the formula (4-6) has more favorable adhesion and superior spinoff resistance.

In the formula (4-6), z represents an integer of 1 to 6. z is preferably 4 or less since appropriate flexibility is imparted to the linking group represented by the formula (4-6). In addition, z is preferably 2 or more since sufficient rigidity is imparted to the linking group represented by the formula (4-6).

z R^{c}'s and z R^{d}'s in the formula (4-6) each independently represent a hydrogen atom, a fluorine atom, or a methyl group. z (—CR^{c}R^{d}—)'s are each preferably any of —CH₂—, —CH(CH₃)—, —C(CH₃)₂—, or —CF₂— since the production becomes easy.

In a case where the z (—CR^{c}R^{d}—)'s are only one or a plurality of —CH₂—'s, the linking group indicated by the formula (4-6) becomes flexible compared with a case where, for example, —CH(CH₃)— and/or —C(CH₃)₂— are included, which is preferable.

In addition, in a case where at least some of the z (—CR^{c}R^{d}—)'s include —CH(CH₃)— and/or —C(CH₃)₂—, the linking group becomes appropriately bulky compared with a case where the z (—CR^{c}R^{d}—)'s are all —CH₂—, and the intramolecular interactions between the hydroxyl groups in the linking group indicated by the formula (4-6) can be effectively curbed, which is preferable.

In addition, in a case where the z (—CR^{c}R^{d}—)'s include one or a plurality of —CF₂-'s, the surface free energy of the linking group indicated by the formula (4-6) decreases, and the linking group is far less likely to incorporate chemical substances in the environment, which is preferable.

### PFPE Chain Indicated by R²

In the fluorine-containing ether compound represented by the formula (1), R² is a perfluoropolyether chain. The PFPE chain indicated by R² coats the surface of a protective layer and imparts lubricity to a lubricating layer to reduce a friction force between a magnetic head and the protective layer in a case where a lubricant containing the fluorine-containing ether compound of the present embodiment is applied onto the protective layer to form the lubricating layer. The PFPE chain indicated by R² is selected as appropriate depending on performances or the like that are required for the lubricant containing the fluorine-containing ether compound.

Examples of the PFPE chain indicated by R² include PFPE chains composed of a polymer or copolymer of a perfluoroalkylene oxide. Examples of the perfluoroalkylene oxide include perfluoromethylene oxide, perfluoroethylene oxide, perfluoro-n-propylene oxide, perfluoroisopropylene oxide, perfluorobutylene oxide, and the like.

In a case where x in the formula (1) is 1 or 2, (x+1) R²'s may be partially or fully identical to each other or may be different from each other. R²'s in the formula (1) is each independently preferably, for example, a PFPE chain represented by the following formula (5) derived from a polymer or copolymer of a perfluoroalkylene oxide.

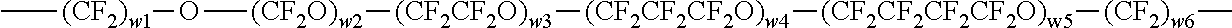

(in the formula (5), w2, w3, w4, and w5 indicate the average degrees of polymerization and each independently represent 0 to 20. Here, w2, w3, w4, and w5 are not all 0 at the same time. w1 and w6 are average values representing the numbers of CF₂'s and each independently represent 1 to 3. An array order of (CF₂O), (CF₂CF₂O), (CF₂CF₂CF₂O), and (CF₂CF₂CF₂CF₂O), which are repeating units in the formula (5), is not particularly limited.)

In the formula (5), w2, w3, w4, and w5 indicate the average degrees of polymerization, each independently represent 0 to 20, and are preferably 0 to 15 and more preferably 0 to 10.

In the formula (5), w1 and w6 are average values indicating the numbers of CF₂'s and each independently represent 1 to 3. w1 and w6 are determined depending on the structure of the repeating unit that is disposed at the end portion of the chain structure in the PFPE chain represented by the formula (5).

(CF₂O), (CF₂CF₂O), (CF₂CF₂CF₂O), and (CF₂CF₂CF₂CF₂) in the formula (5) are repeating units. The array order of the repeating units in the formula (5) is particularly limited. In addition, the number of the kinds of the repeating units in the formula (5) is also particularly limited.

R²'s in the formula (1) are each independently preferably any one selected from the PFPE chains represented by the following formulae (5-1) to (5-4).

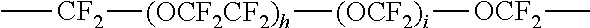

(in the formula (5-1), h and i indicate average degrees of polymerization, h represents 1 to 20, and i represents 0 to 20.)

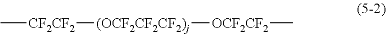

(in the formula (5-2), j indicates an average degree of polymerization and represents 1 to 15.)

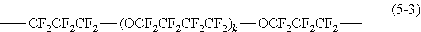

(in the formula (5-3), k indicates an average degree of polymerization and represents 1 to 10.)

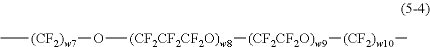

(in the formula (5-4), w8 and w9 indicate average degrees of polymerization and each independently represent 1 to 20. w7 and w10 are average values representing the numbers of CF₂'s and each independently represent 1 or 2.)

When R² is any one selected from the PFPE chains represented by the formulae (5-1) to (5-4), it becomes possible to obtain a lubricating layer having favorable lubricity from the fluorine-containing ether compound. In addition, in a case where R² is any one selected from the PFPE chains represented by the formulae (5-1) to (5-4), the proportion of the number of oxygen atoms (the number of ether bonds (—O—)) in the number of carbon atoms in the PFPE chain is appropriate. Therefore, the fluorine-containing ether compound has an appropriate hardness. Therefore, the fluorine-containing ether compound applied onto the protective layer is less likely to aggregate on the protective layer, and it is possible to form a lubricating layer of thinner thickness with a sufficient coating rate. In addition, a lubricating layer containing the fluorine-containing ether compound in which R² is any one selected from the PFPE chains represented by the formulae (5-1) to (5-4) becomes denser and is capable of curbing spinoff, which is preferable.

In the formula (5-1), the array order of (OCF₂CF₂) and (OCF₂), which are the repeating units, is not particularly limited. In the formula (5-1), the number h of (OCF₂CF₂)'s and the number i of (OCF₂)'s may be identical to each other or may be different from each other. The PFPE chain represented by the formula (5-1) may be a polymer of (OCF₂CF₂). In addition, the PFPE chain represented by the formula (5-1) may be any of a random copolymer, block copolymer, or alternating copolymer composed of (OCF₂CF₂) and (OCF₂).

In the formulae (5-1) to (5-3), since h is 1 to 20, i is 0 to 20, j is 1 to 15, and k is 1 to 10, h, i, j, and k indicating the average degrees of polymerization, it becomes possible to obtain a lubricating layer having favorable lubricity from the fluorine-containing ether compound. In addition, in the formulae (5-1) to (5-3), since h and i are 20 or less, j is 15 or less, and k is 10 or less, h, i, j, and k indicating the average degrees of polymerization, the viscosity of the fluorine-containing ether compound does not become too high, and a lubricant containing this fluorine-containing ether compound becomes easy to apply, which is preferable. h, i, j, and k indicating the average degrees of polymerization are preferably 1 to 10, more preferably 1.5 to 8, and still more preferably 2 to 7 since the fluorine-containing ether compound easily spreads on the protective layer, and it becomes possible to form a lubricating layer having a uniform film thickness from the fluorine-containing ether compound.

In the formula (5-4), the array order of (CF₂CF₂CF₂O) and (CF₂CF₂O), which are the repeating units, is not particularly limited. In the formula (5-4), the number w8 of (CF₂CF₂CF₂O)'s and the number w9 of (CF₂CF₂O)'s, w8 and w9 indicating the average degrees of polymerization, may be identical to each other or may be different from each other. The formula (5-4) may include any of a random copolymer, block copolymer, or alternating copolymer composed of the monomer units (CF₂CF₂CF₂O) and (CF₂CF₂O).

In the formula (5-4), w8 and w9 indicating the average degrees of polymerization are each independently 1 to 20, preferably 1 to 15, and more preferably 1 to 10. w7 and w10 in the formula (5-4) are average values indicating the number of CF₂'s and each independently represents 1 or 2. w7 and w10 are determined depending on the structure of the repeating units that are disposed at the end portion of the chain structure of the PFPE chain represented by the formula (5-4).

The fluorine-containing ether compound represented by the formula (1) is, specifically, preferably any of the compounds represented by the following the formulae (AA) to (AX), (BA) to (BP), (CA) to (CM), and (DA). In a case where the compound represented by the formula (1) is any of the compounds represented by the following formulae (AA) to (AX), (BA) to (BP), (CA) to (CM), and (DA)), a raw material is easy to obtain, and furthermore, it is possible to form a lubricating layer having more favorable chemical substance resistance and being capable of curbing spinoff even when the thickness is thin.

In the compounds represented by the following the formulae (AA) to (AX), (BA) to (BP), (CA) to (CM), and (DA), Rf₁, Rf₂, and Rf₃ representing the PFPE chains are each the following structure. That is, in the compounds represented by the following formulae (AA), (AD) to (AI), (AK) to (AN), (AP), (AR), (AX), (BA), (BE), (BN) to (BP), (CA) to (CI), (CL), and (CM), Rf₁ is the PFPE chain represented by the formula (5-1). In the compounds represented by the formulae (AB), (AJ), (AO), (AQ), (AS) to (AW), (BB) to (BD), (BF) to (BM), (CJ), and (DA), Rf₂ is the PFPE chain represented by the formula (5-2). In the compounds represented by the formulae (AC) and (CK), Rf₃ is the PFPE chain represented by the formula (5-3). h and i in Rf₁, j in Rf₂, and k in Rf₃, Rf₁, Rf₂, and Rf₃ representing the PFPE chains in the formulae (AA) to (AX), (BA) to (BP), (CA) to (CM), and (DA), are values indicating the average degrees of polymerization and thus do not necessarily become an integer.

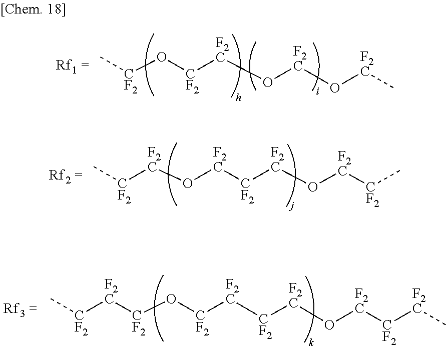

In the compounds represented by the following the formulae (AA) to (BM) and (DA), x in the formula (1) is 0. In the compounds represented by the formulae (AA) to (AX), R¹ and R⁴ are identical to each other. In the compounds represented by the formulae (AA) to (AH), R¹ and R⁴ are the terminal group represented by the formula (2-1).

In the compounds represented by the formulae (AI) to (AM), R¹ and R⁴ are the terminal group represented by the formula (2-2).

In the compounds represented by the formulae (AN) to (AR), R¹ and R⁴ are the terminal group represented by the formula (2-3).

In the compounds represented by the formulae (AS) to (AV), R¹ and R⁴ are the terminal group represented by the formula (2-4).

In the compound represented by the formula (AW), R¹ and R⁴ are the terminal group represented by the formula (2-5).

In the compound represented by the formula (AX), R¹ and R⁴ are the terminal group represented by the formula (2), and A is an organic group including one ether oxygen atom and having three carbon atoms.

In the compounds represented by the formulae (BA) to (BC), R¹ and R⁴ are different from each other and are each a terminal group represented by any of the formulae (2-1) to (2-3).

In the compounds represented by the formulae (BD) to (BL), R¹ and R⁴ are different from each other, one is a terminal group represented by any of the formulae (2-1) and (2-2), and the other is a terminal group represented by any of the formulae (3-1) and (3-2).

In the compound represented by the formula (BM), R¹ and R⁴ are different from each other, one is a terminal group represented by the formula (2-2), and the other is a terminal group represented by the formula (3-4).

In the compounds represented by the formulae (BN) and (BO), R¹ and R⁴ are different from each other, one is a terminal group represented by the formula (2-1), and the other is a terminal group represented by the formula (3-3). In the compound represented by the formula (BN), one of the five E's in the formula (3-3) is a carboxamide group (—C(═O)NH₂). In the compound represented by the formula (BO), one of the five E's in the formula (3-3) is a methoxy group.

In the compound represented by the formula (BP), R¹ and R⁴ are different from each other, one is a terminal group represented by the formula (2-1). The other is a terminal group represented by the formula (3-5) and including a perfluoroalkylene chain having two carbon atoms.

In the compounds represented by the formulae (CA) to (CK), x in the formula (1) is 1, and R¹ and R⁴ are identical to each other. In the compounds represented by the formulae (CA) to (CE), Ri and R⁴ are terminal groups represented by any of the formulae (2-1) to (2-5). R³ is the linking group represented by the formula (4-1).

In the compounds represented by the formulae (CF) to (CI), R¹ and R⁴ are the terminal group represented by the formula (2-1). R³ is a linking group represented by any of the formulae (4-2) to (4-5).

In the compounds represented by the following formulae (CJ) and (CK), Ri and R⁴ are the terminal groups represented by the formula (2-1). R³ is the linking group represented by the formula (4-1).

In the compound represented by the formula (CL), x in the formula (1) is 2. R¹ and R⁴ are identical to each other and are the terminal group represented by the formula (2-1). The two R³ groups are the linking groups represented by the formula (4-1).

In the compound represented by the formula (CM), x in the formula (1) is 1. R¹ and R⁴ are identical to each other and are the terminal groups represented by the formula (2-1). R³ is the linking group represented by the formula (4-6).

In the compound represented by the formula (DA), x in the formula (1) is 0. R¹ and R⁴ are different from each other, one is the terminal group represented by the formula (2-6), and the other is the terminal group represented by the formula (3-2).

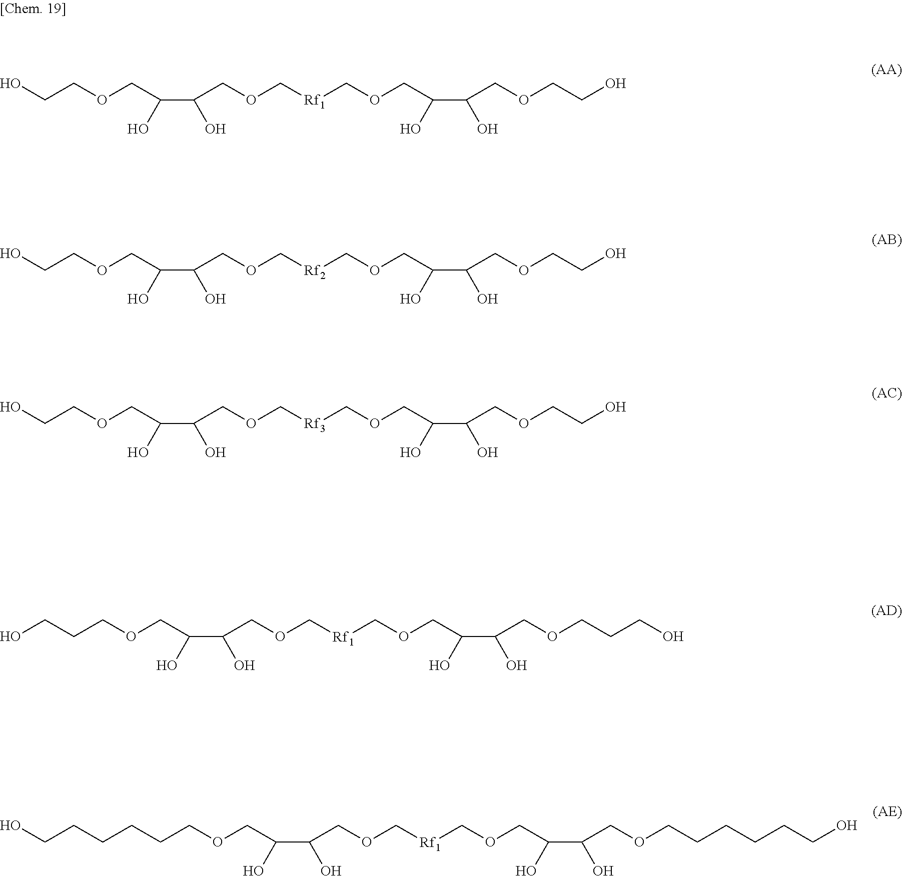

(in Rf₁ in the formula (AA), h and i indicate average degrees of polymerization, h represents 1 to 20, and i represents 0 to 20.)
(in Rf₂ in the formula (AB), j indicates an average degree of polymerization and represents 1 to 15.)
(in Rf₃ in the formula (AC), k indicates an average degree of polymerization and represents 1 to 10.)
(in Rf₁ in the formula (AD), h and i indicate average degrees of polymerization, h represents 1 to 20, and i represents 0 to 20.)
(in Rf₁ in the formula (AE), h and i indicate average degrees of polymerization, h represents 1 to 20, and i represents 0 to 20.)

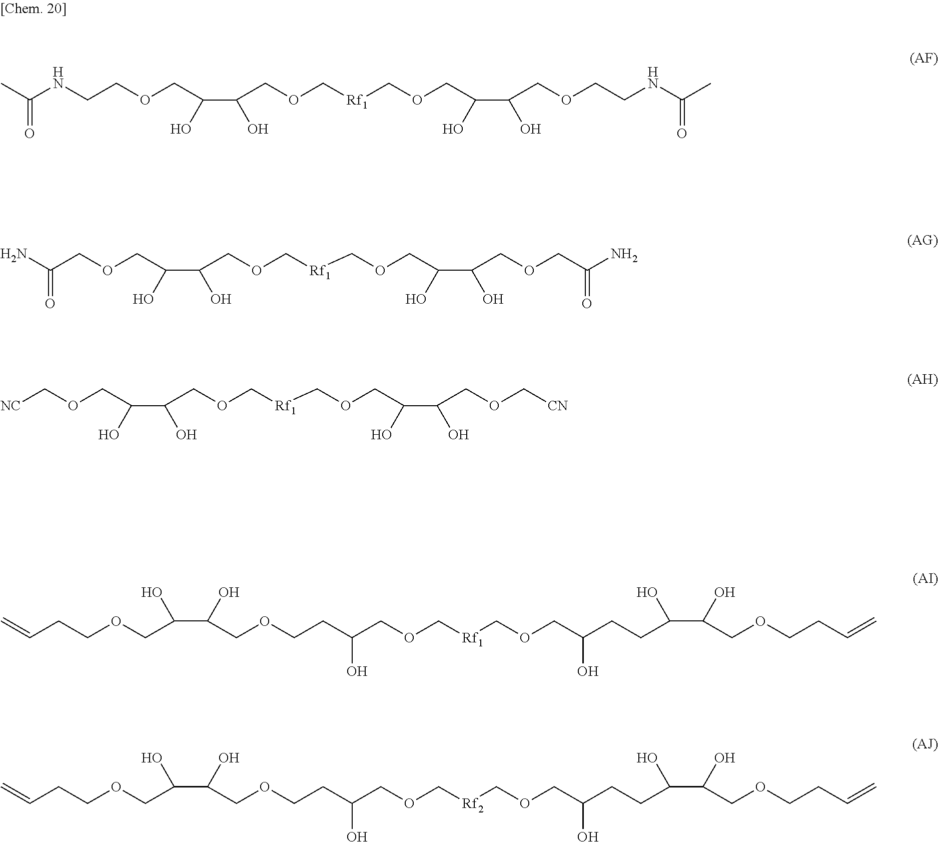

(in Rf₁ in the formula (AF), h and i indicate average degrees of polymerization, h represents 1 to 20, and i represents 0 to 20.)
(in Rf₁ in the formula (AG), h and i indicate average degrees of polymerization, h represents 1 to 20. and i represents 0 to 20.)
(in Rf₁ in the formula (AH), h and i indicate average degrees of polymerization, h represents 1 to 20, and i represents 0 to 20.)
(in Rf₁ in the formula (AI), h and i indicate average degrees of polymerization, h represents 1 to 20, and i represents 0 to 20.)
(in Rf₂ in the formula (AJ), j indicates an average degree of polymerization and represents 1 to 15.)

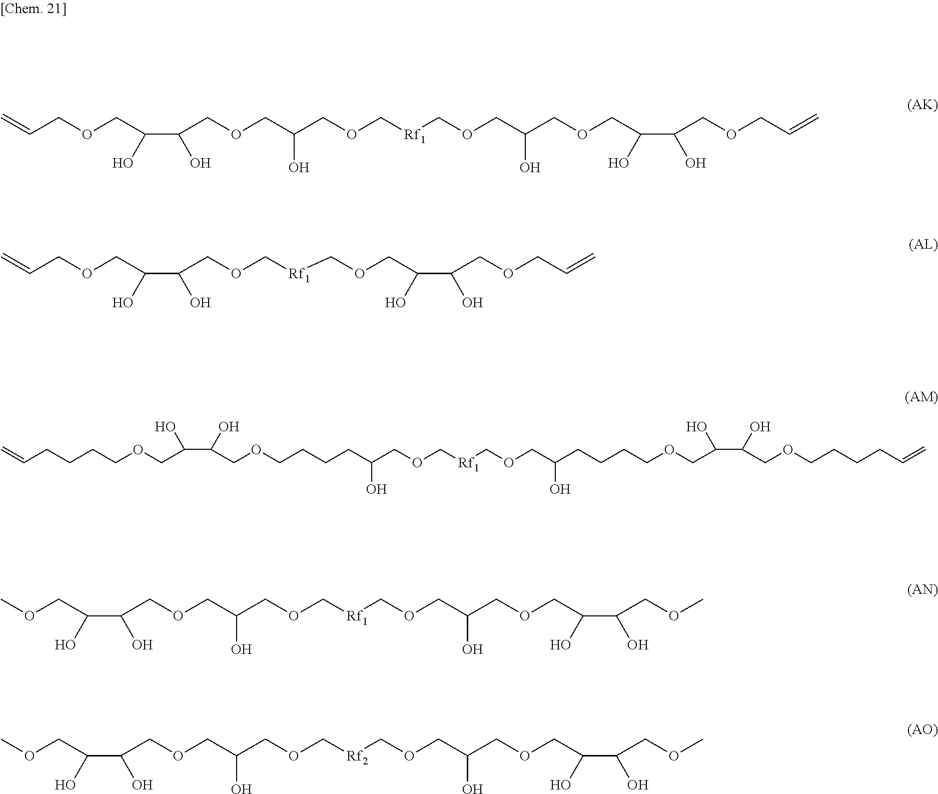

(in Rf₁ in the formula (AK), h and i indicate average degrees of polymerization, h represents 1 to 20, and i represents 0 to 20.)
(in Rf₁ in the formula (AL), h and i indicate average degrees of polymerization, h represents 1 to 20, and i represents 0 to 20.)
(in Rf₁ in the formula (AM), h and i indicate average degrees of polymerization, h represents 1 to 20, and i represents 0 to 20.)
(in Rf₁ in the formula (AN), h and i indicate average degrees of polymerization, h represents 1 to 20, and i represents 0 to 20.)
(in Rf₂ in the formula (AO), j indicates an average degree of polymerization and represents 1 to 15.)

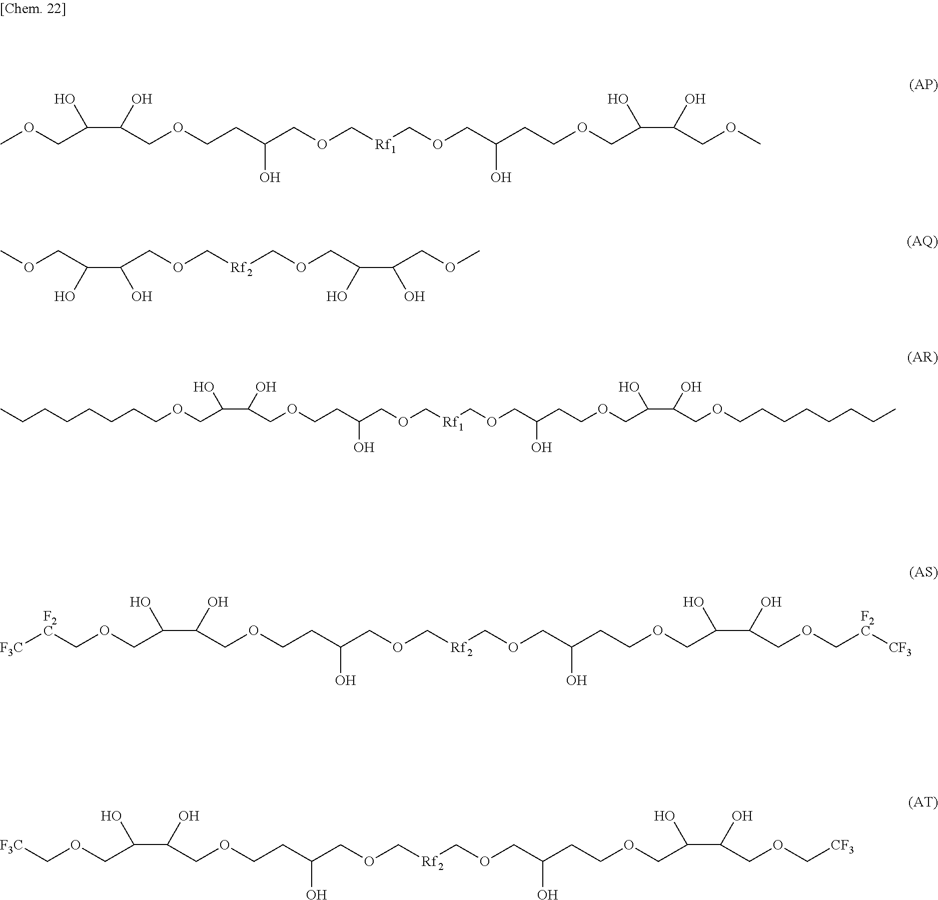

(in Rf₁ in the formula (AP), h and i indicate average degrees of polymerization, h represents 1 to 20, and i represents 0 to 20.)
(in Rf₂ in the formula (AQ), j indicates an average degree of polymerization and represents 1 to 15.)
(in Rf₁ in the formula (AR), h and i indicate average degrees of polymerization, h represents 1 to 20, and i represents 0 to 20.)
(in Rf₂ in the formula (AS), j indicates an average degree of polymerization and represents 1 to 15.)
(in Rf₂ in the formula (AT), j indicates an average degree of polymerization and represents 1 to 15.)

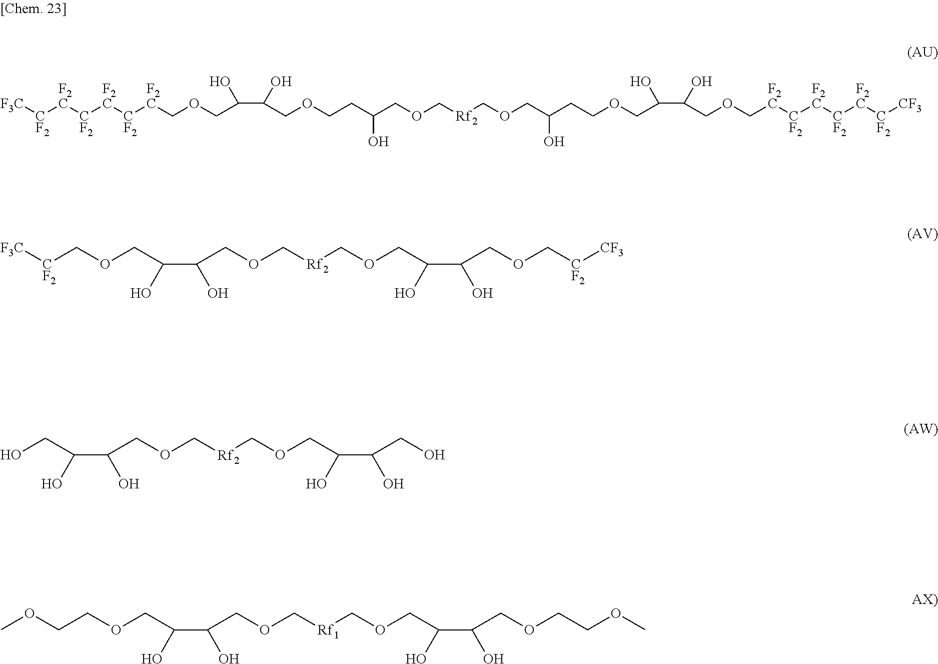

(in Rf₂ in the formula (AU), j indicates an average degree of polymerization and represents 1 to 15.)
(in Rf₂ in the formula (AV), j indicates an average degree of polymerization and represents 1 to 15.)
(in Rf₂ in the formula (AW), j indicates an average degree of polymerization and represents 1 to 15.)
(in Rf₁ in the formula (AX), h and i indicate average degrees of polymerization, h represents 1 to 20, and i represents 0 to 20.)

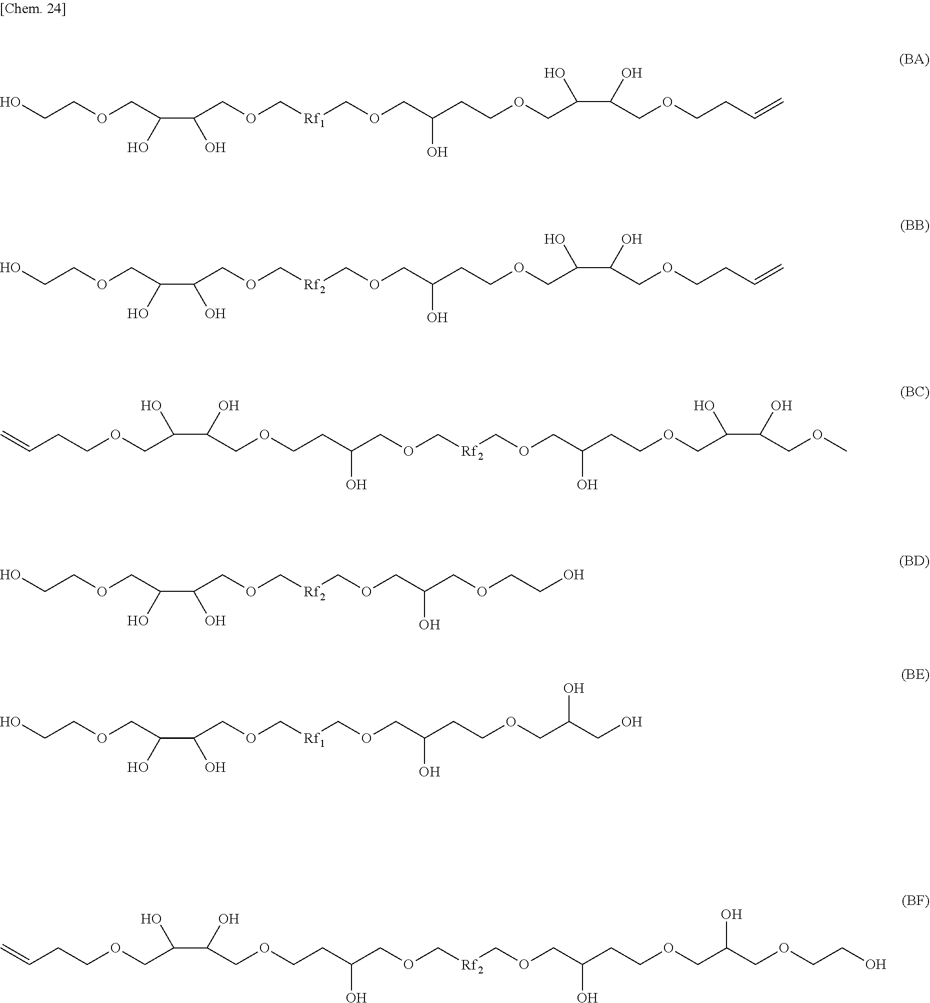

(in Rf₁ in the formula (BA), h and i indicate average degrees of polymerization, h represents 1 to 20, and i represents 0 to 20.)
(in Rf₂ in the formula (BB), j indicates an average degree of polymerization and represents 1 to 15.)
(in Rf₂ in the formula (BC), j indicates an average degree of polymerization and represents 1 to 15.)
(in Rf₂ in the formula (BD), j indicates an average degree of polymerization and represents 1 to 15.)
(in Rf₁ in the formula (BE), h and i indicate average degrees of polymerization, h represents 1 to 20, and i represents 0 to 20.)
(in Rf₂ in the formula (BF), j indicates an average degree of polymerization and represents 1 to 15.)

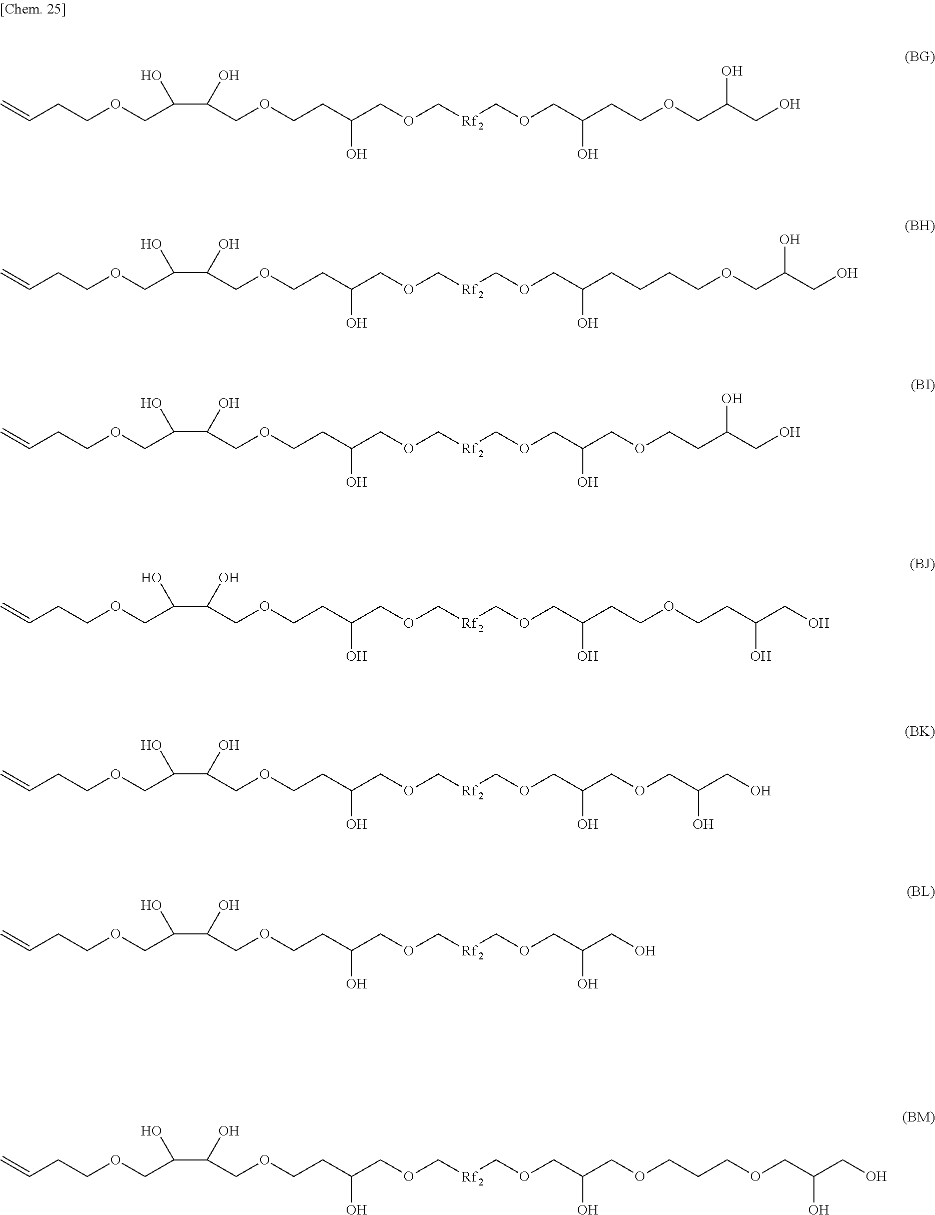

(in Rf₂ in the formula (BG), j indicates an average degree of polymerization and represents 1 to 15.)
(in Rf₂ in the formula (BH), j indicates an average degree of polymerization and represents 1 to 15.)
(in Rf₂ in the formula (BI), j indicates an average degree of polymerization and represents 1 to 15.)
(in Rf₂ in the formula (BJ), j indicates an average degree of polymerization and represents 1 to 15.)
(in Rf₂ in the formula (BK), j indicates an average degree of polymerization and represents 1 to 15.)
(in Rf₂ in the formula (BL), j indicates an average degree of polymerization and represents 1 to 15.)
(in Rf₂ in the formula (BM), j indicates an average degree of polymerization and represents 1 to 15.)

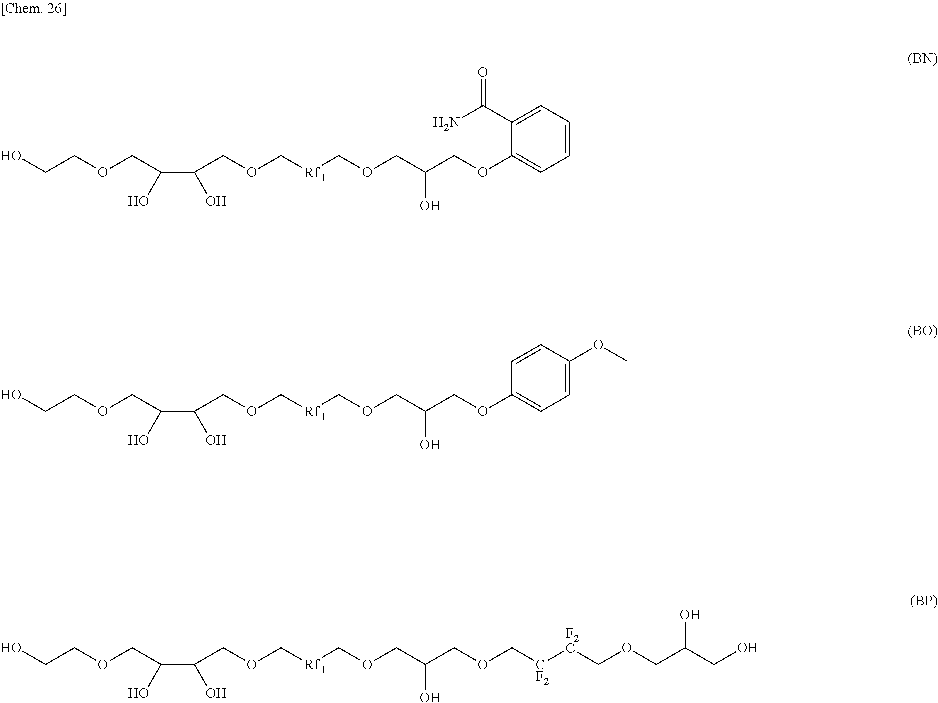

(in Rf₁ in the formula (BN), h and i indicate average degrees of polymerization, h represents 1 to 20, and i represents 0 to 20.)
(in Rf₁ in the formula (BO), h and i indicate average degrees of polymerization, h represents 1 to 20, and i represents 0 to 20.)
(in Rf₁ in the formula (BP), h and i indicate average degrees of polymerization, h represents 1 to 20, and i represents 0 to 20.)

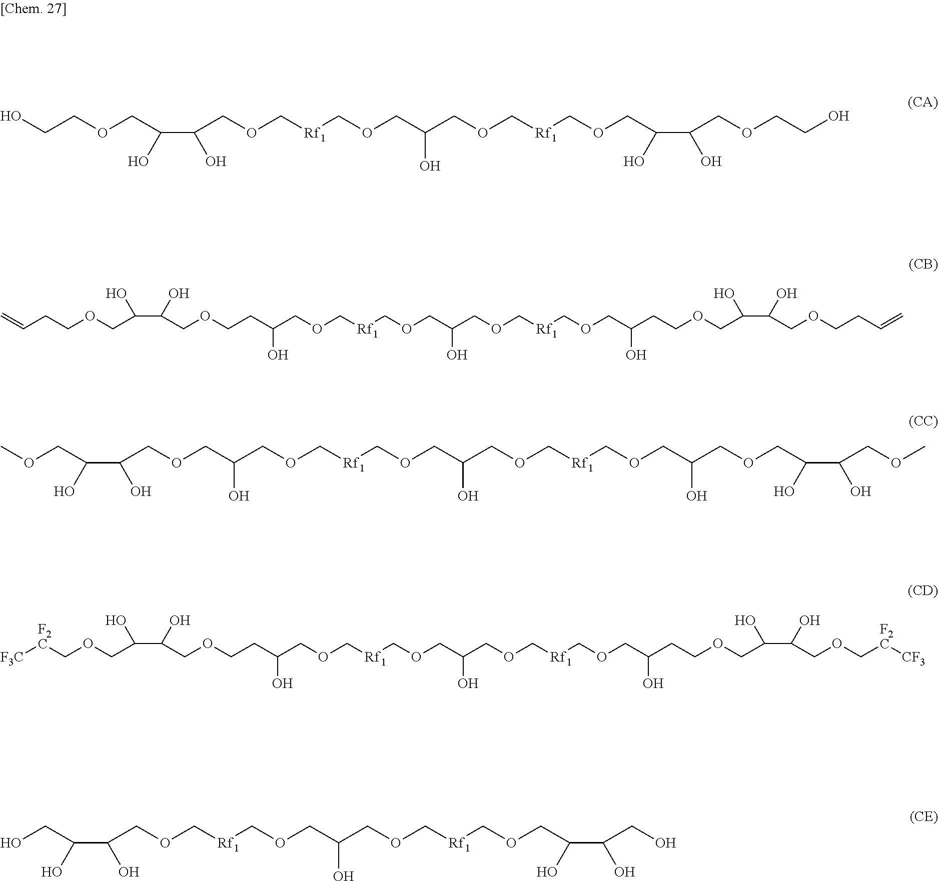

(in two Rf₁'s in the formula (CA), h and i indicate average degrees of polymerization, h represents 1 to 20, and i represents 0 to 20. h's and i's in the two Rf₁'s may be identical to each other or may be different from each other.)
(in two Rf₁'s in the formula (CB), h and i indicate average degrees of polymerization, h represents 1 to 20, and i represents 0 to 20. h's and i's in the two Rf₁'s may be identical to each other or may be different from each other.)
(in two Rf₁'s in the formula (CC), h and i indicate average degrees of polymerization, h represents 1 to 20, and i represents 0 to 20. h's and i's in the two Rf₁'s may be identical to each other or may be different from each other.)
(in two Rf₁'s in the formula (CD), h and i indicate average degrees of polymerization, h represents 1 to 20, and i represents 0 to 20. h's and i's in the two Rf₁'s may be identical to each other or may be different from each other.)
(in two Rf₁'s in the formula (CE), h and i indicate average degrees of polymerization, h represents 1 to 20, and i represents 0 to 20. h's and i's in the two Rf₁'s may be identical to each other or may be different from each other.)

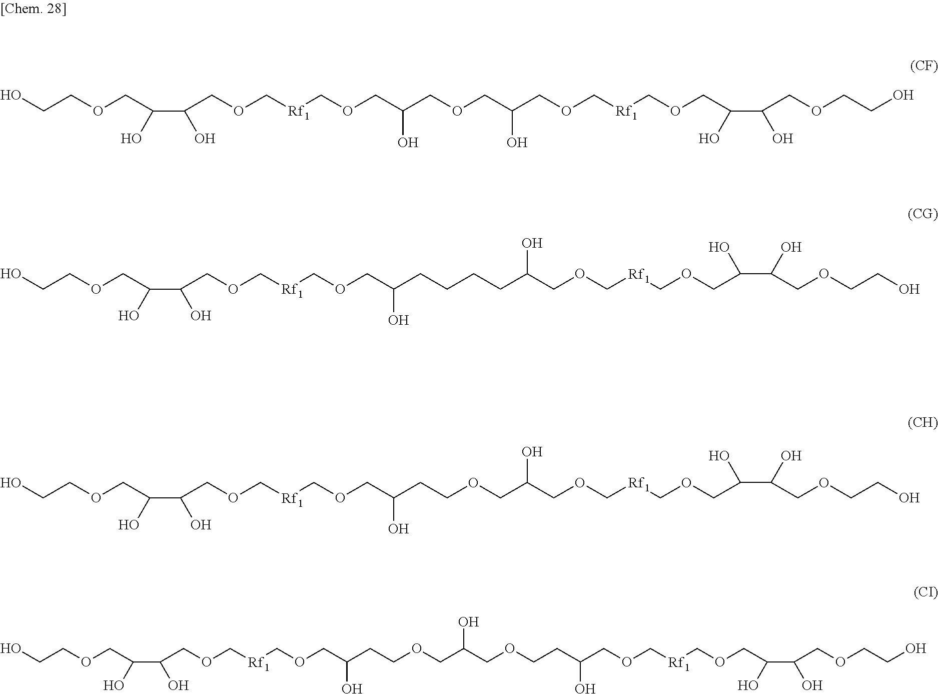

(in two Rf₁'s in the formula (CF), h and i indicate average degrees of polymerization, h represents 1 to 20, and i represents 0 to 20. h's and is in the two Rf₁'s may be identical to each other or may be different from each other.)
(in two Rf₁'s in the formula (CG), h and i indicate average degrees of polymerization, h represents 1 to 20, and i represents 0 to 20. h's and i's in the two Rf₁'s may be identical to each other or may be different from each other.)
(in two Rf₁'s in the formula (CH), h and i indicate average degrees of polymerization, h represents 1 to 20, and i represents 0 to 20. h's and i's in the two Rf₁'s may be identical to each other or may be different from each other.)
(in two Rf₁'s in the formula (Cl), h and i indicate average degrees of polymerization, h represents 1 to 20, and i represents 0 to 20. h's and i's in the two Rf₁'s may be identical to each other or may be different from each other.)

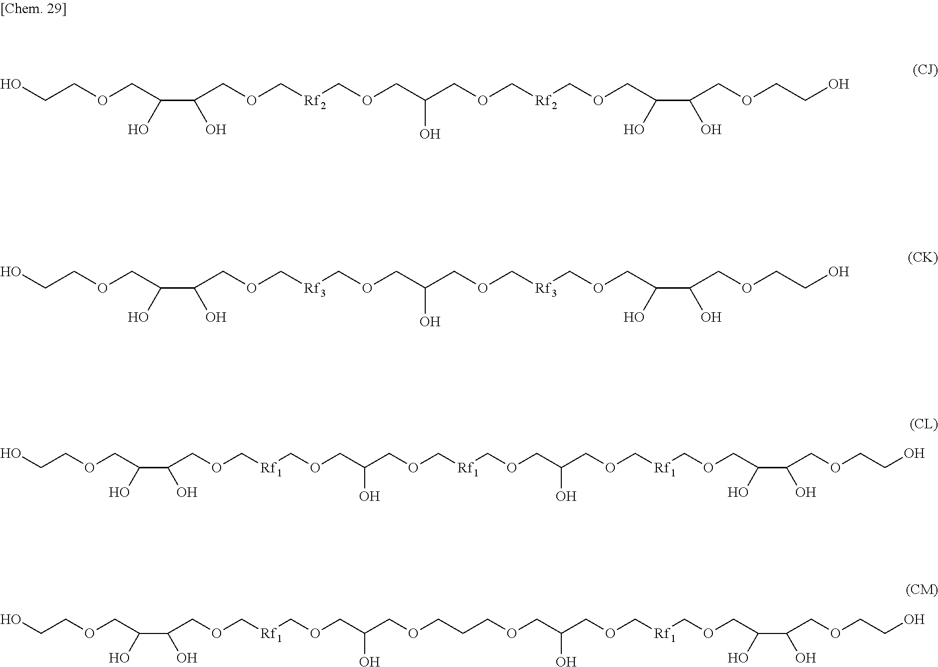

(in two Rf₂'s in the formula (CJ), j indicates an average degree of polymerization and represents 1 to 15. j's in the two Rf₂'s may be identical to each other or may be different from each other.)
(in two Rf₃'s in the formula (CK). k indicates an average degree of polymerization and represents 1 to 10. k's in the two Rf₃'s may be identical to each other or may be different from each other.)
(in three Rf₁'s in the formula (CL), h and i indicate average degrees of polymerization, h represents 1 to 20, and i represents 0 to 20. h's and i's in the three Rf₁'s may be identical to each other or may be different from each other.)
(in two Rf₁'s in the formula (CM), h and i indicate average degrees of polymerization, h represents 1 to 20, and i represents 0 to 20. h's and i's in the two Rf₁'s may be identical to each other or may be different from each other.)

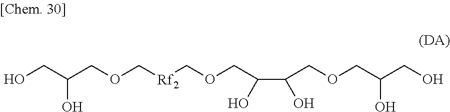

(DA)
(in Rf₂ in the formula (DA), j indicates an average degree of polymerization and represents 1 to 15.)

The number-average molecular weight (Mn) of the fluorine-containing ether compound of the present embodiment is preferably within a range of 500 to 10000 and more preferably within a range of 1000 to 5000. When the number-average molecular weight is 500 or more, a lubricating layer made of a lubricant containing the fluorine-containing ether compound of the present embodiment has excellent heat resistance. The number-average molecular weight of the fluorine-containing ether compound is more preferably 1000 or more. In addition, when the number-average molecular weight is 10000 or less, the viscosity of the fluorine-containing ether compound becomes appropriate, and a lubricating layer having a thin film thickness can be easily formed by applying a lubricant containing the fluorine-containing ether compound. The number-average molecular weight of the fluorine-containing ether compound is preferably 5000 or less since a lubricant has a viscosity that makes the lubricant easy to handle in the case of being applied to the lubricant.

The number-average molecular weight (Mn) of the fluorine-containing ether compound is a value measured by ¹H-NMR and ¹⁹F-NMR with AVANCE III 400 manufactured by Bruker Biospin Group. Specifically, the number of repeating units of the PFPE chain is calculated from an integral value measured by ¹⁹F-NMR, and the number-average molecular weight is obtained. In the NMR (nuclear magnetic resonance) measurement, a sample is diluted into a hexafluorobenzene/d-acetone (4/lv/v) solvent and measured. As 19F-NMR chemical shift standards, the peak of hexafluorobenzene is set to −164.7 ppm, and as ¹H-NMR chemical shift standards, the peak of acetone is set to 2.2 ppm.

For the fluorine-containing ether compound of the present embodiment, the molecular weight dispersity (weight-average molecular weight (Mw)/number-average molecular weight (Mn) ratio) is preferably made to be 1.3 or less by molecular weight fractionation by an appropriate method. In the present embodiment, a method for the molecular weight fractionation is not particularly limited, and it is possible to use, for example, molecular weight fractionation by silica gel column chromatography, gel permeation chromatography (GPC), or the like, molecular weight fractionation by a supercritical extraction method, and the like.

### Production Method

A method for producing the fluorine-containing ether compound of the present embodiment is not particularly limited, and the fluorine-containing ether compound can be produced by a known conventional method. The fluorine-containing ether compound of the present embodiment can be produced using, for example, a production method to be described below.

First Production Method (Case Where x is 0, and R¹ and R⁴ are Identical to Each Other)

In order to produce a compound for which x is 0 and R¹ and R⁴ are identical to each other in the formula (1), first, a fluorine-based compound having hydroxymethyl groups (—CH₂OH) disposed at both terminals of a perfluoropolyether chain corresponding to R² in the formula (1), respectively, is prepared.

Next, hydroxyl groups of the hydroxymethyl groups disposed at both terminals of the fluorine-based compound and an epoxy group of an epoxy compound having groups corresponding to R¹ (=groups corresponding to R⁴) in the formula (1) are reacted with each other. This makes it possible to obtain a compound having the groups corresponding to R¹ (=the groups corresponding to R⁴) at both terminals of the perfluoropolyether chain corresponding to R².

The epoxy compound having groups corresponding to R¹ (=groups corresponding to R⁴) in the formula (1) is an epoxy compound corresponding to the terminal group represented by the formula (2), and it is possible to use, for example, compounds represented by the following formulae (6-1) to (6-7) and (6-17) and the like. In the formulae (6-1), (6-2), (6-4) to (6-7), and (6-17), THP represents a tetrahydropyranyl group.

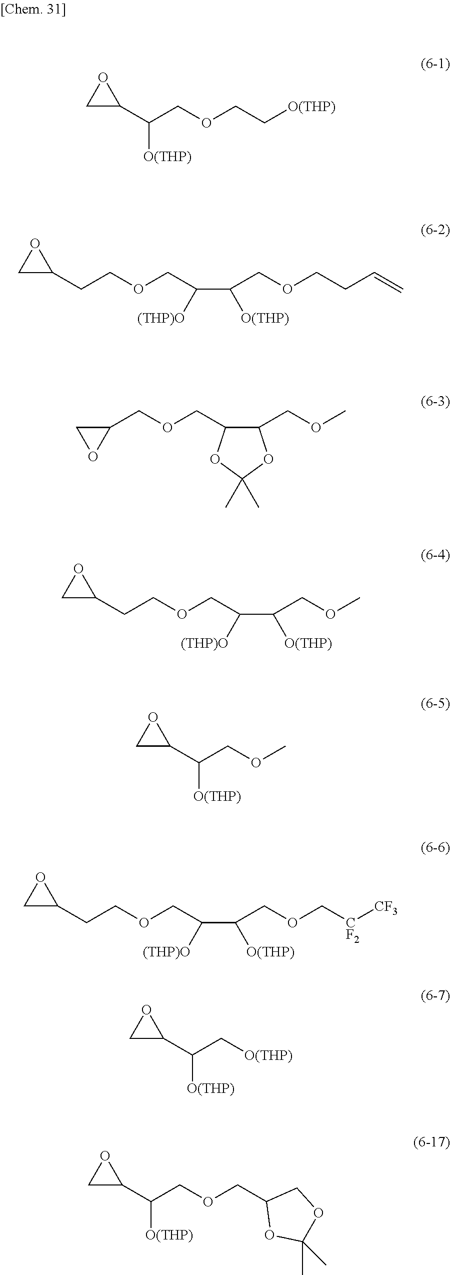

In the case of reacting the fluorine-based compound and the epoxy compound having groups corresponding to R¹ (═R⁴) in the formula (1), the epoxy compound may be reacted with the fluorine-based compound after hydroxyl groups in the epoxy compound are protected with appropriate protective groups.

The epoxy compound can be produced using, for example, a method in which an alcohol compound in which a hydroxyl group that does not get involved in the reaction may be appropriately protected and a halogen compound having an epoxy group are reacted with each other. Specifically, the epoxy compound represented by the formula (6-3) can be produced using, for example, a method in which, as shown in the following formula (7-1), an alcohol compound in which a hydroxyl group that does not get involved in the reaction may be appropriately protected and epibromohydrin indicated by the formula (8-1) are reacted with each other.

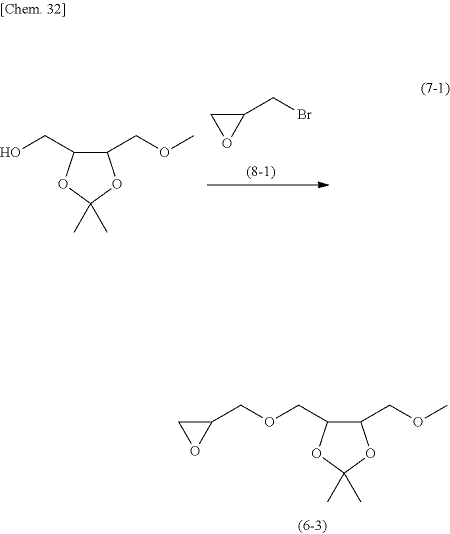

In addition, the epoxy compound may be produced using, for example, a method in which a vinyl group of a compound having the vinyl group disposed at the terminal and a hydroxyl group that may be appropriately protected is oxidized by making m-chloroperbenzoic acid (mCPBA) act thereon. Specifically, the epoxy compound represented by the formula (6-4) can be produced using a method in which, as shown in the following formula (7-2), m-chloroperbenzoic acid (mCPBA) is made to act on a vinyl group of a compound having the vinyl group disposed at the terminal and a hydroxyl group that may be appropriately protected. In the formula (7-2), THP represents a tetrahydropyranyl group.

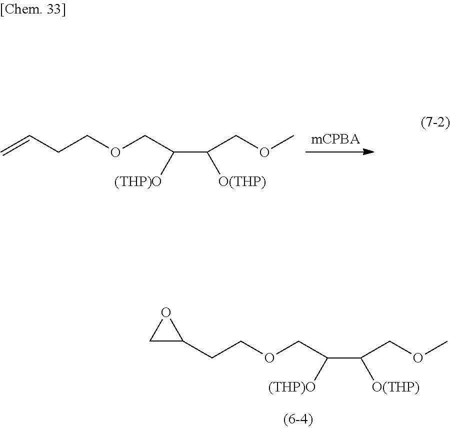

As the epoxy compound having groups corresponding to R¹ (R⁴) in the formula (1), a commercially available product may also be purchased and used.

The compound for which x is 0 and R¹ and R⁴ are identical to each other in the formula (1) can be obtained by performing the above steps.

Second Production Method (Case Where x is 0, and R¹ and R⁴ are Different from Each Other)

In order to produce a compound for which x is 0 and R¹ and R⁴ are different from each other in the formula (1), first, as in the first production method, a fluorine-based compound having hydroxymethyl groups (—CH₂OH) disposed at both terminals of a perfluoropolyether chain corresponding to R² in the formula (1), respectively, is prepared.

Next, a hydroxyl group of the hydroxymethyl group disposed at one terminal of the fluorine-based compound and an epoxy group of an epoxy compound having a group corresponding to Ri in the formula (1) are reacted with each other. This makes it possible to obtain an intermediate compound 1 having the group corresponding to R¹ at one terminal of the perfluoropolyether chain corresponding to R² (first reaction).

Next, the intermediate compound 1 and an epoxy compound having a group corresponding to R⁴ in the formula (1) are reacted with each other (second reaction).

In a case where R¹ and R⁴ are different from each other, one is the terminal group represented by the formula (2), and the other is a terminal group that does not correspond to the formula (2), as an epoxy compound corresponding to the terminal group represented by the formula (2), it is possible to use the above-described compounds represented by the formulae (6-1) to (6-7) and (6-17) and the like. In addition, as an epoxy compound corresponding to the terminal group that does not correspond to the formula (2), it is possible to use, for example, epoxy compounds represented by the following formulae (6-8) to (6-16) and the like. In the formulae (6-8), (6-10), (6-14), and (6-15), THP represents a tetrahydropyranyl group.

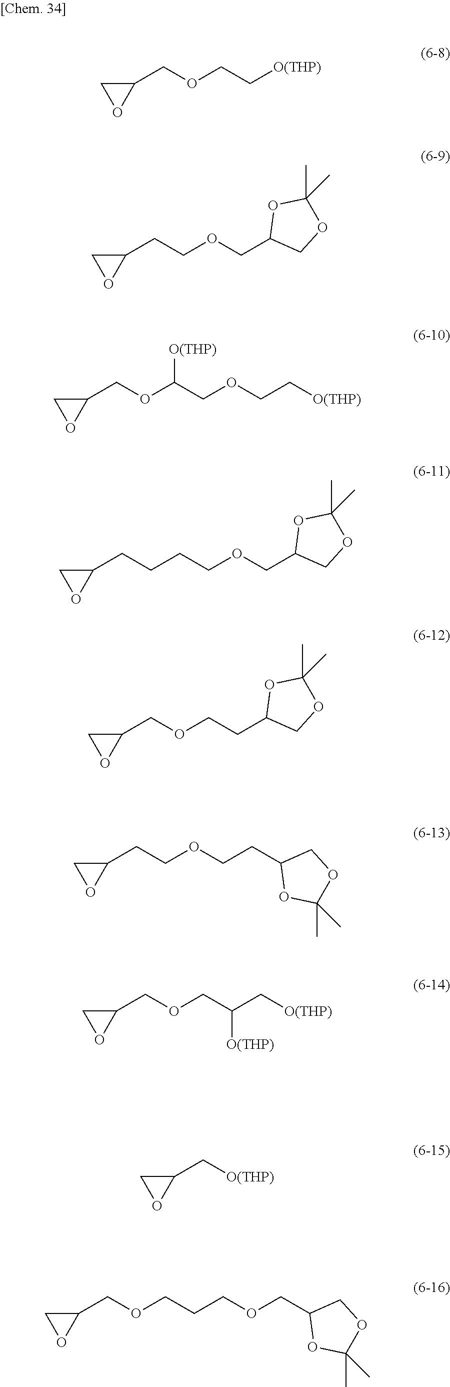

The compound for which x is 0 and R¹ and R⁴ are different from each other in the formula (1) can be obtained by performing the above steps.

Third Production Method (Case Where x is 1, R¹ and R⁴ are Identical to Each Other, and Two R²'s are Identical to Each Other)

A fluorine-based compound having hydroxymethyl groups (—CH₂OH) disposed at both terminals of a perfluoropolyether chain corresponding to R² in the formula (1), respectively, is prepared.

Next, a hydroxyl group of the hydroxymethyl group disposed at one terminal of the fluorine-based compound and an epoxy compound having a group corresponding to R¹ (=groups corresponding to R⁴) in the formula (1) are reacted with each other. This makes it possible to obtain an intermediate compound 1 having the group corresponding to R¹ (=groups corresponding to R⁴) at one terminal of the perfluoropolyether chain corresponding to R² (first reaction).

In the case of reacting the fluorine-based compound and the epoxy compound, the epoxy compound may be reacted with the fluorine-based compound after hydroxyl groups in the epoxy compound are protected with appropriate protective groups.

After that, the hydroxyl group of the hydroxymethyl group disposed at one terminal of the intermediate compound 1 generated in the above-described first reaction and a compound having an epoxy group at one end of the portion corresponding to R³ in the formula (1) and a halogen atom bonding to the other end or a compound having epoxy groups at both terminals of the portion corresponding to R³ in the formula (1) are reacted with each other (second reaction).

As the compound having an epoxy group at one end of the portion corresponding to R³ in the formula (1) and a halogen atom bonding to the other end, it is possible to use, for example, compounds represented by the following formulae (8-1) and (8-2) and the like.

As the compound having epoxy groups at both terminals of the portion corresponding to R³ in the formula (1), it is possible to use, for example, compounds represented by the following formulae (8-4) to (8-11) and the like. In the formula (8-7), THP represents a tetrahydropyranyl group.

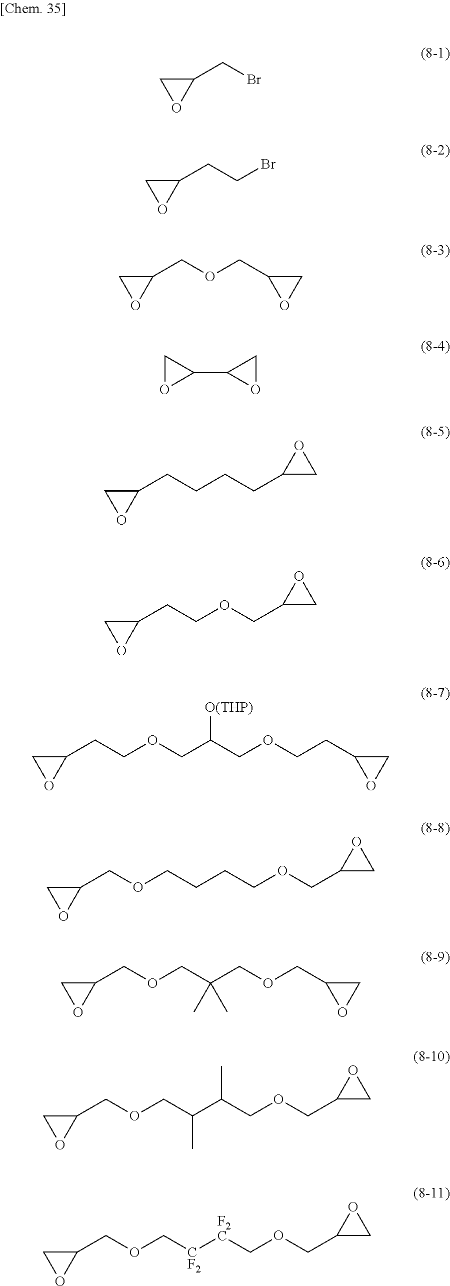

The compound having epoxy groups at both terminals can be produced using, for example, a method to be described below. That is, the compound can be produced using a method in which a diol corresponding to a part of the linking group represented by R³ and epibromohydrin twice as much as the diol in terms of the molar quantity are reacted with each other. Specifically, for example, in the case of producing the compound represented by the formula (8-8), the compound can be produced using a method in which, as shown in the following formula (9-1), 1,4-butanediol and epibromohydrin indicated by the formula (8-1) twice as much as 1,4-butanediol in terms of the molar quantity are reacted with each other.

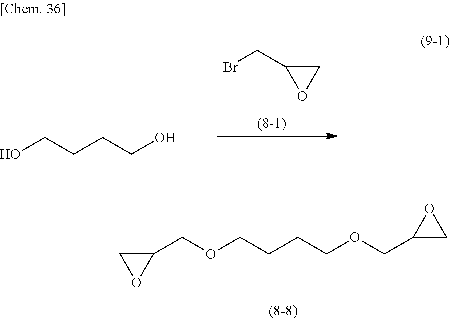 [SMILES: BrCC1CO1 ; OCCCCO ; C(CCOCC1CO1)COCC1CO1]

The compound having epoxy groups at both terminals may also be produced using a method to be described below. That is, the compound can be produced using a method in which an addition reaction is performed between a halogen compound having an epoxy group corresponding to a part of the linking group represented by R³ and an alcohol having an alkenyl group corresponding to a part of the linking group represented by R³; at this time, an alcohol having an alkenyl group twice as much as the halogen compound in terms of the molar quantity is reacted with the halogen compound, and the obtained compound is then oxidized by making m-chloroperbenzoic acid (mCPBA) act thereon. Before the compound obtained by the addition reaction is oxidized by making m-chloroperbenzoic acid (mCPBA) act thereon, a hydroxyl group generated by the addition reaction may be protected by a known method.

For example, the compound represented by the formula (8-7) can be produced using a method in which, as shown in the following formula (9-2), an addition reaction is performed between the epibromohydrin indicated by the formula (8-1) and 3-buten-1-ol twice as much as the epibromohydrin in terms of the molar quantity, and a hydroxyl group generated by the addition reaction is then protected using dihydropyran (DHP) and oxidized by making m-chloroperbenzoic acid (mCPBA) act thereon. In the formula (9-2), THP represents a tetrahydropyranyl group.

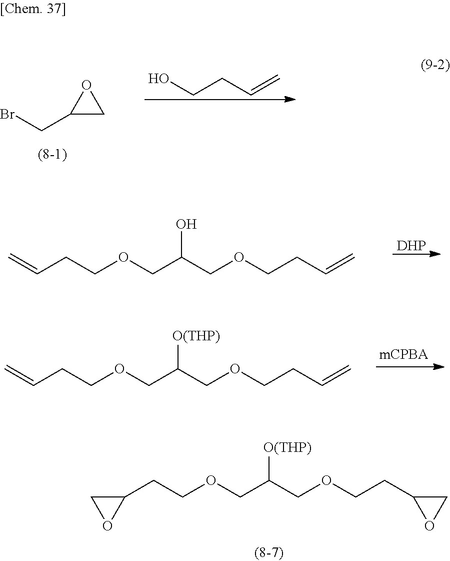

In a case where the compound obtained after the above-described second reaction has the hydroxyl group protected using a protective group, a deprotection reaction is performed using a known method.

The fluorine-containing ether compound for which x is 1, R¹ and R⁴ are identical to each other, and two R²'s are identical to each other in the formula (1) can be produced by performing the above steps.

Fourth Production Method (Case Where x is 1, R¹ and R⁴ are Different from Each Other, and Two R²'s are Different from Each Other)

First, a hydroxyl group at one terminal of a fluorine-based compound having hydroxymethyl groups disposed at both terminals of a perfluoropolyether chain corresponding to R² on the R¹ side, respectively, and an epoxy compound having a group corresponding to R¹ are reacted with each other to obtain an intermediate compound 1a (first reaction).

Next, a hydroxyl group at one terminal of a fluorine-based compound having hydroxymethyl groups disposed at both terminals of a perfluoropolyether chain corresponding to R² on the R⁴ side, respectively, and an epoxy compound having a group corresponding to R⁴ are reacted with each other to obtain an intermediate compound 1b (second reaction).

In the fourth production method, in the case of producing a fluorine-containing ether compound in which R¹ and R⁴ are identical to each other (that is, Ri and R⁴ are identical terminal groups represented by the formula (2)), as the epoxy compound having a group corresponding to R⁴, the same compound as the epoxy compound having a group corresponding to R¹, which is an epoxy compound corresponding to the terminal group represented by the formula (2), is used.

In the fourth production method, in the case of producing a fluorine-containing ether compound in which R¹ and R⁴ are different from each other and R¹ and R⁴ are both the terminal groups represented by the formula (2)), as the epoxy compound having a group corresponding to R⁴, it is possible to use, for example, an epoxy compound corresponding to the above-described terminal group represented by the formula (2) that is different from the epoxy compound having a group corresponding to R¹.

In addition, in the fourth production method, in the case of producing a fluorine-containing ether compound in which R¹ and R⁴ are different from each other, and R¹ is the terminal group represented by the formula (2), and R⁴ is a terminal group that does not correspond to the formula (2) (only one of R¹ and R⁴ is the terminal group represented by the formula (2)), as the epoxy compound having a group corresponding to R⁴, the above-described epoxy compound corresponding to the terminal group that does not correspond to the formula (2) is used.

Next, the intermediate compound 1a obtained in the first reaction and the above-described compound having an epoxy group at one end of the portion corresponding to R³ and a halogen atom bonding to the other end or a compound having epoxy groups at both terminals of the portion corresponding to R³ are reacted with each other. As a result, an intermediate compound 2a having a group corresponding to R¹ at one terminal of a perfluoropolyether chain corresponding to R² on the R¹ side and an epoxy group corresponding to R³ at the other terminal is produced (third reaction).

The intermediate compound 2a having an epoxy group may be produced by using a compound having an epoxy group at one end of the portion corresponding to R³ in the formula (1) and an alkenyl group bonding to the other end instead of the compound having an epoxy group at one end of the portion corresponding to R³ and a halogen atom bonding to the other end or the compound having epoxy groups at both terminals of the portion corresponding to R³ and oxidizing a double bond of the generated compound in the third reaction.

Subsequently, the intermediate compound 1b obtained in the second reaction and the intermediate compound 2a obtained in the third reaction are reacted with each other (fourth reaction).

In a case where the compound obtained after the above-described fourth reaction has a hydroxyl group protected using a protective group, a deprotection reaction is performed using a known method.

The fluorine-containing ether compound for which x is 1, Ri and R⁴ are different from each other, and two R²'s are different from each other in the formula (1) can be produced by performing the above steps.

Fifth Production Method (Case Where x is 2, R¹ and R⁴ are Identical to Each Other, Two R³'s are Identical to Each Other, and R² on R¹ Side and R² on R⁴ Side are Identical to Each Other)

A fluorine-based compound having hydroxymethyl groups (—CH₂OH) disposed at both terminals of a perfluoropolyether chain corresponding to R² on the R¹ side (═R² on the R⁴ side) in the formula (1), respectively, is prepared.

Next, a hydroxyl group of the hydroxymethyl group disposed at one terminal of the fluorine-based compound and an epoxy compound having a group corresponding to R¹ in the formula (1) (=a group corresponding to R⁴) are reacted with each other. This makes it possible to obtain an intermediate compound 1 having the group corresponding to R¹ (=the group corresponding to R⁴) at one terminal of the perfluoropolyether chain corresponding to R² on the R¹ side (═R² on the R⁴ side) (first reaction).

Next, a fluorine-based compound having hydroxymethyl groups (—CH₂OH) disposed at both terminals of a perfluoropolyether chain corresponding to R² in the molecular center in the formula (1) is prepared. Next, the hydroxyl groups of the hydroxymethyl groups disposed at both terminals of the fluorine-based compound and a compound having an epoxy group at one end of the above-described portion corresponding to R³ and a halogen atom bonding to the other end or a compound having epoxy groups at both terminals of the portion corresponding to R³ are reacted with each other. As a result, an intermediate compound 3a having epoxy groups corresponding to R³ at both terminals of the perfluoropolyether chain corresponding to R² in the molecular center is obtained (second reaction).

The intermediate compound 3a having epoxy groups at both terminals may be produced by using a compound having an epoxy group at one end of the portion corresponding to R³ in the formula (1) and an alkenyl group bonding to the other end instead of the compound having an epoxy group at one end of the portion corresponding to R³ and a halogen atom bonding to the other end or the compound having epoxy groups at both terminals of the portion corresponding to R³ and oxidizing a double bond of the generated compound in the second reaction.

After that, the hydroxyl group of the hydroxymethyl group disposed at one terminal of the intermediate compound 1 obtained in the first reaction and the epoxy groups disposed at both terminals of the intermediate compound 3a are reacted with each other.

In a case where the compound obtained after the above-described third reaction has a hydroxyl group protected using a protective group, a deprotection reaction is performed using a known method.

The fluorine-containing ether compound for which x is 2, R¹ and R⁴ are identical to each other, two R³'s are identical to each other, and R² on the R¹ side and R² on the R⁴ side are identical to each other in the formula (1) can be produced by performing the above steps.

Sixth Production Method (Case Where x is 2, R³ on R¹ Side and R³ on R⁴ Side are Identical to Each Other, Ri and R⁴ are Different from Each Other, and/or R² on R¹ Side and R² on R⁴ Side are Different from Each Other)

The intermediate compound 1a and the intermediate compound 1b are produced by performing the first reaction and the second reaction of the fourth production method instead of producing the intermediate compound 1 in the first reaction of the fifth production method. Next, the intermediate compound 3a is obtained in the same manner as in the second reaction of the fifth production method. In addition, the intermediate compound 1a and the intermediate compound 1b are each sequentially reacted with the epoxy groups disposed at both terminals of the intermediate compound 3a.

In a case where the compound obtained after the above-described steps has a hydroxyl group protected using a protective group, a deprotection reaction is performed using a known method.

The fluorine-containing ether compound for which x is 2, R³ on the R¹ side and R³ on the R⁴ side are identical to each other, R¹ and R⁴ are different from each other, and/or R² on the R¹ side and R² on the R⁴ side are different from each other in the formula (1) can be produced by performing the above steps.

Seventh Production Method (Case Where x is 2, R³ on Ri Side and R³ on R⁴ Side are Different from Each Other, R¹ and R⁴ are Identical to Each Other, and R² on R¹ Side and R² on R⁴ Side are Identical to Each Other)

In the second reaction of the fifth production method, a fluorine-based compound having hydroxymethyl groups (—CH₂OH) disposed at both terminals of a perfluoropolyether chain corresponding to R² in the molecular center in the formula (1) and any one compound having a portion corresponding to R³ on the Ri side selected from a compound having an epoxy group at one end of the portion corresponding to R³ and a halogen atom bonding to the other end or a compound having epoxy groups at both terminals of the portion corresponding to R³ are reacted with each other.

In addition, the obtained compound and any one compound having a portion corresponding to R³ on the R⁴ side selected from a compound having an epoxy group at one end of the portion corresponding to R³ and a halogen atom bonding to the other end or a compound having epoxy groups at both terminals of the portion corresponding to R³ are reacted with each other to obtain an intermediate compound 3b.

After that, a third reaction is performed in the same manner as in the fifth production method except that the intermediate compound 3b is used instead of the intermediate compound 3a.

In a case where the compound obtained after the above-described third reaction has a hydroxyl group protected using a protective group, a deprotection reaction is performed using a known method.

The fluorine-containing ether compound for which x is 2, R³ on the R¹ side and R³ on the R⁴ side are different from each other, R¹ and R⁴ are identical to each other, and R² on the R¹ side and R² on the R⁴ side are identical to each other in the formula (1) can be produced by performing the above steps.

A fluorine-containing ether compound for which x in the formula (1) is 2 and the perfluoropolyether chain corresponding to R² in the molecular center is identical to R² on the R¹ side and R² on the R⁴ side and different fluorine-containing ether compounds can be produced by appropriately selecting the kind of the fluorine-based compound having the perfluoropolyether chain corresponding to R² in the molecular center that is used in the fifth production method to the seventh production method.

Therefore, the fluorine-based compound having the perfluoropolyether chain corresponding to R² in the molecular center that is used in the fifth production method to the seventh production method for producing the fluorine-containing ether compound, for which x in the formula (1) is 2, may be identical to or different from a fluorine-based compound having a perfluoropolyether chain corresponding to different R².

### Lubricant for Magnetic Recording Medium

A lubricant for a magnetic recording medium of the present embodiment contains the fluorine-containing ether compound represented by the formula (1).

The lubricant of the present embodiment can be used after being mixed with a known material that is used as a material for lubricants as necessary as long as characteristics attributed to the fluorine-containing ether compound represented by the formula (1) are not impaired.

Specific examples of the known material include FOMBLIN (registered trademark) ZDIAC, FOMBLTN ZDEAL, and FOMBLIN AM-2001 (all manufactured by Solvay Solexis, Inc.), Moresco A20H (manufactured by Moresco Corporation), and the like. For the known material that is used after being mixed with the lubricant of the present embodiment, the number-average molecular weight is preferably 1000 to 10000.

In a case where the lubricant of the present embodiment contains a material other than the fluorine-containing ether compound represented by the formula (1), the content of the fluorine-containing ether compound represented by the formula (1) in the lubricant of the present embodiment is preferably 70 mass % or more, more preferably 90 mass % or more, and still more preferably 95 mass % or more.

The lubricant of the present embodiment contains the fluorine-containing ether compound represented by the formula (1) and is thus capable of forming a lubricating layer having excellent chemical substance resistance and being capable of curbing spinoff.

### Magnetic Recording Medium

A magnetic recording medium of the present embodiment has at least a magnetic layer, a protective layer, and a lubricating layer provided in order on a substrate.

The magnetic recording medium of the present embodiment can be provided with one or more base layers between the substrate and the magnetic layer as necessary. In addition, at least one of an adhesive layer and a soft magnetic layer can also be provided between the base layer and the substrate.

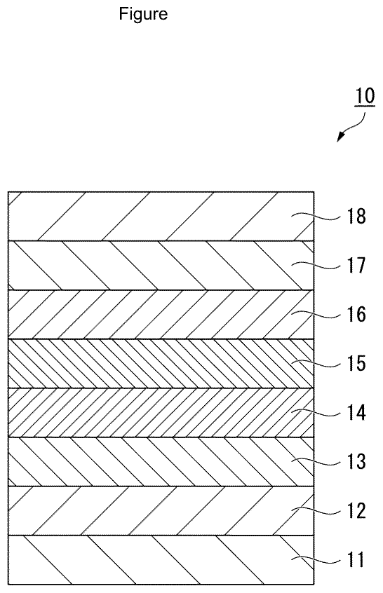

FIG. 1 is a schematic cross-sectional view showing one embodiment of a magnetic recording medium of the present invention.

A magnetic recording medium 10 of the present embodiment forms a structure in which an adhesive layer 12, a soft magnetic layer 13, a first base layer 14, a second base layer 15, a magnetic layer 16, a protective layer 17, and a lubricating layer 18 are provided in order on a substrate 11.

### Substrate

As the substrate 11, it is possible to use, for example, a non-magnetic substrate having a film made of NiP or a NiP alloy formed on a base body made of a metal or alloy material, such as Al or an Al alloy or the like.

In addition, as the substrate 11, a non-magnetic substrate made of a non-metal material, such as glass, ceramic, silicon, silicon carbide, carbon, or a resin, may be used or a non-magnetic substrate having a NiP or NiP alloy film formed on a base body made of the above-described non-metal material.

### Adhesive Layer

The adhesive layer 12 prevents the progress of corrosion of the substrate 11, which is caused in a case where the substrate 11 and the soft magnetic layer 13 provided on the adhesive layer 12 are disposed in contact with each other.

The material of the adhesive layer 12 can be appropriately selected from, for example, Cr, a Cr alloy, Ti, a Ti alloy, CrTi, NiAl, an AlRu alloy, and the like. The adhesive layer 12 can be formed by, for example, a sputtering method.

### Soft Magnetic Layer

The soft magnetic layer 13 preferably has a structure in which a first soft magnetic film, an intermediate layer made of a Ru film, and a second soft magnetic film are laminated in order. That is, the soft magnetic layer 13 preferably has a structure in which an intermediate layer made of a Ru film is inserted between two layers of soft magnetic films, whereby the soft magnetic films on and under the intermediate layer are antiferromagnetically coupled (AFC).

Examples of the material of the first soft magnetic film and the second soft magnetic film include a CoZrTa alloy, a CoFe alloy, and the like.

To the CoFe alloy that is used for the first soft magnetic film and the second soft magnetic film, any of Zr, Ta, and Nb is preferably added. This accelerates the amorphization of the first soft magnetic film and the second soft magnetic film. As a result, it becomes possible to improve the orientation of the first base layer (seed layer), and it becomes possible to reduce the flying height of a magnetic head.

The soft magnetic layer 13 can be formed by, for example, a sputtering method.

### First Base Layer

The first base layer 14 is a layer for controlling the orientation and crystal sizes of the second base layer 15 and the magnetic layer 16 provided thereon.

Examples of the first base layer 14 include layers made of a Cr layer, a Ta layer, a Ru layer, a CrMo alloy layer, a CoW alloy layer, a CrW alloy layer, a CrV alloy layer, a CrTi alloy layer, or the like.

The first base layer 14 can be formed by, for example, a sputtering method.

### Second Base Layer

The second base layer 15 is a layer for controlling the orientation of the magnetic layer 16 to be favorable. The second base layer 15 is preferably a layer made of Ru or a Ru alloy.

The second base layer 15 may be a single layer or may be composed of a plurality of layers. In a case where the second base layer 15 is composed of a plurality of layers, all of the layers may be made of the same material, or at least one layer may be made of a different material.

The second base layer 15 can be formed by, for example, a sputtering method.

### Magnetic Layer

The magnetic layer 16 is made of a magnetic film having an axis of easy magnetization vertically or horizontally oriented with respect to the substrate surface. The magnetic layer 16 is a layer containing Co and Pt. The magnetic layer 16 may be a layer containing an oxide, Cr, B, Cu, Ta, Zr, or the like to improve SNR characteristics.

Examples of the oxide that is contained in the magnetic layer 16 include SiO₂, SiO, Cr₂O₃, CoO, Ta₂O₃, TiO₂, and the like.

The magnetic layer 16 may be composed of a single layer or may be composed of a plurality of magnetic layers made of materials having different compositions.

For example, in a case where the magnetic layer 16 is composed of three layers of a first magnetic layer, a second magnetic layer, and a third magnetic layer in order from the bottom, the first magnetic layer is preferably a granular structure made of a material containing Co, Cr, and Pt and further containing an oxide. As the oxide that is contained in the first magnetic layer, for example, oxides of Cr, Si, Ta, Al, Ti, Mg, Co, and the like are preferably used. Among them, in particular, TiO₂, Cr₂O₃, SiO₂, and the like can be suitably used. In addition, the first magnetic layer is preferably made of a composite oxide to which two or more kinds of oxides have been added. Among them, in particular, Cr₂O₃—SiO₂, Cr₂O₃—TiO₂, SiO₂—TiO₂, and the like can be suitably used. The first magnetic layer may contain, aside from Co, Cr, Pt, and the oxide, one or more elements selected from B, Ta, Mo, Cu, Nd, W, Nb, Sm, Tb, Ru, and Re.

For the second magnetic layer, the same material as for the first magnetic layer can be used. The second magnetic layer is preferably a granular structure.

The third magnetic layer is preferably a non-granular structure made of a material containing Co, Cr, and Pt but not containing any oxides. The third magnetic layer may contain, aside from Co, Cr, and Pt, one or more elements selected from B, Ta, Mo, Cu, Nd, W, Nb, Sm, Tb, Ru, Re, and Mn.

In a case where the magnetic layer 16 is formed of a plurality of magnetic layers, it is preferable to provide a non-magnetic layer between the magnetic layers adjacent to each other. In a case where the magnetic layer 16 is composed of three layers of a first magnetic layer, a second magnetic layer, and a third magnetic layer, it is preferable to provide a non-magnetic layer between the first magnetic layer and the second magnetic layer and a non-magnetic layer between the second magnetic layer and the third magnetic layer.

For the non-magnetic layer that is provided between the magnetic layers adjacent to each other in the magnetic layer 16, it is possible to suitably use, for example, Ru, a Ru alloy, a CoCr alloy, a CoCrX1 alloy (X1 represents one or more elements selected from Pt, Ta, Zr, Re, Ru, Cu, Nb, Ni, Mn, Ge, Si, O, N, W, Mo, Ti, V, and B).

For the non-magnetic layer that is provided between the magnetic layers adjacent to each other in the magnetic layer 16, it is preferable to use an alloy material containing an oxide, a metal nitride, or a metal carbide. Specifically, as the oxide, it is possible to use, for example, SiO₂, Al₂O₃, Ta₂O₅, Cr₂O₃, MgO, Y₂O₃, TiO₂, and the like. As the metal nitride, it is possible to use, for example, AlN, Si₃N₄, TaN, CrN, and the like. As the metal carbide, it is possible to use, for example, TaC, BC, SiC, and the like.

The non-magnetic layer can be formed by, for example, a sputtering method.

In order to realize a higher recording density, the magnetic layer 16 is preferably a magnetic layer for perpendicular magnetic recording in which the axis of easy magnetization is vertically oriented with respect to the substrate surface. The magnetic layer 16 may be a magnetic layer for longitudinal magnetic recording.

The magnetic layer 16 may be formed by any known conventional method, such as a vapor deposition method, an ion beam sputtering method, or a magnetron sputtering method. The magnetic layer 16 is normally formed by a sputtering method.

### Protective Layer

The protective layer 17 protects the magnetic layer 16. The protective layer 17 may be a single layer or may be composed of a plurality of layers. As the protective layer 17, a carbon-based protective layer can be preferably used, and an amorphous carbon protective layer is particularly preferable. When the protective layer 17 is a carbon-based protective layer, the interaction with the polar group (particularly the hydroxyl group) that is contained in the fluorine-containing ether compound in the lubricating layer 18 is further enhanced, which is preferable.

The attachment force between the carbon-based protective layer and the lubricating layer 18 can be controlled by making the carbon-based protective layer with hydrogenated carbon and/or nitrided carbon and saving the hydrogen content and/or the nitrogen content in the carbon-based protective layer. The hydrogen content in the carbon-based protective layer is preferably 3 atomic % to 20 atomic % when measured by hydrogen forward scattering (HFS). In addition, the nitrogen content in the carbon-based protective layer is preferably 4 atomic % to 15 atomic % when measured by X-ray photoelectron spectroscopy (XPS).

The hydrogen and/or nitrogen that is contained in the carbon-based protective layer does not need to be uniformly contained in the entire carbon-based protective layer. The carbon-based protective layer is suitably made into, for example, a compositionally graded layer in which nitrogen is contained on the lubricating layer 18 side of the protective layer 17 and hydrogen is contained on the magnetic layer 16 side of the protective layer 17. In this case, the attachment forces between the magnetic layer 16 and the carbon-based protective layer and between the lubricating layer 18 and the carbon-based protective layer further improve.

The film thickness of the protective layer 17 is preferably 1 nm to 7 nm. When the film thickness of the protective layer 17 is 1 nm or more, performances as the protective layer 17 can be sufficiently obtained. The film thickness of the protective layer 17 is preferably 7 nm or less from the viewpoint of the thickness reduction of the protective layer 17.

As the film formation method of the protective layer 17, it is possible to use a sputtering method in which a carbon-containing target material is used, a CVD (chemical vapor deposition) method in which a hydrocarbon raw material, such as ethylene or toluene, is used, an IBD (ion beam deposition) method, and the like.

In the case of forming the carbon-based protective layer as the protective layer 17, the protective layer can be formed by, for example, a DC magnetron sputtering method. Particularly, in the case of forming the carbon-based protective layer as the protective layer 17, it is preferable to form an amorphous carbon protective layer by a plasma CVD method. The amorphous carbon protective layer formed by the plasma CVD method has a uniform surface and small roughness.

### Lubricating Layer

The lubricating layer 18 prevents contamination of the magnetic recording medium 10. In addition, the lubricating layer 18 reduces the friction force of a magnetic head of a magnetic recording and reproducing device that slides on the magnetic recording medium 10 to improve the durability of the magnetic recording medium 10.

The lubricating layer 18 is formed on and in contact with the protective layer 17 as shown in FIG. 1. The lubricating layer 18 is formed by applying the above-described lubricant for a magnetic recording medium of the embodiment on to the protective layer 17. Therefore, the lubricating layer 18 contains the above-described fluorine-containing ether compound.

In a case where the protective layer 17 disposed under the lubricating layer 18 is a carbon-based protective layer, the lubricating layer 18 is particularly bonded to the protective layer 17 with a high bonding force. As a result, even when the thickness of the lubricating layer 18 is thin, it becomes easy to obtain the magnetic recording medium 10 in which the surface of the protective layer 17 is coated with a high coating rate, and contamination of the surface of the magnetic recording medium 10 can be effectively prevented.

The average film thickness of the lubricating layer 18 is preferably 0.5 nm (5 Å) to 2.0 nm (20 Å) and more preferably 0.5 nm (5 Å) to 1.2 nm (12 Å). When the average film thickness of the lubricating layer 18 is 0.5 nm or more, the lubricating layer 18 does not become an island shape or a mesh shape and is formed in a uniform film thickness. Therefore, the surface of the protective layer 17 can be coated with the lubricating layer 18 with a high coating rate. In addition, when average film thickness of the lubricating layer 18 is made to be 2.0 nm or less, it is possible to sufficiently reduce the thickness of the lubricating layer 18, and the flying height of the magnetic head can be sufficiently decreased.

### Method for Forming Lubricating Layer

Examples of a method for forming the lubricating layer 18 include methods in which a magnetic recording medium that is in the middle of production and has the individual layers up to the protective layer 17 formed on the substrate 11 is prepared, and a solution for forming the lubricating layer is applied onto the protective layer 17 and dried.

The solution for forming the lubricating layer can be obtained by dispersing and dissolving the above-described lubricant for a magnetic recording medium of the present embodiment in a solvent as necessary and adjusting the viscosity and the concentration to be suitable for an application method.

Examples of the solvent that is used in the solution for forming the lubricating layer include fluorine-based solvents, such as VERTREL (registered trademark) XF (trade name, manufactured by Du Pont-Mitsui Fluorochemicals Company, Ltd.) and ASAHIKLIN (registered trademark) AE-3000 (trade name, manufactured by AGC Inc.), and the like.

A method for applying the solution for forming the lubricating layer is not particularly limited, and examples thereof include a spin coating method, a spraying method, a paper coating method, a dipping method, and the like.

In the case of using the dipping method, it is possible to use, for example, a method to be described below. First, the substrate 11 on which the individual layers up to the protective layer 17 have been formed is immersed in the solution for forming the lubricating layer that has been put into an immersion tank of a dip coater. Next, the substrate 11 is lifted from the immersion tank at a predetermined rate. This makes the solution for forming the lubricating layer applied onto the surface of the protective layer 17 on the substrate 11.

The use of the dipping method makes it possible to uniformly coat the surface of the protective layer 17 with the solution for forming the lubricating layer and makes it possible to form the lubricating layer 18 on the protective layer 17 in a uniform film thickness.

In the present embodiment, a thermal treatment is preferably performed on the substrate 11 on which the lubricating layer 18 has been formed. Thermal treatment performed improves the adhesion between the lubricating layer 18 and the protective layer 17 and improves the attachment force between the lubricating layer 18 and the protective layer 17.

Thermal treatment temperature is preferably 100° C. to 180° C. and more preferably 100° C. to 160° C. When thermal treatment temperature is 100° C. or higher, an effect of improving the adhesion between the lubricating layer 18 and the protective layer 17 can be sufficiently obtained. In addition, when thermal treatment temperature is set to 180° C. or lower, thermal decomposition of the lubricating layer 18 by a thermal treatment can be prevented. Thermal treatment time can be adjusted as appropriate depending on thermal treatment temperature and is preferably set to 10 minutes to 120 minutes.

In the present embodiment, in order to further improve the attachment force of the lubricating layer 18 to the protective layer 17, a treatment of irradiating the lubricating layer 18 before thermal treatment or after thermal treatment with ultraviolet rays (UV) may be performed.

The magnetic recording medium 10 of the present embodiment is provided with at least the magnetic layer 16, the protective layer 17, and the lubricating layer 18 in order on the substrate 11. In the magnetic recording medium 10 of the present embodiment, the lubricating layer 18 containing the above-described fluorine-containing ether compound is formed on and in contact with the protective layer 17. This lubricating layer 18 has favorable chemical substance resistance and is capable of curbing spinoff even when the film thickness is thin. Therefore, the magnetic recording medium 10 of the present embodiment is excellent in terms of reliability and durability. From this fact, the magnetic recording medium 10 of the present embodiment is capable of contributing to reduction in magnetic spacing and of lowering the flying heights of magnetic heads (for example, 10 nm or less) and stably operates for a long period of time even under harsh environments associated with diversification of the applications. Therefore, the magnetic recording medium 10 of the present embodiment is suitable as magnetic discs that are mounted particularly in LUL (load unload)-type magnetic disc devices.

### EXAMPLES

Hereinafter, the present invention will be more specifically described with examples and comparative examples. The present invention is not limited only to the following examples.

### Example 1

A compound represented by the formula (AA) was obtained by a method to be described below.

5 g of a compound represented by HOCH₂CF₂O(CF₂CF₂O)ₕ(CF₂O)ᵢCF₂CH₂OH (h indicating the average degree of polymerization in the formula was 5.0, and j was 0) (number-average molecular weight 750, molecular weight distribution 1.1), 4.2 g of a compound represented by the formula (6-1), and 5 mL of t-butanol were charged into a 100 mL eggplant flask in a nitrogen gas atmosphere and stirred at room temperature until the components became uniform to produce a mixture. 0.23 g of potassium tert-butoxide was added to this mixture and stirred at 70° C. for 16 hours to be reacted.

A compound represented by the formula (6-1) was synthesized by a method to be described below. First, a primary hydroxyl group of 3-butene-1,2-diol was protected using tert-butyldimethylchlorosilane (TBS-Cl), and a secondary hydroxyl group was then protected using dihydropyran (DHP). After that, a tert-butyldimethylsilyl (TBS) group was deprotected, and a primary hydroxyl group thus generated and 2-(2-bromoethoxy)tetrahydro-2H-pyran were reacted with each other. Finally, a vinyl group was oxidized using m-chloroperbenzoic acid (mCPBA).

A reaction solution obtained after the reaction was returned to room temperature, 10 g of a 10% hydrogen chloride-methanol solution (hydrogen chloride-methanol reagent (5% to 10%), manufactured by Tokyo Chemical Industry Co., Ltd.) was added thereto and stirred at room temperature for four hours. After that, the reaction solution was transferred to a separatory funnel containing 25 mL of a saturated sodium bicarbonate solution little by little and extracted twice with 50 mL of ethyl acetate. An organic layer was washed in order with 25 mL of a saline solution, 25 mL of a saturated sodium bicarbonate solution, and 25 mL of a saline solution and dehydrated with anhydrous sodium sulfate. After a desiccant was filtered, a filtrate was concentrated, and a residue was purified with silica gel column chromatography, thereby obtaining 4.28 g of a compound (AA) (Rf₁ in the formula (AA) was a PFPE chain represented by the formula (5-1). In Rf₁, h indicating the average degree of polymerization represented 5.0, and i indicating the average degree of polymerization represented 0).

¹H-NMR and ¹⁹F-NMR measurement of the obtained compound (AA) was performed, and the structure was identified from the following results.

¹H-NMR (acetone-D₆): δ [ppm]=3.40 to 3.85 (26H), 3.85 to 4.10 (4H)

¹⁹F-NMR (acetone-D₆): δ [ppm]=−80.5 (4F), −91.0 to −88.5 (20F)

### Example 2

A compound represented by the formula (AB) was obtained by a method to be described below.

The same operation as in Example 1 was performed except that a compound represented by HOCH₂CF₂CF₂O(CF₂CF₂CF₂O)ⱼCF₂CF₂CH₂OH (j indicating the average degree of polymerization in the formula was 4.0) (number-average molecular weight 950, molecular weight distribution 1.1) was used instead of the compound represented by HOCH₂CF₂O(CF₂CF₂O)ₕ(CF₂O)ᵢCF₂CH₂OH, and 4.41 g of a compound (AB) (Rf₂ in the formula (AB) was the PFPE chain represented by the formula (5-2). In Rf₂, j indicating the average degree of polymerization represented 4.0) was obtained.

¹H-NMR and ¹⁹F-NMR measurement of the obtained compound (AB) was performed, and the structure was identified from the following results.

¹H-NMR (acetone-D₆): δ [ppm]=3.40 to 3.85 (26H), 3.85 to 4.10 (4H)

¹⁹F-NMR (acetone-D₆): δ [ppm]=−84.0 to −83.0 (16F), −86.4 (4F), −124.3 (4F), −130.0 to −129.0 (8F)

### Example 3

A compound represented by the formula (AI) was obtained by a method to be described below.

The same operation as in Example 1 was performed except that a compound represented by the formula (6-2) was used instead of the compound represented by the formula (6-1), and 3.92 g of a compound (AI) (Rf₁ in the formula (AI) was a PFPE chain represented by the formula (5-1). In Rf₁, h indicating the average degree of polymerization represented 5.0, and i indicating the average degree of polymerization represented 0) was obtained.

The compound represented by the formula (6-2) was synthesized by a method to be described below. First, 1,3-butadiene diepoxide and 3-buten-1-ol twice as much as 1,3-butadiene diepoxide in terms of the molar quantity were reacted with each other. Next, two generated hydroxyl groups were protected using DHP. Finally, one of two vinyl groups was oxidized using mCPBA.

¹H-NMR and ¹⁹F-NMR measurement of the obtained compound (AI) was performed, and the structure was identified from the following results.

¹H-NMR (acetone-D₆): δ [ppm]=1.65 to 1.80 (411), 2.10 to 2.30 (4H), 3.40 to 3.85 (32H), 3.85 to 4.10 (4H), 4.80 to 5.10 (4H), 5.80 to 5.90 (2H)

¹⁹F-NMR (acetone-D₆): S [ppm]=−80.5 (4F), −91.0 to −88.5 (20F)

### Example 4

A compound represented by the formula (AJ) was obtained by a method to be described below.

The same operation as in Example 1 was performed except that the compound represented by the formula (6-2) was used instead of the compound represented by the formula (6-1), and the compound represented by HOCH₂CF₂CF₂O(CF₂CF₂CF₂O)CF₂CF₂CH₂OH (i indicating the average degree of polymerization in the formula was 4.0) (number-average molecular weight 950, molecular weight distribution 1.1) was used instead of the compound represented by HOCH₂CF₂O(CF₂CF₂O)ₕ(CF₂O)ᵢCF₂CH₂OH, and 4.21 g of a compound (AJ) (Rf₂ in the formula (AJ) was the PFPE chain represented by the formula (5-2). In Rf₂, j indicating the average degree of polymerization represented 4.0) was obtained.

¹H-NMR and ¹⁹F-NMR measurement of the obtained compound (AJ) was performed, and the structure was identified from the following results.

¹H-NMR (acetone-D₆): δ [ppm]=1.65 to 1.80 (4H), 2.10 to 2.30 (4H), 3.40 to 3.85 (32H), 3.85 to 4.10 (4H), 4.80 to 5.10 (4H), 5.80 to 5.90 (2H)

¹⁹F-NMR (acetone-D₆): δ [ppm]=−84.0 to −83.0 (16F), −86.4 (4F), −124.3 (4F), −130.0 to −129.0 (8F)

### Example 5

A compound represented by the formula (AN) was obtained by a method to be described below.

The same operation as in Example 1 was performed except that a compound represented by the formula (6-3) was used instead of the compound represented by the formula (6-1), and 3.85 g of a compound (AN) (Rf₁ in the formula (AN) was a PFPE chain represented by the formula (5-1). In Rf₁, h indicating the average degree of polymerization represented 5.0, and i indicating the average degree of polymerization represented 0) was obtained.

The compound represented by the formula (6-3) was synthesized by reacting methyl iodide and epibromohydrin in order with two hydroxyl groups of (+)-2,3-O-isopropylidene-L-threitol.

¹H-NMR and ¹⁹F-NMR measurement of the obtained compound (AN) was performed, and the structure was identified from the following results.

¹H-NMR (acetone-D₆): δ [ppm]=3.40 to 3.85 (34H), 3.85 to 4.10 (411)

¹⁹F-NMR (acetone-D₆): δ [ppm]=−80.5 (4F), −91.0 to −88.5 (20F)

### Example 6

A compound represented by the formula (AO) was obtained by a method to be described below.

The same operation as in Example 1 was performed except that the compound represented by the formula (6-3) was used instead of the compound represented by the formula (6-1), and the compound represented by HOCH₂CF₂CF₂O(CF₂CF₂CF₂O)ⱼCF₂CF₂CH₂OH (j indicating the average degree of polymerization in the formula was 4.0) (number-average molecular weight 950, molecular weight distribution 1.1) was used instead of the compound represented by HOCH₂CF₂O(CF₂CF₂O)ₕ(CF₂O)ᵢCF₂CH₂OH, and 4.05 g of a compound (AO) (Rf₂ in the formula (AO) was the PFPE chain represented by the formula (5-2). In Rf₂, j indicating the average degree of polymerization represented 4.0) was obtained.

¹H-NMR and ¹⁹F-NMR measurement of the obtained compound (AO) was performed, and the structure was identified from the following results.

¹H-NMR (acetone-D₆): δ [ppm]=3.40 to 3.85 (34H), 3.85 to 4.10 (411)

¹⁹F-NMR (acetone-D₆): δ [ppm]=−84.0 to −83.0 (16F), −86.4 (4F), −124.3 (4F), −130.0 to −129.0 (8F)

### Example 7

A compound represented by the formula (AP) was obtained by a method to be described below.

The same operation as in Example 1 was performed except that a compound represented by the formula (6-4) was used instead of the compound represented by the formula (6-1), and 4.21 g of a compound (AP) (Rf₁ in the formula (AP) was a PFPE chain represented by the formula (5-1). In Rf₁, h indicating the average degree of polymerization represented 5.0, and i indicating the average degree of polymerization represented 0) was obtained.

The compound represented by the formula (6-4) was synthesized by a method to be described below. First, one epoxy group of 1,3-butadiene diepoxide and the same molecular quantity of 3-buten-1-ol were reacted with each other. After that, the other epoxy group of 1,3-butadiene diepoxide and the same molecular quantity of methanol were reacted with each other. Next, two hydroxyl groups generated by the reactions were protected using DHP. Finally, a vinyl group was oxidized using mCPBA.

¹H-NMR and ¹⁹F-NMR measurement of the obtained compound (AP) was performed, and the structure was identified from the following results.

¹H-NMR (acetone-D₆): δ [ppm]=1.65 to 1.80 (411), 3.40 to 3.85 (34H), 3.85 to 4.10 (4H)

¹⁹F-NMR (acetone-D₆): δ [ppm]=−80.5 (4F), −91.0 to −88.5 (20F)

### Example 8

A compound represented by the formula (AQ) was obtained by a method to be described below.

The same operation as in Example 1 was performed except that a compound represented by the formula (6-5) was used instead of the compound represented by the formula (6-1), and the compound represented by HOCH₂CF₂CF₂O(CF₂CF₂CF₂O)ⱼCF₂CF₂CH₂OH (j indicating the average degree of polymerization in the formula was 4.0) (number-average molecular weight 950, molecular weight distribution 1.1) was used instead of the compound represented by HOCH₂CF₂O(CF₂CF₂O)ₕ(CF₂O)ᵢCF₂CH₂OH, and 3.76 g of a compound (AQ) (Rf₂ in the formula (AQ) was the PFPE chain represented by the formula (5-2). In Rf₂, j indicating the average degree of polymerization represented 4.0) was obtained.

The compound represented by the formula (6-5) was synthesized by a method to be described below. First, a primary hydroxyl group of 3-butene-1,2-diol was protected using TBS-Cl, and a secondary hydroxyl group was then protected using DHP. After that, a TBS group was deprotected, and a primary hydroxyl group thus generated and methyl iodide were reacted with each other. Finally, a vinyl group was oxidized using m-chloroperbenzoic acid (mCPBA).

¹H-NMR and ¹⁹F-NMR measurement of the obtained compound (AQ) was performed, and the structure was identified from the following results.

¹H-NMR (acetone-D₆): δ [ppm]=3.40 to 3.85 (22H), 3.85 to 4.10 (4H)

¹⁹F-NMR (acetone-D₆): δ [ppm]=−84.0 to −83.0 (16F), −86.4 (4F), −124.3 (4F), −130.0 to −129.0 (8F)

### Example 9

A compound represented by the formula (AS) was obtained by a method to be described below.

The same operation as in Example 1 was performed except that a compound represented by the formula (6-6) was used instead of the compound represented by the formula (6-1), and the compound represented by HOCH₂CF₂CF₂O(CF₂CF₂CF₂O)ⱼCF₂CF₂CH₂OH (indicating the average degree of polymerization in the formula was 4.0) (number-average molecular weight 950, molecular weight distribution 1.1) was used instead of the compound represented by HOCH₂CF₂O(CF₂CF₂O)ₕ(CF₂O)ᵢCF₂CH₂OH, and 4.85 g of a compound (AS) (Rf₂ in the formula (AS) was the PFPE chain represented by the formula (5-2). In Rf₂, j indicating the average degree of polymerization represented 4.0) was obtained.

The compound represented by the formula (6-6) was synthesized by a method to be described below. First, one epoxy group of 1,3-butadiene diepoxide and the same molecular quantity of 3-buten-1-ol were reacted with each other. Next, the other epoxy group of 1,3-butadiene diepoxide and the same molecular quantity of 2,2,3,3,3-pentafluoropropanol were reacted with each other. Next, two generated hydroxyl groups were protected using DHP. Finally, a vinyl group was oxidized using mCPBA.

¹H-NMR and ¹⁹F-NMR measurement of the obtained compound (AS) was performed, and the structure was identified from the following results.

¹H-NMR (acetone-D₆): δ [ppm]=1.65 to 1.80 (4H), 3.40 to 3.85 (28H), 3.85 to 4.10 (8H)

¹⁹F-NMR (acetone-D₆): δ [ppm]=−84.0 to −83.0 (22F), −86.4 (4F), −124.3 (4F), −130.0 to −129.0 (12F)

### Example 10

A compound represented by the formula (AW) was obtained by a method to be described below.

The same operation as in Example 1 was performed except that a compound represented by the formula (6-7) was used instead of the compound represented by the formula (6-1), and the compound represented by HOCH₂CF₂CF₂O(CF₂CF₂CF₂O)ⱼCF₂CF₂CH₂OH (j indicating the average degree of polymerization in the formula was 4.0) (number-average molecular weight 950, molecular weight distribution 1.1) was used instead of the compound represented by HOCH₂CF₂O(CF₂CF₂O)ₕ(CF₂O)ᵢCF₂CH₂OH, and 3.89 g of a compound (AW) (Rf₂ in the formula (AW) was the PFPE chain represented by the formula (5-2). In Rf₂, j indicating the average degree of polymerization represented 4.0) was obtained.

The compound represented by the formula (6-7) was synthesized by protecting two hydroxyl groups of 3-butene-1,2-diol using DHP and subsequently oxidizing a vinyl group using mCPBA.

¹H-NMR and ¹⁹F-NMR measurement of the obtained compound (AW) was performed, and the structure was identified from the following results.

¹H-NMR (acetone-D₆): δ [ppm]=3.40 to 3.85 (18H), 3.85 to 4.10 (4H)

¹⁹F-NMR (acetone-D₆): δ [ppm]=−84.0 to −83.0 (16F), −86.4 (4F), −124.3 (4F), −130.0 to −129.0 (8F)

### Example 11

A compound represented by the formula (BA) was obtained by a method to be described below.

### First Reaction

12 g of a compound represented by HOCH₂CF₂O(CF₂CF₂O)ₕ(CF₂O)ᵢCF₂CH₂OH (h indicating the average degree of polymerization in the formula was 5.0, and i was 0) (number-average molecular weight 750, molecular weight distribution 1.1), 5.1 g of a compound represented by the formula (6-2), and 12 mL of t-butanol were charged into a 100 mL eggplant flask in a nitrogen gas atmosphere and stirred at room temperature until the components became uniform to produce a mixture. 0.65 g of potassium tert-butoxide was added to this mixture and stirred at 70° C. for 16 hours to be reacted.

A reaction product obtained after the reaction was cooled to 25° C., transferred to a separatory funnel containing 100 mL of water, and extracted three times with 100 mL of ethyl acetate. An organic layer was washed with water and dehydrated with anhydrous sodium sulfate. After a desiccant was filtered, a filtrate was concentrated, and a residue was purified with silica gel column chromatography, thereby obtaining 7.0 g of a compound indicated by the following formula (10-1) as an intermediate compound 1.

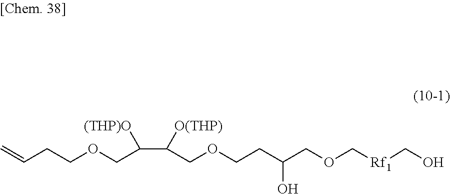

(Rf₁ in the formula (10-1) is the PFPE chain represented by the formula (5-1). In Rf₁, h indicating the average degree of polymerization represents 5.0, and i indicating the average degree of polymerization represents 0. THP represents a tetrahydropyranyl group.)

### Second Reaction

Subsequently, 7.0 g of the compound indicated by the formula (10-1), which was the intermediate compound 1 obtained above, 2.70 g of the compound represented by the formula (6-1), and 20 mL of t-butanol were charged into a 100 mL eggplant flask in a nitrogen gas atmosphere and stirred at room temperature until the components became uniform to produce a mixture. 3.3 g of potassium tert-butoxide was added to this mixture and stirred at 70° C. for 16 hours to be reacted.

A reaction solution obtained after the reaction was returned to room temperature, 50 g of a 10% hydrogen chloride-methanol solution (hydrogen chloride-methanol reagent (5% to 10%), manufactured by Tokyo Chemical Industry Co., Ltd.) was added thereto and stirred at room temperature for four hours. After that, the reaction solution was transferred to a separatory funnel containing 100 mL of a saturated sodium bicarbonate solution little by little and extracted twice with 200 mL of ethyl acetate. An organic layer was washed in order with 100 mL of a saline solution, 100 mL of a saturated sodium bicarbonate solution, and 100 mL of a saline solution and dehydrated with anhydrous sodium sulfate. After a desiccant was filtered, a filtrate was concentrated, and a residue was purified with silica gel column chromatography, thereby obtaining 4.29 g of a compound (BA) (Rf₁ in the formula (BA) is the PFPE chain represented by the formula (5-1). In Rf₁, h indicating the average degree of polymerization represented 5.0, and i indicating the average degree of polymerization represented 0).

¹H-NMR and ¹⁹F-NMR measurement of the obtained compound (BA) was performed, and the structure was identified from the following results.

¹H-NMR (acetone-D₆): δ [ppm]=1.65 to 1.80 (2H), 2.10 to 2.30 (2H), 3.40 to 3.85 (29H), 3.85 to 4.10 (4H), 4.80 to 5.10 (2H), 5.80 to 5.90 (11H)

¹⁹F-NMR (acetone-D₆): δ [ppm]=−80.5 (4F), −91.0 to −88.5 (20F)

### Example 12

A compound represented by the formula (BB) was obtained by a method to be described below.

The same operation as in Example 11 was performed except that the compound represented by HOCH₂CF₂CF₂O(CF₂CF₂CF₂O)ⱼCF₂CF₂CH₂OH (j indicating the average degree of polymerization in the formula was 4.0) (number-average molecular weight 950, molecular weight distribution 1.1) was used instead of the compound represented by HOCH₂CF₂O(CF₂CF₂O)ₕ(CF₂O)ᵢCF₂CH₂OH, and 4.48 g of a compound (BB) (Rf₂ in the formula (BB) was the PFPE chain represented by the formula (5-2). In Rf₂, j indicating the average degree of polymerization represented 4.0) was obtained.

¹H-NMR and ¹⁹F-NMR measurement of the obtained compound (BB) was performed, and the structure was identified from the following results.

¹H-NMR (acetone-D₆): δ [ppm]=1.65 to 1.80 (2H), 2.10 to 2.30 (2H), 3.40 to 3.85 (29H), 3.85 to 4.10 (4H), 4.80 to 5.10 (2H), 5.80 to 5.90 (1H)

¹⁹F-NMR (acetone-D₆): δ [ppm]=−84.0 to −83.0 (16F), −86.4 (4F), −124.3 (4F), −130.0 to −129.0 (8F)

### Example 13

A compound represented by the formula (BC) was obtained by a method to be described below.

The same operation as in Example 11 was performed except that the compound represented by the formula (6-4) was used instead of the compound represented by the formula (6-1), and the compound represented by HOCH₂CF₂CF₂O(CF₂CF₂CF₂O)ⱼCF₂CF₂CH₂OH (indicating the average degree of polymerization in the formula was 4.0) (number-average molecular weight 950, molecular weight distribution 1.1) was used instead of the compound represented by HOCH₂CF₂O(CF₂CF₂O)ₕ(CF₂O)ᵢCF₂CH₂OH, and 4.15 g of a compound (BC) (Rf₂ in the formula (BC) was the PFPE chain represented by the formula (5-2). In Rf₂, j indicating the average degree of polymerization represented 4.0) was obtained.

¹H-NMR and ¹⁹F-NMR measurement of the obtained compound (BC) was performed, and the structure was identified from the following results.

¹H-NMR (acetone-D₆): δ [ppm]=1.65 to 1.80 (2H), 2.10 to 2.30 (211), 3.40 to 3.85 (31H), 3.85 to 4.10 (4H), 4.80 to 5.10 (2H), 5.80 to 5.90 (1H)

¹⁹F-NMR (acetone-D₆): δ [ppm]=−84.0 to −83.0 (16F), −86.4 (4F), −124.3 (4F), −130.0 to −129.0 (8F)

### Example 14

A compound represented by the formula (BD) was obtained by a method to be described below.

The same operation as in Example 11 was performed except that a compound represented by the formula (6-8) was used instead of the compound represented by the formula (6-2), and the compound represented by HOCH₂CF₂CF₂O(CF₂CF₂CF₂O)ⱼCF₂CF₂CH₂OH (j indicating the average degree of polymerization in the formula was 4.0) (number-average molecular weight 950, molecular weight distribution 1.1) was used instead of the compound represented by HOCH₂CF₂O(CF₂CF₂O)ₕ(CF₂O)ᵢCF₂CH₂OH, and 3.98 g of a compound (BD) (Rf₂ in the formula (BD) was the PFPE chain represented by the formula (5-2). In Rf₂, j indicating the average degree of polymerization represented 4.0) was obtained.

The compound represented by the formula (6-8) was synthesized by protecting a hydroxyl group of ethylene glycol monoallyl ether using DHP and then oxidizing a vinyl group using mCPBA.

¹H-NMR and ¹⁹F-NMR measurement of the obtained compound (BD) was performed, and the structure was identified from the following results.

¹H-NMR (acetone-D₆): δ [ppm]=3.40 to 3.85 (24H), 3.85 to 4.10 (4H)

¹⁹F-NMR (acetone-D₆): δ [ppm]=−84.0 to −83.0 (16F), −86.4 (4F), −124.3 (4F), −130.0 to −129.0 (8F)

### Example 15

A compound represented by the formula (BE) was obtained by a method to be described below.

The same operation as in Example 11 was performed except that a compound represented by the formula (6-9) was used instead of the compound represented by the formula (6-2), and 4.22 g of a compound (BE) (Rf₁ in the formula (BE) was the PFPE chain represented by the formula (5-1). In Rf₁, h indicating the average degree of polymerization represented 5.0, and i indicating the average degree of polymerization represented 0) was obtained.

The compound represented by the formula (6-9) was synthesized by reacting a compound obtained by brominating a hydroxyl group of solketal (2,2-dimethyl-1,3-dioxolane-4-methanol) and 3-buten-1-ol, and oxidizing a vinyl group of the obtained compound using mCPBA.

¹H-NMR and ¹⁹F-NMR measurement of the obtained compound (BE) was performed, and the structure was identified from the following results.

¹H-NMR (acetone-D₆): δ [ppm]=1.65 to 1.85 (2H), 3.40 to 3.85 (29H), 3.85 to 4.10 (4H)

¹⁹F-NMR (acetone-D₆): δ [ppm]=−80.5 (4F), −91.0 to −88.5 (20F)

### Example 16

A compound represented by the formula (BF) was obtained by a method to be described below.

The same operation as in Example 11 was performed except that a compound represented by the formula (6-10) was used instead of the compound represented by the formula (6-1), and the compound represented by HOCH₂CF₂CF₂O(CF₂CF₂CF₂O)ⱼCF₂CF₂CH₂OH (j indicating the average degree of polymerization in the formula was 4.0) (number-average molecular weight 950, molecular weight distribution 1.1) was used instead of the compound represented by HOCH₂CF₂O(CF₂CF₂O)ₕ(CF₂O)ᵢCF₂CH₂OH, and 4.17 g of a compound (BF) (Rf₂ in the formula (BF) was the PFPE chain represented by the formula (5-2). In Rf₂, j indicating the average degree of polymerization represented 4.0) was obtained.

The compound represented by the formula (6-10) was synthesized by reacting 3-buten-1-ol and the compound represented by the formula (6-8), protecting the generated hydroxyl group using DHP, and then oxidizing a vinyl group using mCPBA.

¹H-NMR and ¹⁹F-NMR measurement of the obtained compound (BF) was performed, and the structure was identified from the following results.

¹H-NMR (acetone-D₆): δ [ppm]=1.65 to 1.80 (4H), 2.10 to 2.30 (211), 3.40 to 3.85 (33H), 3.85 to 4.10 (411), 4.80 to 5.10 (211), 5.80 to 5.90 (1H)

¹⁹F-NMR (acetone-D₆): δ [ppm]=−84.0 to −83.0 (16F), −86.4 (4F), −124.3 (4F), −130.0 to −129.0 (8F)

### Example 17

A compound represented by the formula (BG) was obtained by a method to be described below.

The same operation as in Example 11 was performed except that the compound represented by the formula (6-9) was used instead of the compound represented by the formula (6-1), and the compound represented by HOCH₂CF₂CF₂O(CF₂CF₂CF₂O)ⱼCF₂CF₂CH₂OH (j indicating the average degree of polymerization in the formula was 4.0) (number-average molecular weight 950, molecular weight distribution 1.1) was used instead of the compound represented by HOCH₂CF₂O(CF₂CF₂O)ₕ(CF₂O)ᵢCF₂CH₂OH, and 4.52 g of a compound (BG) (Rf₂ in the formula (BG) was the PFPE chain represented by the formula (5-2). In Rf₂, j indicating the average degree of polymerization represented 4.0) was obtained.

¹H-NMR and ¹⁹F-NMR measurement of the obtained compound (BG) was performed, and the structure was identified from the following results.

¹H-NMR (acetone-D₆): δ [ppm]=1.65 to 1.80 (4H), 2.10 to 2.30 (2H), 3.40 to 3.85 (29H), 3.85 to 4.10 (4H), 4.80 to 5.10 (2H), 5.80 to 5.90 (1H)

¹⁹F-NMR (acetone-D₆): δ [ppm]=−84.0 to −83.0 (16F), −86.4 (4F), −124.3 (4F), −130.0 to −129.0 (8F)

### Example 18

A compound represented by the formula (BH) was obtained by a method to be described below.

The same operation as in Example 11 was performed except that a compound represented by the formula (6-11) was used instead of the compound represented by the formula (6-1), and the compound represented by HOCH₂CF₂CF₂O(CF₂CF₂CF₂O)ⱼCF₂CF₂CH₂OH (j indicating the average degree of polymerization in the formula was 4.0) (number-average molecular weight 950, molecular weight distribution 1.1) was used instead of the compound represented by HOCH₂CF₂O(CF₂CF₂O)ₕ(CF₂O)ᵢCF₂CH₂OH, and 4.34 g of a compound (BH) (Rf₂ in the formula (BH) was the PFPE chain represented by the formula (5-2). In Rf₂, j indicating the average degree of polymerization represented 4.0) was obtained.

The compound represented by the formula (6-11) was synthesized by reacting a compound obtained by brominating a hydroxyl group of solketal and 5-hexen-1-ol, and oxidizing a vinyl group of the obtained compound using mCPBA.

¹H-NMR and ¹⁹F-NMR measurement of the obtained compound (BH) was performed, and the structure was identified from the following results.

¹H-NMR (acetone-D₆): δ [ppm]=1.45 to 1.85 (8H), 2.10 to 2.30 (211), 3.40 to 3.85 (29H), 3.85 to 4.10 (411), 4.80 to 5.10 (211), 5.80 to 5.90 (111)

¹⁹F-NMR (acetone-D₆): δ [ppm]=−84.0 to −83.0 (16F), −86.4 (4F), −124.3 (4F), −130.0 to −129.0 (8F)

### Example 19

A compound represented by the formula (BI) was obtained by a method to be described below.

The same operation as in Example 11 was performed except that a compound represented by the formula (6-12) was used instead of the compound represented by the formula (6-1), and the compound represented by HOCH₂CF₂CF₂O(CF₂CF₂CF₂O)ⱼCF₂CF₂CH₂OH (j indicating the average degree of polymerization in the formula was 4.0) (number-average molecular weight 950, molecular weight distribution 1.1) was used instead of the compound represented by HOCH₂CF₂O(CF₂CF₂O)ₕ(CF₂O)ᵢCF₂CH₂OH, and 4.61 g of a compound (BI) (Rf₂ in the formula (BI) was the PFPE chain represented by the formula (5-2). In Rf₂, j indicating the average degree of polymerization represented 4.0) was obtained.

The compound represented by the formula (6-12) was synthesized by protecting the 1,2-diol portion of 1,2,4-butanetriol with acetone and then reacting a hydroxyl group at the position 4 and epibromohydrin.

¹H-NMR and ¹⁹F-NMR measurement of the obtained compound (BI) was performed, and the structure was identified from the following results.

¹H-NMR (acetone-D₆): δ [ppm]=1.45 to 1.85 (8H), 2.10 to 2.30 (2H), 3.40 to 3.85 (29H), 3.85 to 4.10 (4H), 4.80 to 5.10 (2H), 5.80 to 5.90 (1H)

¹⁹F-NMR (acetone-D₆): δ [ppm]=−84.0 to −83.0 (16F), −86.4 (4F), −124.3 (4F), −130.0 to −129.0 (8F)

### Example 20

A compound represented by the formula (BJ) was obtained by a method to be described below.

The same operation as in Example 11 was performed except that a compound represented by the formula (6-13) was used instead of the compound represented by the formula (6-1), and the compound represented by HOCH₂CF₂CF₂O(CF₂CF₂CF₂O)ⱼCF₂CF₂CH₂OH (j indicating the average degree of polymerization in the formula was 4.0) (number-average molecular weight 950, molecular weight distribution 1.1) was used instead of the compound represented by HOCH₂CF₂O(CF₂CF₂O)ₕ(CF₂₀)ᵢCF₂CH₂OH, and 4.42 g of a compound (BJ) (Rf₂ in the formula (BJ) was the PFPE chain represented by the formula (5-2). In Rf₂, j indicating the average degree of polymerization represented 4.0) was obtained.

The compound represented by the formula (6-13) was synthesized by a method to be described below. The 1,2-diol portion of 1,2,4-butanetriol was protected using acetone, and a hydroxyl group at the position 4 was then brominated and reacted with 3-buten-1-ol to obtain a compound. A vinyl group of the obtained compound was oxidized using mCPBA, thereby synthesizing the compound.

¹H-NMR and ¹⁹F-NMR measurement of the obtained compound (BJ) was performed, and the structure was identified from the following results.

¹H-NMR (acetone-D₆): δ [ppm]=1.65 to 1.80 (6H), 2.10 to 2.30 (211), 3.40 to 3.85 (29H), 3.85 to 4.10 (4H), 4.80 to 5.10 (211), 5.80 to 5.90 (1H)

¹⁹F-NMR (acetone-D₆): δ [ppm]=−84.0 to −83.0 (16F), −86.4 (4F), −124.3 (4F), −130.0 to −129.0 (8F)

### Example 21

A compound represented by the formula (BK) was obtained by a method to be described below.

The same operation as in Example 11 was performed except that a compound represented by the formula (6-14) was used instead of the compound represented by the formula (6-1), and the compound represented by HOCH₂CF₂CF₂O(CF₂CF₂CF₂O)ⱼCF₂CF₂CH₂OH (j indicating the average degree of polymerization in the formula was 4.0) (number-average molecular weight 950, molecular weight distribution 1.1) was used instead of the compound represented by HOCH₂CF₂O(CF₂CF₂O)ₕ(CF₂O)ᵢCF₂CH₂OH, and 4.37 g of a compound (BK) (Rf₂ in the formula (BK) was the PFPE chain represented by the formula (5-2). In Rf₂, j indicating the average degree of polymerization represented 4.0) was obtained.

The compound represented by the formula (6-14) was synthesized by protecting two hydroxyl groups of 3-allyloxy-1,2-propanediol using dihydropyran and then oxidizing a vinyl group with mCPBA.

¹H-NMR and ¹⁹F-NMR measurement of the obtained compound (BK) was performed, and the structure was identified from the following results.

¹H-NMR (acetone-D₆): δ [ppm]=1.65 to 1.80 (2H), 2.10 to 2.30 (2H), 3.40 to 3.85 (29H), 3.85 to 4.10 (4H), 4.80 to 5.10 (2H), 5.80 to 5.90 (1H)

¹⁹F-NMR (acetone-D₆): δ [ppm]=−84.0 to −83.0 (16F), −86.4 (4F), −124.3 (4F), −130.0 to −129.0 (8F)

### Example 22

A compound represented by the formula (BL) was obtained by a method to be described below.

The same operation as in Example 11 was performed except that a compound represented by the formula (6-15) was used instead of the compound represented by the formula (6-1), and the compound represented by HOCH₂CF₂CF₂O(CF₂CF₂CF₂O)ⱼCF₂CF₂CH₂OH (j indicating the average degree of polymerization in the formula was 4.0) (number-average molecular weight 950, molecular weight distribution 1.1) was used instead of the compound represented by HOCH₂CF₂O(CF₂CF₂O)ₕ(CF₂O)ᵢCF₂CH₂OH, and 3.63 g of a compound (BL) (Rf₂ in the formula (BL) was the PFPE chain represented by the formula (5-2). In Rf₂, j indicating the average degree of polymerization represented 4.0) was obtained.

The compound represented by the formula (6-15) was synthesized by oxidizing a vinyl group of 2-(allyloxy)tetrahydropyran with mCPBA.

¹H-NMR and ¹⁹F-NMR measurement of the obtained compound (BL) was performed, and the structure was identified from the following results.

¹H-NMR (acetone-D₆): δ [ppm]=1.65 to 1.80 (2H), 2.10 to 2.30 (2H), 3.40 to 3.85 (23H), 3.85 to 4.10 (4H), 4.80 to 5.10 (211), 5.80 to 5.90 (1H)

¹⁹F-NMR (acetone-D₆): δ [ppm]=−84.0 to −83.0 (16F), −86.4 (4F), −124.3 (4F), −130.0 to −129.0 (8F)

### Example 23

A compound represented by the formula (BM) was obtained by a method to be described below.

The same operation as in Example 11 was performed except that a compound represented by the formula (6-16) was used instead of the compound represented by the formula (6-1), and the compound represented by HOCH₂CF₂CF₂O(CF₂CF₂CF₂O)ⱼCF₂CF₂CH₂OH (j indicating the average degree of polymerization in the formula was 4.0) (number-average molecular weight 950, molecular weight distribution 1.1) was used instead of the compound represented by HOCH₂CF₂O(CF₂CF₂O)ₕ(CF₂O)ᵢCF₂CH₂OH, and 4.35 g of a compound (BM) (Rf₂ in the formula (BM) was the PFPE chain represented by the formula (5-2). In Rf₂, j indicating the average degree of polymerization represented 4.0) was obtained.

The compound represented by the formula (6-16) was synthesized by reacting one hydroxyl group of 1,3-propanediol and a compound obtained by brominating a hydroxyl group of solketal and then reacting the other hydroxyl group of 1,3-propanediol and epibromohydrin.

¹H-NMR and ¹⁹F-NMR measurement of the obtained compound (BM) was performed, and the structure was identified from the following results.

¹H-NMR (acetone-D₆): δ [ppm]=1.65 to 1.80 (4H), 2.10 to 2.30 (2H), 3.40 to 3.85 (33H), 3.85 to 4.10 (4H), 4.80 to 5.10 (2H), 5.80 to 5.90 (1H)

¹⁹F-NMR (acetone-D₆): δ [ppm]=−84.0 to −83.0 (16F), −86.4 (4F), −124.3 (4F), −130.0 to −129.0 (8F)

### Example 24

A compound represented by the formula (CA) was obtained by a method to be described below.

First, the same operation as the first reaction in Example 11 was performed except that the compound represented by the formula (6-1) was used instead of the compound represented by the formula (6-2), and 6.7 g of a compound indicated by the following formula (10-2) was obtained as an intermediate compound 1.

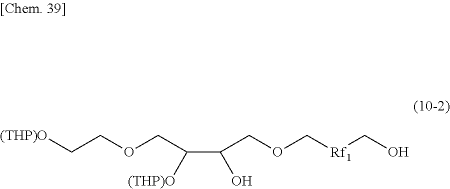

(Rf₁ in the formula (10-2) is the PFPE chain represented by the formula (5-1). In Rf₁, h indicating the average degree of polymerization represents 5.0, and i indicating the average degree of polymerization represents 0. THP represents a tetrahydropyranyl group.)

Next, 6.7 g of the compound indicated by the formula (10-2), which was the intermediate compound 1 obtained above, 0.55 g of epibromohydrin indicated by the formula (8-1), and 10 mL of t-butanol were charged into a 100 mL eggplant flask in a nitrogen gas atmosphere and stirred until the components became uniform at room temperature. 0.72 g of potassium tert-butoxide was added to this uniform liquid and stirred at 70° C. for 23 hours to be reacted.

A reaction solution obtained after the reaction was returned to room temperature, 10 g of a 10% hydrogen chloride-methanol solution (hydrogen chloride-methanol reagent (5% to 10%), manufactured by Tokyo Chemical Industry Co., Ltd.) was added thereto and stirred at room temperature for four hours. After that, the reaction solution was transferred to a separatory funnel containing 25 mL of a saturated sodium bicarbonate solution little by little and extracted twice with 50 mL of ethyl acetate. An organic layer was washed in order with 25 mL of a saline solution, 25 mL of a saturated sodium bicarbonate solution, and 25 mL of a saline solution and dehydrated with anhydrous sodium sulfate. After a desiccant was filtered, a filtrate was concentrated, and a residue was purified with silica gel column chromatography, thereby obtaining 3.41 g of a compound (CA) (two Rf₁'s in the formula (CA) is the PFPE chain represented by the formula (5-1). In the two Rf₁'s, h indicating the average degree of polymerization represented 5.0, and i indicating the average degree of polymerization represented 0).

¹H-NMR and ¹⁹F-NMR measurement of the obtained compound (CA) was performed, and the structure was identified from the following results.

¹H-NMR (acetone-D₆): δ [ppm]=3.40 to 3.85 (32H), 3.85 to 4.10 (8H)

¹⁹F-NMR (acetone-D₆): δ [ppm]=−80.5 (8F), −91.0 to −88.5 (40F)

The structures of R¹, R², R³, and R⁴ at the time of applying the compounds of Examples 1 to 24 obtained as described above to the formula (1), respectively, are shown in Table 1.

### Example 25

A compound represented by the formula (DA) was obtained by a method to be described below.

The same operation as in Example 22 was performed except that a compound represented by the formula (6-17) was used instead of the compound represented by the formula (6-2), and 3.12 g of a compound (DA) (Rf₂ in the formula (DA) was the PFPE chain represented by the formula (5-2). In Rf₂, j indicating the average degree of polymerization represented 4.0) was obtained.

The compound represented by the formula (6-17) was synthesized by a method to be described below. First, p-toluenesulfonyl chloride was made to act on a hydroxyl group of solketal, thereby obtaining a compound in which the hydroxyl group of solketal was tosylated. Subsequently, a primary hydroxyl group of 3-butene-1,2-diol was protected using tert-butyldimethylchlorosilane (TBS-Cl), and a secondary hydroxyl group was then protected using dihydropyran (DHP). After that, a primary hydroxyl group generated by deprotecting a tert-butyldimethylsilyl (TBS) group and the compound in which the hydroxyl group of solketal was tosylated were reacted with each other. Finally, a vinyl group was oxidized using m-chloroperbenzoic acid (mCPBA).

¹H-NMR and ¹⁹F-NMR measurement of the obtained compound (DA) was performed, and the structure was identified from the following results.

¹H-NMR (acetone-D₆): δ [ppm]=3.40 to 3.85 (22H), 3.85 to 4.10 (411)

¹⁹F-NMR (acetone-D₆): δ [ppm]=−84.0 to −83.0 (16F), −86.4 (4F), −124.3 (4F), −130.0 to −129.0 (8F)

TABLE 1

| | Compound | x | R¹ | | R² | R³ | | R⁴ | |
|---|---|---|---|---|---|---|---|---|---|
| Example 1 | (AA) | 0 | (2-1) | c = 2/X = | (5-1) | | | (2-1) | c = 2/X = |
| | | | | hydroxyl group | | | | | hydroxyl group |
| Example 2 | (AB) | 0 | (2-1) | c = 2/X = | (5-2) | | | (2-1) | c = 2/X = |
| | | | | hydroxyl group | | | | | hydroxyl group |
| Example 3 | (AI) | 0 | (2-2) | a1 = 1/b1 = | (5-1) | | | (2-2) | a1 = 1/b1 = |
| | | | | 2/d = 2 | | | | | 2/d = 2 |
| Example 4 | (AJ) | 0 | (2-2) | a1 = 1/b1 = | (5-2) | | | (2-2) | a1 = 1/b1 = |
| | | | | 2/d = 2 | | | | | 2/d = 2 |
| Example 5 | (AN) | 0 | (2-3) | a2 = 1/b2 = | (5-1) | | | (2-3) | a2 = 1/b2 = |
| | | | | 1/e = 0 | | | | | 1/c = 0 |
| Example 6 | (AO) | 0 | (2-3) | a2 = 1/b2 = | (5-2) | | | (2-3) | a2 = 1/b2 = |
| | | | | 1/e = 0 | | | | | 1/e = 0 |
| Example 7 | (AP) | 0 | (2-3) | a2 = 1/b2 = | (5-1) | | | (2-3) | a2 = 1/b2 = |
| | | | | 2/e = 0 | | | | | 2/e = 0 |
| Example 8 | (AQ) | 0 | (2-3) | a2 = 0/e = 0 | (5-2) | | | (2-3) | a2 = 0/e = 0 |
| Example 9 | (AS) | 0 | (2-4) | a3 = 1/b3 = | (5-2) | | | (2-4) | a3 = 1/b3 = |
| | | | | 2/f = 1 | | | | | 2/f = 1 |
| Example 10 | (AW) | 0 | (2-5) | | (5-2) | | | (2-5) | |
| Example 11 | (BA) | 0 | (2-1) | c = 2/X = | (5-1) | | | (2-2) | a1 = 1/b1 = |
| | | | | hydroxyl group | | | | | 2/d = 2 |
| Example 12 | (BB) | 0 | (2-1) | c = 2/X = | (5-2) | | | (2-2) | a1 = 1/b1 = |
| | | | | hydroxyl group | | | | | 2/d = 2 |
| Example 13 | (BC) | 0 | (2-2) | a1 = 1/b1 = | (5-2) | | | (2-3) | a2 = 1/b2 = |
| | | | | 2/d = 2 | | | | | 2/c = 0 |
| Example 14 | (BD) | 0 | (2-1) | c = 2/X = | (5-2) | | | (3-1) | p = 0/q1 = 1/ |
| | | | | hydroxyl group | | | | | q4 = 2/D = |
| | | | | | | | | | hydroxyl group |
| Example 15 | (BE) | 0 | (2-1) | c = 2/X = | (5-1) | | | (3-2) | r = 1/s1 = |
| | | | | hydroxyl group | | | | | 2/s2 = 1/s3 = 1 |
| Example 16 | (BF) | 0 | (2-2) | a1 = 1/b1 = | (5-2) | | | (3-1) | p = 1/q1 = 2/ |
| | | | | 2/d = 2 | | | | | q2 = 1/q3 = 1/ |
| | | | | | | | | | q4 = 2/D = |
| | | | | | | | | | hydroxyl group |
| Example 17 | (BG) | 0 | (2-2) | a1 = 1/b1 = | (5-2) | | | (3-2) | r = 1/s1 = 2/ |
| | | | | 2/d = 2 | | | | | s2 = 1/s3 = 1 |
| Example 18 | (BH) | 0 | (2-2) | a1 = 1/b1 = | (5-2) | | | 3-2) | r = 1/s1 = 4/ |
| | | | | 2/d = 2 | | | | | s2 = 1/s3 = 1 |
| Example 19 | (BI) | 0 | (2-2) | a1 = 1/b1 = | (5-2) | | | (3-2) | r = 1/s1 = 1/ |
| | | | | 2/d = 2 | | | | | s2 = 2/s3 = 1 |
| Example 20 | (BJ) | 0 | (2-2) | a1 = 1/b1 = | (5-2) | | | (3-2) | r = 1/s1 = 2/ |
| | | | | 2/d = 2 | | | | | s2 = 2/s3 = 1 |
| Example 21 | (BK) | 0 | (2-2) | a1 = 1/b1 = | (5-2) | | | (3-2) | r = 1/s1 = 1/ |
| | | | | 2/d = 2 | | | | | s2 = 1/s3 = 1 |
| Example 22 | (BL) | 0 | (2-2) | a1 = 1/b1 = | (5-2) | | | (3-2) | r = 0/s1 = 1 |
| | | | | 2/d = 2 | | | | | |
| Example 23 | (BM) | 0 | (2-2) | a1 = 1/b1 = | (5-2) | | | (3-4) | g1 = 1/R^{a} = |
| | | | | 2/d = 2 | | | | | H/R^{b} = H |
| Example 24 | (CA) | 1 | (2-1) | c = 2/X = | (5-1) | (4-1) | u1 = 0/ | (2-1) | c = 2/X = |
| | | | | hydroxyl group | | | u2 = 0 | | hydroxyl group |
| Example 25 | (DA) | 0 | (2-6) | f2 = 2 | (5-2) | | | (3-2) | r = 0/s1 = 1 |

### Comparative Example 1

A compound represented by the following formula (ZA) was synthesized by a method described in Patent Document 1.

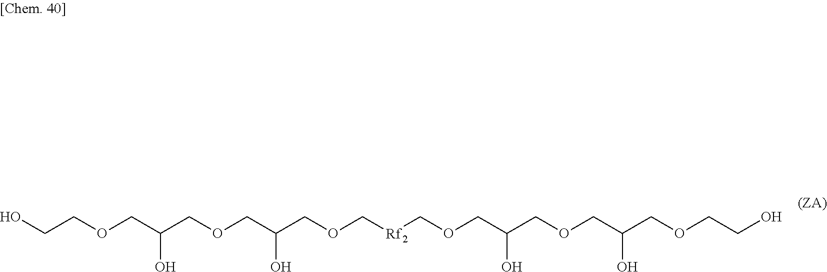

(Rf₂ in the formula (ZA) is the PFPE chain represented by the formula (5-2). In Rf₂, j indicating the average degree of polymerization represents 4.5.)

### Comparative Example 2

A compound represented by the following formula (ZB) was synthesized by a method described in Patent Document 2.

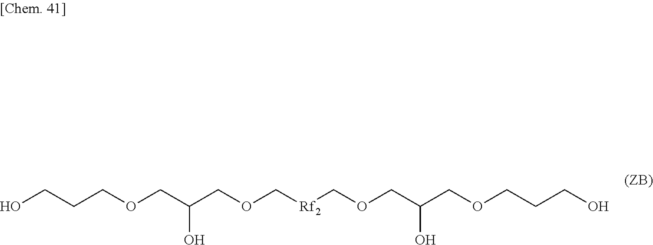

(Rf₂ in the formula (ZB) is the PFPE chain represented by the formula (5-2). In Rf₂, j indicating the average degree of polymerization represents 4.5.)

### Comparative Example 3

A compound represented by the following formula (ZC) was synthesized by a method described in Patent Document 3.

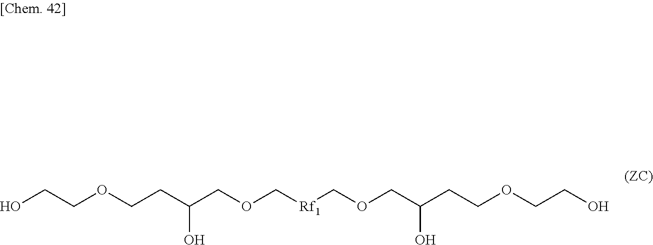

(Rf₁ in the formula (ZC) is the PFPE chain represented by the formula (5-1). In Rf₁, h indicating the average degree of polymerization represents 4.5, and i indicating the average degree of polymerization represents 4.5.)

### Comparative Example 4

A compound represented by the following formula (ZD) was synthesized by the following method.

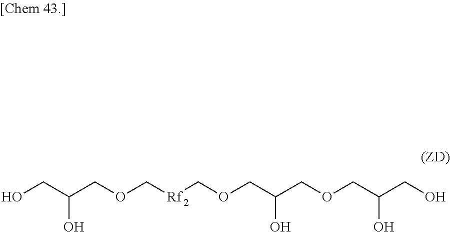

(Rf₂ in the formula (ZD) is the PFPE chain represented by the formula (5-2). In Rf₂, j indicating the average degree of polymerization represents 4.5.)

The same operation as in Example 11 was performed except that the compound represented by the formula (6-14) was used instead of the compound represented by the formula (6-1), the compound represented by the formula (6-15) was used instead of the compound represented by the formula (6-2), and the compound represented by HOCH₂CF₂CF₂O(CF₂CF₂CF₂O)ⱼCF₂CF₂CH₂OH (j indicating the average degree of polymerization in the formula was 4.5) (number-average molecular weight 950, molecular weight distribution 1.1) was used instead of the compound represented by HOCH₂CF₂O(CF₂CF₂O)ₕ(CF₂O)ᵢCF₂CH₂OH, and 2.81 g of a compound (ZD) was obtained.

¹H-NMR and ¹⁹F-NMR measurement of the obtained compound (ZD) was performed, and the structure was identified from the following results.

¹H-NMR (acetone-D₆): δ [ppm]=3.40 to 3.85 (20H), 3.85 to 4.10 (4H)

¹⁹F-NMR (acetone-D₆): δ [ppm]=−84.0 to −83.0 (18F), −86.4 (4F), −124.3 (4F), −130.0 to −129.0 (9F)

### Comparative Example 5

A compound represented by the following formula (ZA) was synthesized by a method described in Patent Document 4.

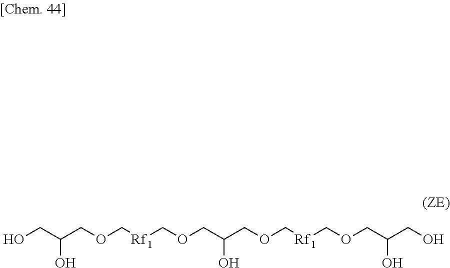

(Two Rf₁'s in the formula (ZE) are the PFPE chains represented by the formula (5-1). In two Rf₁'s, h indicating the average degree of polymerization represents 7.0, and i indicating the average degree of polymerization represents 0.)

The number-average molecular weights (Mn) of the compounds of Examples 1 to 24 and Comparative Examples 1 to 5 obtained as described above were measured by the above-described method. The results are shown in Table 2.

Next, solutions for forming a lubricating layer were prepared using the compounds obtained in Examples 1 to 24 and Comparative Examples 1 to 5 by a method to be described below. In addition, lubricating layers for magnetic recording media were formed by a method to be described below using the obtained solutions for forming a lubricating layer, and magnetic recording media of Examples 1 to 24 and Comparative Examples 1 to 5 were obtained.

### Solutions for Forming Lubricating Layer

The compounds obtained in Examples 1 to 24 and Comparative Examples 1 to 5 were each dissolved in VERTREL (registered trademark) XF (trade name, manufactured by Du Pont-Mitsui Fluorochemicals Company, Ltd.), which is a fluorine-based solvent, and were diluted with VERTREL XF so that the film thickness reached 8.0 Å to 8.5 Å when the compound was applied onto a protective layer, thereby producing the solutions for forming a lubricating layer.

### Magnetic Recording Media

Magnetic recording media each having an adhesive layer, a soft magnetic layer, a first base layer, a second base layer, a magnetic layer, and a protective layer provided in order on a substrate having a diameter of 65 mm were prepared. The protective layers were made of carbon.

The solutions for forming a lubricating layer of Examples 1 to 24 and Comparative Examples 1 to 5 were each applied onto the protective layer of the magnetic recording medium in which the individual layers up to the protective layer had been formed by a dipping method. The dipping method was performed under conditions of an immersion rate of 10 mm/sec, an immersion time of 30 sec, and a lifting rate of 1.2 mm/sec.

After that, the magnetic recording medium onto which the solution for forming a lubricating layer had been applied was put into a thermostatic chamber, and a thermal treatment for improving the adhesion between the protective layer and the lubricating layer by removing the solvent in the solution for forming a lubricating layer was performed at 120° C. for 10 minutes, thereby forming a lubricating layer on the protective layer and obtaining a magnetic recording medium.

### Film Thickness Measurement

The film thicknesses of the lubricating layers in the magnetic recording media of Examples 1 to 24 and Comparative Examples 1 to 5 obtained as described above were measured using Fourier transform infrared spectroscopy (FT-IR, trade name: Nicolet iS50, manufactured by Thermo Fisher Scientific Inc.). The results are shown in Table 2.

Next, a chemical resistance test and a spinoff characteristic test, which will be described below, were performed on the magnetic recording media of Examples 1 to 25 and Comparative Examples 1 to 5. The results are shown in Table 2.

### Chemical Resistance Test

Contamination of a magnetic recording medium by an environmental substance that generated a contamination substance under a high-temperature environment was examined by a method to be described below. Si ions were used as the environmental substance, and the amount of Si adsorbed was measured as the amount of the contamination substance that was generated by the environmental substance and contaminated the magnetic recording medium.

Specifically, a magnetic recording medium, which was an evaluation subject, was held for 240 hours under a high-temperature environment with a temperature of 85° C. and a humidity of 0% in the presence of siloxane-based Si rubber. Next, the amount of Si adsorbed present on the surface of the magnetic recording medium was analyzed and measured by secondary-ion mass spectrometry (SIMS), and the degree of contamination caused by the Si ions was evaluated as the amount of Si adsorbed. The evaluation of the amount of Si adsorbed was evaluated based on the following evaluation standards using a numerical value obtained when the result of the amount of Si adsorbed in Comparative Example 4 was regarded as 1.00. The results are shown in Table 2.

### Evaluation Standards of Chemical Substance Resistance

A+: The amount of Si adsorbed is less than 0.60
A: The amount of Si adsorbed is 0.60 or more and less than 0.70
B: The amount of Si adsorbed is 0.70 or more and less than 0.90
C: The amount of Si adsorbed is 0.90 or more and less than 1.10
D: The amount of Si adsorbed is 1.10 or more

### Spinoff Characteristic Test

A magnetic recording medium was mounted on a spin stand and rotated at a rotation speed of 10000 rpm for 72 hours in an environment of 80° C. Before and after this operation, the film thicknesses of the lubricating layer at a position of a radius of 20 mm from the center of the magnetic recording medium were measured using FT-IR, and the film thickness decrease rate of the lubricating layer before and after the test was calculated. The spinoff characteristics were evaluated using the calculated film thickness decrease rate according to the evaluation standards shown below.

### Evaluation Standards of Spinoff Characteristics

A+: The film thickness decrease rate is less than 2%
A: The film thickness decrease rate is 2% or more and less than 3%
B: The film thickness decrease rate is 3% or more and less than 5%
C: The film thickness decrease rate is 5% or more and less than 10%
D: The film thickness decrease rate is 10% or more

### Comprehensive Evaluation

Comprehensive evaluation was performed based on the following standards from the results of the re and the spinoff characteristic test.

### Comprehensive Evaluation Standards

A: The evaluation of the chemical resistance test and the evaluation of the spinoff characteristic test are both A+ or A.
B: One of the evaluation of the chemical resistance test and the evaluation of the spinoff characteristic test is B, and the other is A+, A, or B.
C: One of the evaluation of the chemical resistance test and the evaluation of the spinoff characteristic test is C, and the other is A+, A, B, or C.
D: At least one of the evaluation of the chemical resistance test and the evaluation of the spinoff characteristic test is D.

TABLE 2

| | | | Film | Chemical | Spinoff | |
|---|---|---|---|---|---|---|
| | | Molecular | thickness | resistance | characteristic | Comprehensive |
| | Compound | weight | (Å) | test | test | evaluation |
| Example 1 | (AA) | 1047 | 8.5 | A | A | A |
| Example 2 | (AB) | 1214 | 8.2 | A | A | A |
| Example 3 | (AI) | 1261 | 8.2 | A+ | A | A |
| Example 4 | (AJ) | 1439 | 8.3 | A+ | A | A |
| Example 5 | (AN) | 1165 | 8.1 | A | A | A |
| Example 6 | (AO) | 1340 | 8.4 | A | A | A |
| Example 7 | (AP) | 1234 | 8.0 | A | A | A |
| Example 8 | (AQ) | 1188 | 8.1 | A+ | B | B |
| Example 9 | (AS) | 1581 | 8.4 | A+ | A | A |
| Example 10 | (AW) | 1164 | 8.3 | A | A | A |
| Example 11 | (BA) | 1153 | 8.5 | A | A | A |
| Example 12 | (BB) | 1342 | 8.2 | A | A | A |
| Example 13 | (BC) | 1192 | 8.0 | A | A | A |
| Example 14 | (BD) | 1195 | 8.2 | A | A | A |
| Example 15 | (BE) | 1251 | 8.0 | A | A | A |
| Example 16 | (BF) | 1384 | 8.0 | A | A | A |
| Example 17 | (BG) | 1347 | 8.1 | A | A | A |
| Example 18 | (BH) | 1375 | 8.0 | A | A | A |
| Example 19 | (BI) | 1351 | 8.1 | A | A | A |
| Example 20 | (BJ) | 1363 | 8.1 | A | A | A |
| Example 21 | (BK) | 1332 | 8.2 | A | A | A |
| Example 22 | (BL) | 1260 | 8.2 | A | A | A |
| Example 23 | (BM) | 1392 | 8.5 | A | B | B |
| Example 24 | (CA) | 1846 | 8.1 | A | A+ | A |
| Example 25 | (DA) | 1151 | 8.2 | A | A | A |
| Comparative | (ZA) | 1380 | 8.0 | D | B | D |
| Example 1 | | | | | | |
| Comparative | (ZB) | 1267 | 8.2 | C | D | D |
| Example 2 | | | | | | |
| Comparative | (ZC) | 1274 | 8.3 | C | D | D |
| Example 3 | | | | | | |
| Comparative | (ZD) | 1175 | 8.3 | C | C | C |
| Example 4 | | | | | | |
| Comparative | (ZE) | 2206 | 8.2 | C | C | C |
| Example 5 | | | | | | |

As shown in Table 2, for all of the magnetic recording media of Examples 1 to 25 for which the fluorine-containing ether compound satisfying the formula (1) in which at least one of R¹ and R⁴ disposed at the terminals of a skeleton including a perfluoropolyether chain was the terminal group represented by the formula (2) was used, the evaluations of the chemical resistance test and the spinoff characteristic test were any of “A+,” “A,” or “B,” and the comprehensive evaluations were “A” or “B.” From these facts, it was possible to confirm that the lubricating layers in the magnetic recording media of Examples 1 to 25 had favorable chemical substance resistance and were capable of curbing spinoff.

In contrast, as shown in Table 2, for all of the magnetic recording media of Comparative Examples 1 to 5 for which the compounds (ZA) to (ZE) including no erythritol structures were used, the evaluations of the chemical resistance test and the spinoff characteristic test were any of “B,” “C,” or “D,” and the comprehensive evaluations were “C” or “D.”

In more detail, all of the hydroxyl groups that were included in the compounds (ZA) to (ZC) used in Comparative Examples 1 to 3 significantly contribute to an increase in the polarity of the entire molecule and increase the surface free energy of the fluorine-containing ether compounds. In addition, the hydroxyl groups that were included in the compounds (ZA) to (ZC) are not structures in which the orientations of adjacent hydroxyl groups are not in reversed conformation on the protective layer, are all disposed to easily interact with active points on the protective layer, are likely to get involved in the interaction with the protective layer, but are less likely to get involved in intermolecular interactions.

In addition, the compound (ZA) includes six hydroxyl groups, but no hydroxyl groups of an erythritol structure are included therein. Therefore, it is considered that the surface free energy of the fluorine-containing ether compound becomes too high due to a large number of the hydroxyl groups and the lubricating layer for which the fluorine-containing ether compound is used is likely to attract chemical substances from the environment and evaluated as “D” in the result of the chemical resistance test.

In addition, the compounds (ZB) and (ZC) include four hydroxyl groups, but no hydroxyl groups of an erythritol structure are included therein. Therefore, it is considered that the hydroxyl groups that are included in the compounds (ZB) and (ZC) are all likely to get involved in the interaction with the protective layer and evaluated as “D” in the results of the chemical resistance test due to the lack of hydroxyl groups that get involved in intermolecular interactions.

In addition, in the compound (ZD) used in Comparative Example 4 and the compound (ZE) used in Comparative Example 5, the 1,2-diol structures (—O—CH₂—CH(OH)—CH₂OH) were disposed at both terminals. In the fluorine-containing ether compounds having the 1,2-diol structures disposed at the terminals, two hydroxyl groups in the 1,2-diol structure are each sufficiently mobile compared with hydroxyl groups in the erythritol structure, and the surface free energy is thus less likely to decrease. From this fact, it is considered that the lubricating layers for which the fluorine-containing ether compound is used are likely to attract chemical substance from the environment and each evaluated as “C” in the results of the chemical resistance test. In addition, since the terminal groups in the compounds (ZD) and (ZE) are highly flexible, the hydroxyl groups that are included in the compounds are likely to form intramolecular interactions. As a result, it is considered that the results of the spinoff characteristic test were each “C” due to the lack of hydroxyl groups that get involved in intermolecular interactions.

### INDUSTRIAL APPLICABILITY

The use of a lubricant for a magnetic recording medium containing the fluorine-containing ether compound of the present invention makes it possible to form a lubricating layer having favorable chemical substance resistance and being capable of curbing spinoff even when the thickness is thin.

### REFERENCE SIGNS LIST

10 Magnetic recording medium
11 Substrate
12 Adhesive layer
13 Soft magnetic layer
14 First base layer
15 Second base layer
16 Magnetic layer
17 Protective layer
18 Lubricating layer

1. A fluorine-containing ether compound that is represented by a following formula (1): 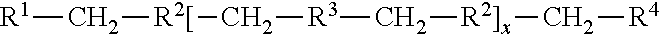
(in the formula (1), x represents an integer of 0 to 2; R² is a perfluoropolyether chain; in a case where x is 1 or 2, (x+1) R²'s may be partially or fully identical to each other or may be different from each other; R³ is a divalent linking group having 1 to 4 polar groups; in a case where x is 2, two R³'s may be identical to each other or may be different from each other; R¹ and R⁴ are each independently a terminal group having 1 to 4 polar groups and having 1 to 50 carbon atoms; and at least one of R¹ and R⁴ is a terminal group represented by a following formula (2)) 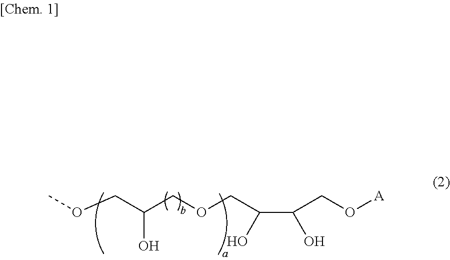
(in the formula (2), a represents an integer of 0 to 2, and b represents an integer of 1 to 6; A is an organic group or a hydrogen atom, the organic group having 1 to 30 carbon atoms and optionally including at least one of a polar group, a halogeno group, and an ether oxygen atom; here, the total number of the polar groups included in the formula (2) is 2 to 4; and in a case where a is 2, two b's may be identical to each other or may be different from each other).

2. The fluorine-containing ether compound according to claim 1, wherein the terminal group represented by the formula (2) is any of following formulae (2-1) to (2-6): 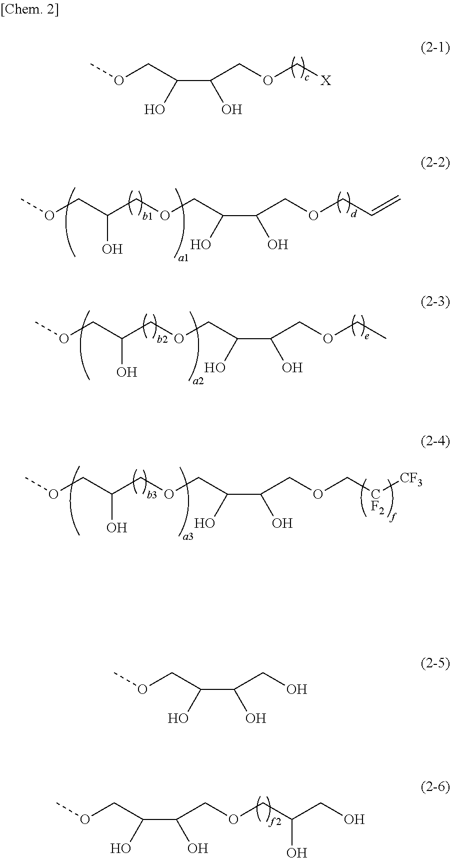
(in the formula (2-1), c represents an integer of 1 to 6; and X represents any of a hydroxyl group, an acetamide group, a carboxamide group, and a cyano group),
(in the formula (2-2), a1 represents 0 or 1; bi represents an integer of 1 to 6; and d represents an integer of 1 to 4),
(in the formula (2-3), a2 represents 0 or 1; b2 represents an integer of 1 to 6; and e represents an integer of 0 to 7),
(in the formula (2-4), a3 represents 0 or 1; b3 represents an integer of 1 to 6; and f represents an integer of 0 to 6), and
(in the formula (2-6), f2 represents an integer of 1 to 6).

3. The fluorine-containing ether compound according to claim 1, wherein R¹ and R⁴ in the formula (1) are each independently the terminal group represented by the formula (2).

4. The fluorine-containing ether compound according to claim 3, wherein R¹ and R⁴ in the formula (1) are identical to each other.

5. The fluorine-containing ether compound according to claim 1, wherein only one of R¹ and R⁴ in the formula (1) is the terminal group represented by the formula (2), and other is a terminal group represented by a formula (3), 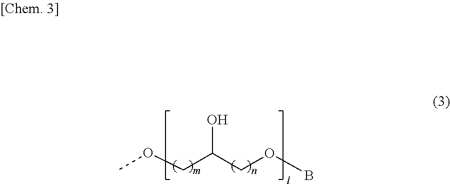
(in the formula (3), 1 represents an integer of 1 to 3; 1 m's each independently represent an integer of 1 to 6; 1 n's each independently represent an integer of 1 to 6; in one repeating unit, at least one of m and n is 1; and B represents an alkyl group, an organic group, or a hydrogen atom, the alkyl group optionally having only one polar group, the organic group including a carbon-carbon unsaturated bond and optionally having only one polar group).

6. The fluorine-containing ether compound according to claim 1, wherein only one of R¹ and R⁴ in the formula (1) is the terminal group represented by the formula (2), and other is a terminal group represented by any of following formulae (3-1) to (3-3): 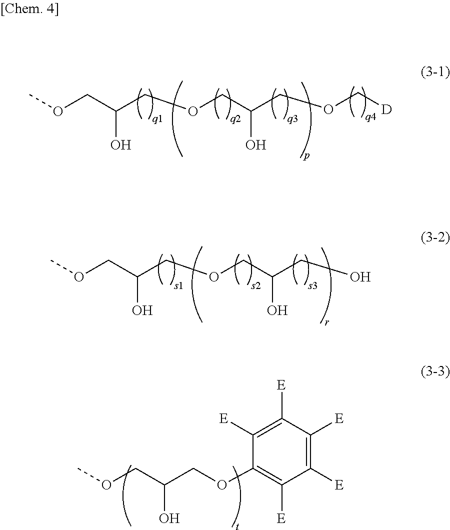
(in the formula (3-1), p represents 0 or 1; q1, q2, q3, and q4 each independently represent an integer of 1 to 6; in a case where p is 0, a total value of q1 and q4 is 2 to 10; in a case where p is 1, a total value of q1, q2, q3, and q4 is 4 to 10; in a case where p is 1, at least one of q2 and q3 is 1; D represents a polar group, a vinyl group, an ethynyl group, or an aryl group, the aryl group optionally having a substituent; here, in a case where D is the aryl group optionally having a substituent, a number of polar groups included in D is 0 or 1),
(in the formula (3-2), r represents 0 or 1; s1, s2, and s3 each independently represent an integer of 1 to 6; in a case where r is 1, a total value of s1, s2, and s3 is 3 to 8; and in a case where r is 1, at least one of s2 and s3 is 1), and
(in the formula (3-3), t represents 1 or 2; five E's each independently represent a polar group, an alkoxy group, a halogeno group, or a hydrogen atom, the alkoxy group having 1 to 8 carbon atoms; and in a case where a polar group is included in the five E's, a total number of the polar groups in the five E's is one).

7. The fluorine-containing ether compound according to claim 1, wherein only one of R¹ and R⁴ in the formula (1) is the terminal group represented by the formula (2), and other is a terminal group represented by following formulae (3-4) or (3-5): 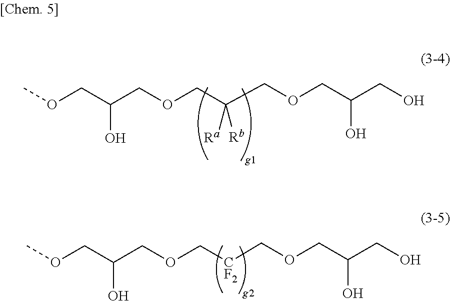
(in the formula (3-4), g1 represents an integer of 1 to 6; and g1 R^{a}'s and g1 R^{b}'s are each independently a hydrogen atom or a methyl group), and
(in the formula (3-5), g2 represents an integer of 1 to 6).

8. The fluorine-containing ether compound according to claim 1, wherein x in the formula (1) is 1 or 2, and x R³'s are each independently a divalent linking group having one to three hydroxyl groups, having oxygen atoms at both end portions bonding to adjacent methylene groups, and having 3 to 50 carbon atoms.

9. The fluorine-containing ether compound according to claim 1, wherein x in the formula (1) is 1 or 2, and x R³'s are each independently any one selected from linking groups represented by following formulae (4-1) to (4-6): 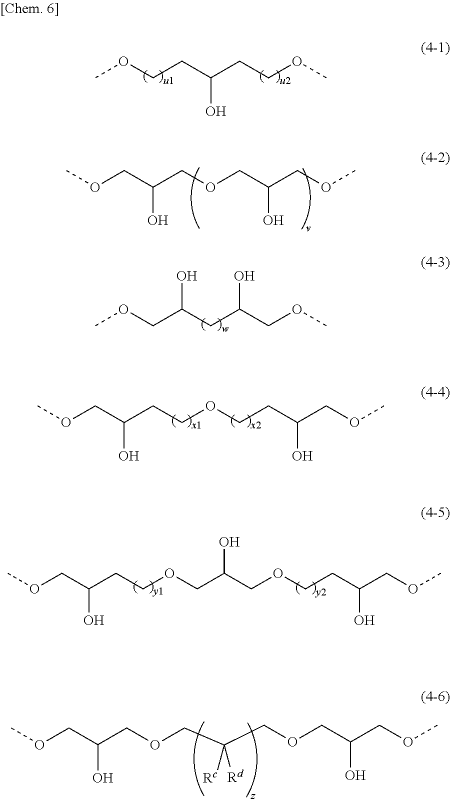
(in the formula (4-1), u1 represents an integer of 0 to 6; u2 represents an integer of 0 to 6; here, at least one of u1 and u2 is 0; and an oxygen atom at a left terminal of the formula (4-1) bonds to a methylene group on an R¹ side in the formula (1) and an oxygen atom at a right terminal bonds to a methylene group on an R⁴ side in the formula (1)),
(in the formula (4-2), v represents an integer of 1 or 2; and an oxygen atom at a left terminal of the formula (4-2) bonds to the methylene group on the R¹ side in the formula (1) and an oxygen atom at a right terminal bonds to the methylene group on the R⁴ side in the formula (1)),
(in the formula (4-3), w represents an integer of 0 to 6; an oxygen atom at a left terminal of the formula (4-3) bonds to the methylene group on the R¹ side in the formula (1) and an oxygen atom at a right terminal bonds to the methylene group on the R⁴ side in the formula (1)),
(in the formula (4-4), x1 represents an integer of 0 to 5; x2 represents an integer of 0 to 5; here, at least one of x1 and x2 is an integer of 1 to 5; and an oxygen atom at a left terminal of the formula (4-4) bonds to the methylene group on the R¹ side in the formula (1); and an oxygen atom at a right terminal bonds to the methylene group on the R⁴ side in the formula (1)),
(in the formula (4-5), y1 represents an integer of 1 to 5; y2 represents an integer of 1 to 5; and an oxygen atom at a left terminal of the formula (4-5) bonds to the methylene group on the R¹ side in the formula (1) and an oxygen atom at a right terminal bonds to the methylene group on the R⁴ side in the formula (1)), and
(in the formula (4-6), z represents an integer of 1 to 6; z R^{c}'s and z R^{d}'s are each independently a hydrogen atom, a fluorine atom, or a methyl group; and an oxygen atom at a left terminal of the formula (4-6) bonds to the methylene group on the R¹ side in the formula (1) and an oxygen atom at a right terminal bonds to the methylene group on the R⁴ side in the formula (1)).

10. The fluorine-containing ether compound according to claim 1, wherein (x+1) R²'s in the formula (1) are each independently a perfluoropolyether chain represented by a following formula (5): 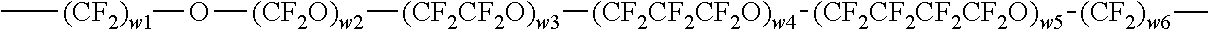
(in the formula (5), w2, w3, w4, and w5 indicate average degrees of polymerization and each independently represent 0 to 20; here, w2, w3, w4, and w5 are not all 0 at the same time; w1 and w6 are average values representing the numbers of CF₂'s and each independently represent 1 to 3; and an array order of (CF₂O), (CF₂CF₂O), (CF₂CF₂CF₂O), and (CF₂CF₂CF₂CF₂O), which are repeating units in the formula (5), is not particularly limited).

11. The fluorine-containing ether compound according to claim 1, wherein (x+1) R²'s in the formula (1) are each independently any one selected from perfluoropolyether chains represented by following formulae (5-1) to (5-4): 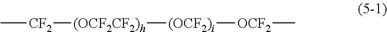
(in the formula (5-1), h and i indicate average degrees of polymerization; h represents 1 to 20; and i represents 0 to 20), 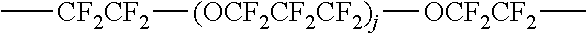
(in the formula (5-2), j indicates an average degree of polymerization and represents 1 to 15), 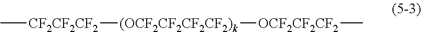
(in the formula (5-3), k indicates an average degree of polymerization and represents 1 to 10), 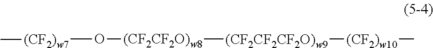
(in the formula (5-4), w8 and w9 indicate average degrees of polymerization and each independently represent 1 to 20; and w7 and w10 are average values representing a numbers of CF₂'s and each independently represent 1 or 2).

12. The fluorine-containing ether compound according to claim 1, wherein the fluorine-containing ether compound represented by the formula (1) is any one of following formulae (AA), (AB), (AI), (AJ), (AN) to (AQ), (AS), (AW), (BA) to (BM), (CA), and (DA),
Rf₁ is the perfluoropolyether chain represented by a following formula (5-1), and
Rf₂ is the perfluoropolyether chain represented by a following formula (5-2): 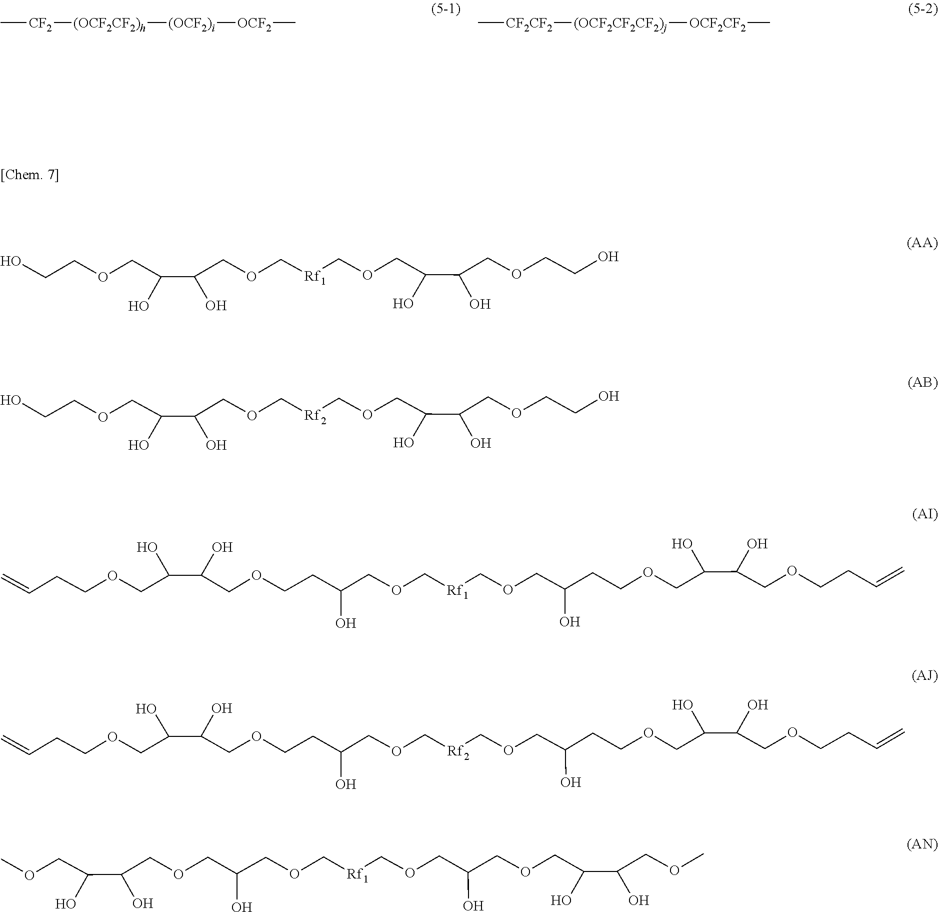
(in Rf₁ in the formula (AA), h and i indicate average degrees of polymerization; h represents 1 to 20; and i represents 0 to 20),
(in Rf₂ in the formula (AB), j indicates an average degree of polymerization and represents 1 to 15),
(in Rf₁ in the formula (AI), h and i indicate average degrees of polymerization; h represents 1 to 20; and i represents 0 to 20),
(in Rf₂ in the formula (AJ), j indicates an average degree of polymerization and represents 1 to 15), and
(in Rf₁ in the formula (AN), h and i indicate average degrees of polymerization; h represents 1 to 20; and i represents 0 to 20) 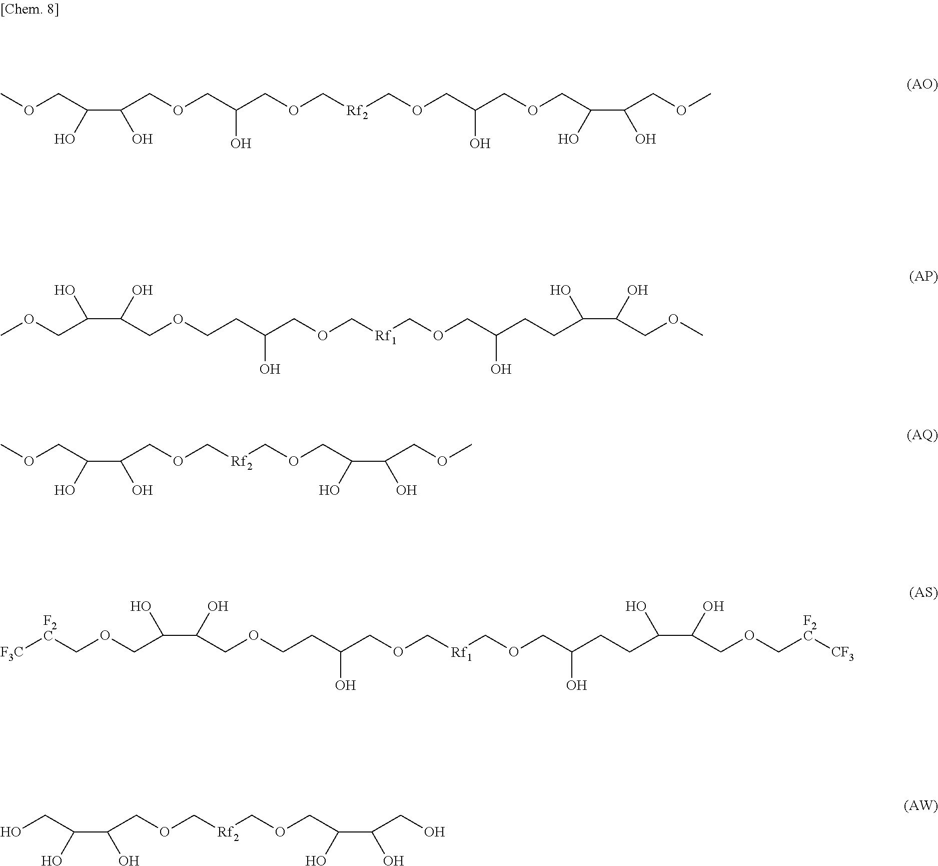
(in Rf₂ in the formula (AO), j indicates an average degree of polymerization and represents i to 15),
(in Rf₁ in the formula (AP), h and i indicate average degrees of polymerization; h represents 1 to 20; and i represents 0 to 20),
(in Rf₂ in the formula (AQ), j indicates an average degree of polymerization and represents 1 to 15),
(in Rf₂ in the formula (AS), j indicates an average degree of polymerization and represents 1 to 15), and
(in Rf₂ in the formula (AW), j indicates an average degree of polymerization and represents 1 to 15) 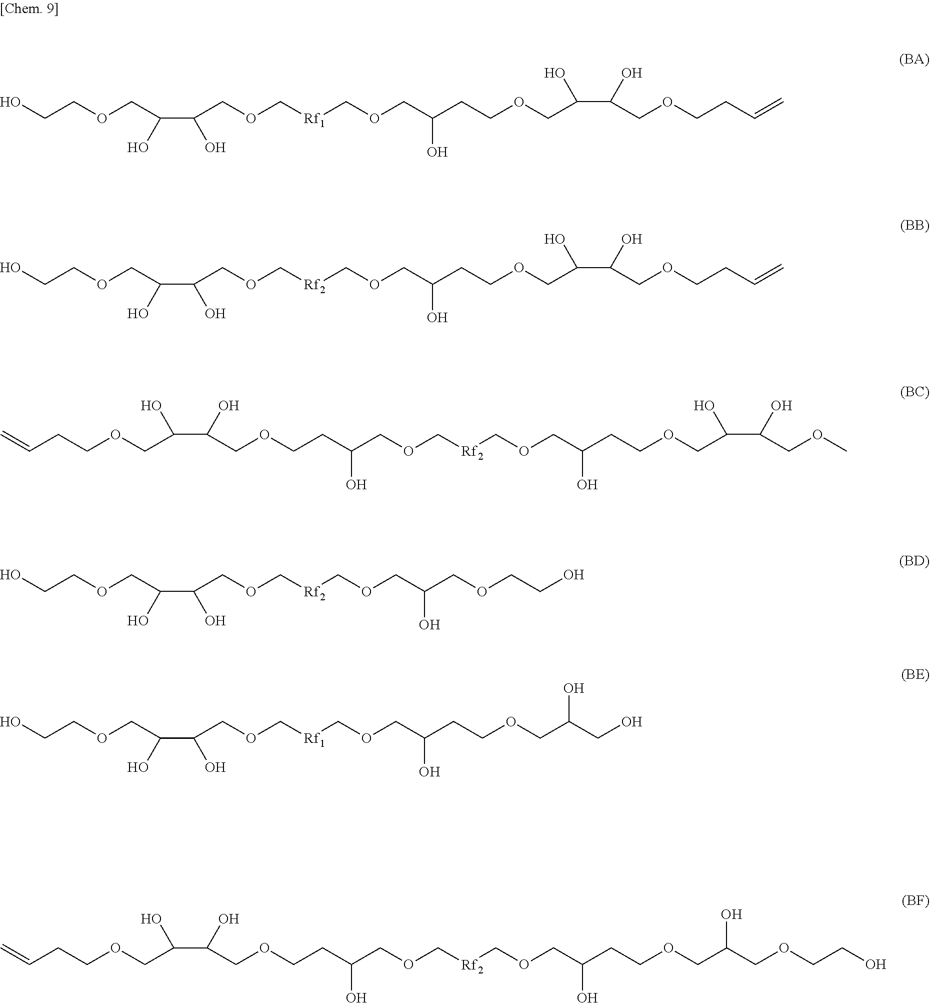
(in Rf₁ in the formula (BA), h and i indicate average degrees of polymerization; h represents 1 to 20; and i represents 0 to 20),
(in Rf₂ in the formula (BB), j indicates an average degree of polymerization and represents 1 to 15),
(in Rf₂ in the formula (BC), j indicates an average degree of polymerization and represents 1 to 15),
(in Rf₂ in the formula (BD), j indicates an average degree of polymerization and represents 1 to 15),
(in Rf₁ in the formula (BE), h and i indicate average degrees of polymerization; h represents 1 to 20; and i represents 0 to 20), and
(in Rf₂ in the formula (BF), j indicates an average degree of polymerization and represents 1 to 15) 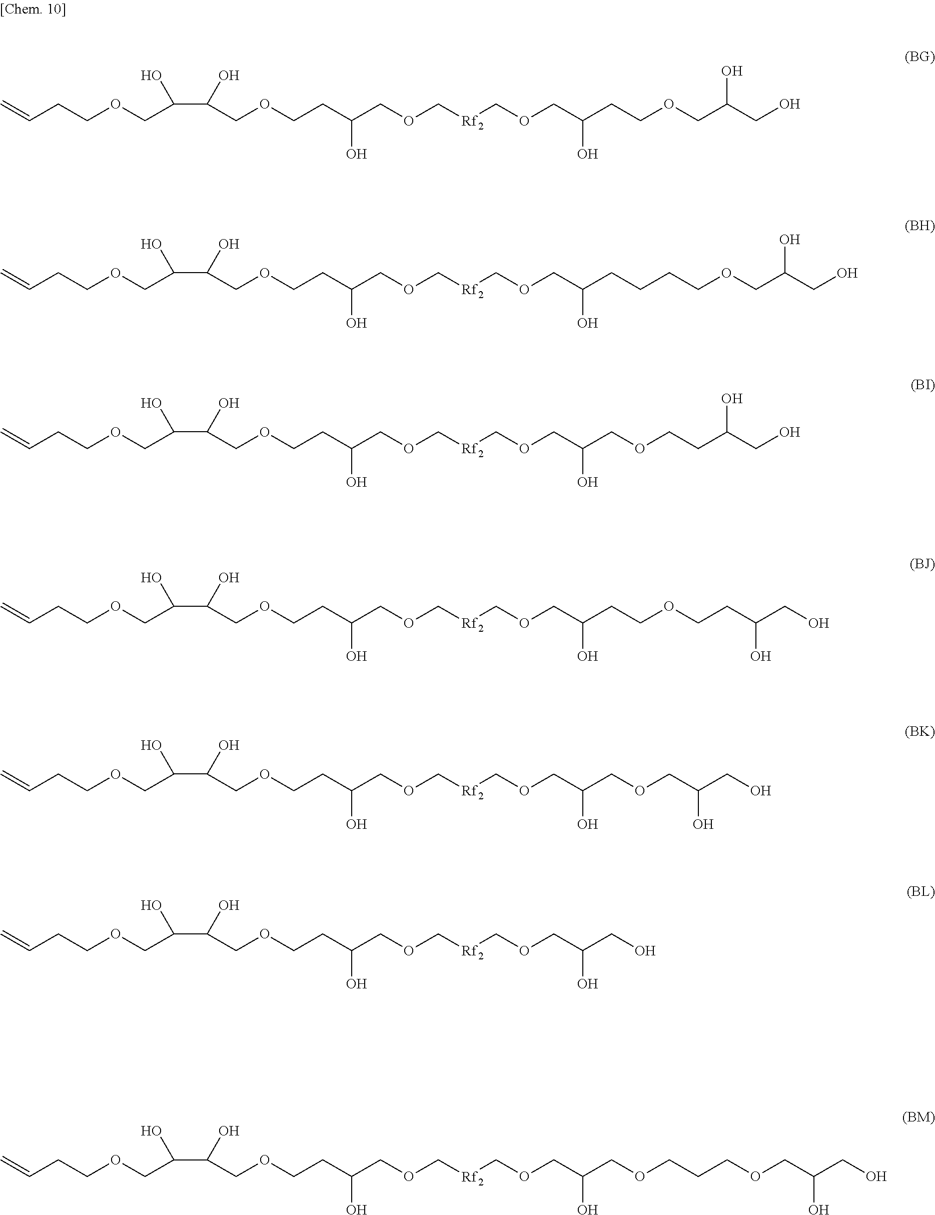
(in Rf₂ in the formula (BG), j indicates an average degree of polymerization and represents 1 to 15),
(in Rf₂ in the formula (BH), j indicates an average degree of polymerization and represents 1 to 15),
(in Rf₂ in the formula (BI), j indicates an average degree of polymerization and represents 1 to 15),
(in Rf₂ in the formula (BJ), j indicates an average degree of polymerization and represents 1 to 15),
(in Rf₂ in the formula (BK), j indicates an average degree of polymerization and represents 1 to 15),
(in Rf₂ in the formula (BL), j indicates an average degree of polymerization and represents 1 to 15), and
(in Rf₂ in the formula (BM), j indicates an average degree of polymerization and represents 1 to 15) 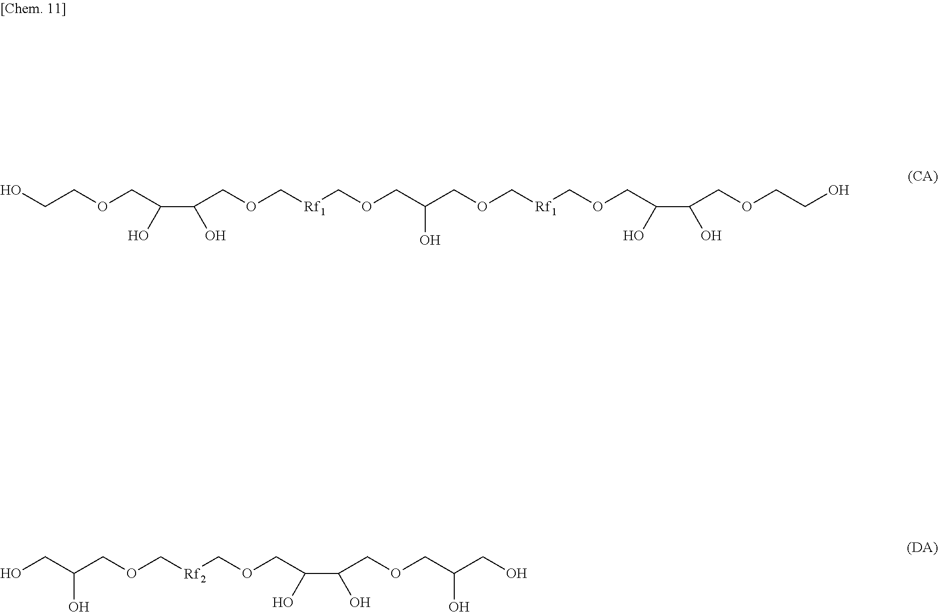
(in two Rf₁'s in the formula (CA), h and i indicate average degrees of polymerization; h represents 1 to 20, i represents 0 to 20; and h's and i's in the two Rf₁'s may be identical to each other or may be different from each other) and
(in Rf₂ in the formula (DA), j indicates an average degree of polymerization and represents 1 to 15).

13. The fluorine-containing ether compound according to claim 1, wherein a number-average molecular weight is within a range of 500 to 10000.

14. A lubricant for a magnetic recording medium containing the fluorine-containing ether compound according to claim 1.

15. A magnetic recording medium having at least a magnetic layer, a protective layer, and a lubricating layer provided in order on a substrate,
wherein the lubricating layer contains the fluorine-containing ether compound according to claim 1.

16. The magnetic recording medium according to claim 15, wherein the lubricating layer has an average film thickness of 0.5 nm to 2.0 nm.
